(19) United States
(12) Patent Application Publication
(10) Pub. No.: US 2024/0090320 A1
(43) Pub. Date: Mar. 14, 2024

(54) LIGHT EMITTING ELEMENT AND AMINE COMPOUND FOR THE SAME

(71) Applicant: Samsung Display Co., LTD., Yongin-si (KR)

(72) Inventors: MINJI KIM, Yongin-si (KR); HANKYU PAK, Yongin-si (KR); HYUNBIN PARK, Yongin-si (KR); BYEONGWOOK YOO, Yongin-si (KR); JEONG MIN LEE, Yongin-si (KR); SOHEE JO, Yongin-si (KR)

(21) Appl. No.: 18/319,199

(22) Filed: May 17, 2023

(30) Foreign Application Priority Data
Aug. 10, 2022 (KR) 10-2022-0100191

(51) Int. Cl. H10K 85/60; H10K 50/11; H10K 50/15

(52) U.S. Cl. CPC H10K 85/633; H10K 50/11; H10K 50/15

(57) ABSTRACT

The present disclosure relates to a light emitting element and an amine compound for the light emitting element, and the light emitting element of an embodiment includes a first electrode, a second electrode on the first electrode, and at least one functional layer between the first electrode and the second electrode, wherein an amine compound represented by Formula 1 is included in the at least one functional layer, thereby improving emission efficiency and element life of the light emitting element.

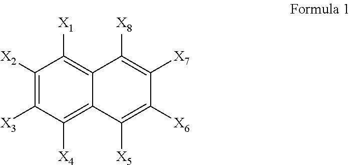

### CROSS-REFERENCE TO RELATED APPLICATION

This application claims priority to and the benefit of Korean Patent Application No. 10-2022-0100191, filed on Aug. 10, 2022, the entire contents of which are hereby incorporated by reference.

### BACKGROUND

### 1. Field

Embodiments of the present disclosure herein relate to a light emitting element and an amine compound for a light emitting element, and for example, to a light emitting element including the amine compound in a functional layer.

### 2. Description of Related Art

Recently, the development of an organic electroluminescence display device as an image display device is being actively conducted. The organic electroluminescence display device is a so-called display device including a self-luminescent-type light emitting element in which holes and electrons injected from a first electrode and a second electrode recombine in an emission layer so that a light emitting material in the emission layer emits light to achieve a display.

In the application of a light emitting element to a display device, the increase of emission efficiency and lifetime is desirable, and development for materials for a light emitting element, stably achieving the requirements is consistently being investigated.

For example, in order to accomplish a light emitting element having high efficiency and long lifetime, development of materials for a hole transport region having excellent hole transport properties and stability is being conducted.

### SUMMARY

Embodiments of the present disclosure provide a light emitting element showing high efficiency and long-life characteristics and an amine compound which is included in the light emitting element.

A light emitting element according to an embodiment of the present disclosure includes a first electrode, a second electrode on the first electrode, and at least one functional layer between the first electrode and the second electrode and including an amine compound represented by Formula 1.

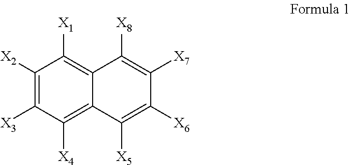

In Formula 1, X₁ is a hydrogen atom, a deuterium atom, a halogen atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or represented by Formula 2, X₂ is a substituted or unsubstituted alkyl group of 1 to 4 carbon atoms, or represented by Formula 2, X₃ to X₈ are each independently a hydrogen atom, a deuterium atom, a halogen atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, any one selected from among X₁ and X₂ is represented by Formula 2, if X₁ is represented by Formula 2, X₂ is a substituted or unsubstituted alkyl group of 1 to 4 carbon atoms, and if X₂ is represented by Formula 2, any one selected from among X₁ and X₃ is a substituted or unsubstituted alkyl group of 1 to 4 carbon atoms.

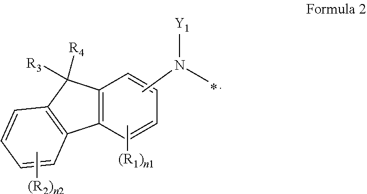

In Formula 2, R₁ to R₄ are each independently a hydrogen atom, a deuterium atom, a halogen atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, Y₁ is a substituted or unsubstituted alkyl group of 1 to 20 carbon atoms, a substituted or unsubstituted alkenyl group of 2 to 20 carbon atoms, a substituted or unsubstituted alkynyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, n1 is an integer of 0 to 3, n2 is an integer of 0 to 4, and “-*” is a position connected with Formula 1.

In the light emitting element according to an embodiment of the present disclosure, the at least one functional layer may include an emission layer, a hole transport region between the first electrode and the emission layer, and an electron transport region between the emission layer and the second electrode, and the hole transport region may include the amine compound represented by Formula 1.

In an embodiment, the hole transport region may include a hole injection layer on the first electrode, and a hole transport layer on the hole injection layer, and the hole transport layer may include the amine compound represented by Formula 1. The amine compound represented by Formula 1 may be a monoamine compound.

In Formula 2, Y₁ may be a substituted or unsubstituted phenyl group, a substituted or unsubstituted naphthyl group, a substituted or unsubstituted fluorenyl group, or a substituted or unsubstituted carbazole group.

In an embodiment, the amine compound represented by Formula 1 may be represented by any one selected from among Formula 1-1 to Formula 1-3.

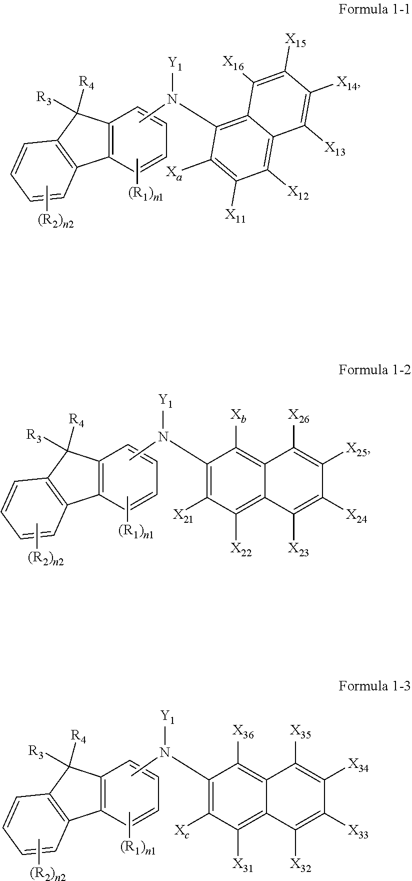

In Formula 1-1 to Formula 1-3, Xₐ, X_{b} and X_{c} are each independently a substituted or unsubstituted alkyl group of 1 to 4 carbon atoms, X₁₁ to X₃₆ are each independently a hydrogen atom, a deuterium atom, a halogen atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and R₁ to R₄, n1, n2 and Y₁ may be the same as defined with respect to Formula 2. In Formula 1-1 to Formula 1-3, Xₐ to X_{c} may be each independently a substituted or unsubstituted methyl group, and X₁₁ to X₃₆ may be each independently a hydrogen atom or a deuterium atom.

In an embodiment, the substituent represented by Formula 1 may be represented by Formula 2-1.

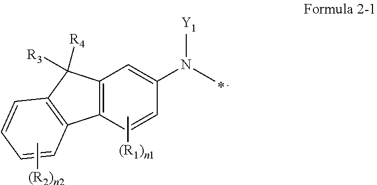

In Formula 2-1, R₁ to R₄, n1, n2 and Y₁ are the same as defined with respect to Formula 2.

In an embodiment, the substituent represented by Formula 2 may be represented by Formula 2-2.

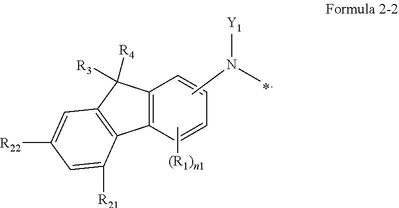

In Formula 2-2, R₂₁ and R₂₂ are each independently a hydrogen atom, a substituted or unsubstituted alkyl group of 1 to 20 carbon atoms, or a substituted or unsubstituted aryl group of 6 to 30 ring-forming carbon atoms, and R₁, R₃, R₄, n1 and Y₁ may be the same as defined with respect to Formula 2.

In an embodiment, the substituent represented by Formula 2 may be represented by Formula 2-3.

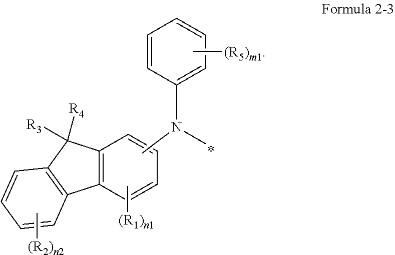

In Formula 2-3, R₅ is a hydrogen atom, a deuterium atom, a halogen atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring, m1 is an integer of 0 to 5, and R₁ to R₄, n1 and n2 may be the same as defined with respect to Formula 2.

An amine compound according to an embodiment of the present disclosure may be represented by Formula 1.

### BRIEF DESCRIPTION OF THE DRAWINGS

The accompanying drawings are included to provide a further understanding of the present disclosure and are incorporated in and constitute a part of this specification. The drawings illustrate embodiments of the present disclosure and, together with the description, serve to explain principles of the present disclosure. In the drawings:

FIG. 1 is a plan view showing a display device according to an embodiment;

FIG. 2 is a cross-sectional view of a display device according to an embodiment;

FIG. 3 is a cross-sectional view schematically showing a light emitting element according to an embodiment;

FIG. 4 is a cross-sectional view schematically showing a light emitting element according to an embodiment;

FIG. 5 is a cross-sectional view schematically showing a light emitting element according to an embodiment;

FIG. 6 is a cross-sectional view schematically showing a light emitting element according to an embodiment;

FIG. 7 is a cross-sectional view of a display device according to an embodiment;

FIG. 8 is a cross-sectional view of a display device according to an embodiment;

FIG. 9 is a cross-sectional view showing a display device according to an embodiment; and

FIG. 10 is a cross-sectional view showing a display device according to an embodiment.

### DETAILED DESCRIPTION

The subject matter of the present disclosure may have various modifications and may be embodied in different forms, and example embodiments will be explained in more detail with reference to the accompany drawings. The subject matter of the present disclosure may, however, be embodied in different forms and should not be construed as limited to the embodiments set forth herein. Rather, all modifications, equivalents, and substituents which are included in the spirit and technical scope of the present disclosure should be included in the present disclosure.

Like reference numerals refer to like elements throughout. In the drawings, the dimensions of structures may be exaggerated for clarity of illustration. It will be understood that, although the terms first, second, etc. may be used herein to describe various elements, these elements should not be limited by these terms. These terms are only used to distinguish one element from another element. Thus, a first element could be termed a second element without departing from the spirit and scope of the present disclosure. Similarly, a second element could be termed a first element. As used herein, the singular forms are intended to include the plural forms as well, unless the context clearly indicates otherwise.

In the present application, it will be further understood that the terms “comprises” and/or “comprising,” when used in this specification, specify the presence of stated features, numerals, steps, operations, elements, parts, or the combination thereof, but do not preclude the presence or addition of one or more other features, numerals, steps, operations, elements, parts, or the combination thereof.

In the application, when a layer, a film, a region, a plate, etc. is referred to as being “on” or “above” another part, it can be “directly on” the other part, or intervening layers may also be present. On the contrary, when a layer, a film, a region, a plate, etc. is referred to as being “under” or “below” another part, it can be “directly under” the other part, or intervening layers may also be present. Also, when an element is referred to as being “on” another element, it can be under the other element.

In the present description, the term “substituted or unsubstituted” corresponds to substituted or unsubstituted with at least one substituent selected from the group consisting of a deuterium atom, a halogen atom, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, an alkoxy group, a hydrocarbon ring group, an aryl group, and a heterocyclic group. In addition, each of the substituents disclosed may be substituted or unsubstituted. For example, a biphenyl group may be interpreted as an aryl group or a phenyl group substituted with a phenyl group.

In the present description, the term “forming a ring via the combination with an adjacent group” may mean forming a substituted or unsubstituted hydrocarbon ring, or a substituted or unsubstituted heterocycle via the combination with an adjacent group. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocycles or polycycles. In addition, the ring formed via the combination with an adjacent group may be combined with another ring to form a spiro structure.

In the present description, the term “adjacent group” may mean a substituent substituted for an atom which is directly combined with an atom substituted with a corresponding substituent, another substituent substituted for an atom which is substituted with a corresponding substituent, or a substituent sterically positioned at the nearest position to a corresponding substituent. For example, in 1,2-dimethylbenzene, two methyl groups may be interpreted as “adjacent groups” to each other, and in 1,1-diethylcyclopentene, two ethyl groups may be interpreted as “adjacent groups” to each other. In addition, in 4,5-dimethylphenanthrene, two methyl groups may be interpreted as “adjacent groups” to each other.

In the present description, a halogen atom may be a fluorine atom, a chlorine atom, a bromine atom or an iodine atom.

In the present description, an alkyl group may be a linear, branched, or cyclic type (e.g., a linear, branched, or cyclic alkyl group). The carbon number of the alkyl group may be 1 to 50, 1 to 30, 1 to 20, 1 to 10, or 1 to 6. Examples of the alkyl group may include methyl, ethyl, n-propyl, isopropyl, n-butyl, s-butyl, t-butyl, i-butyl, 2-ethylbutyl, 3,3-dimethylbutyl, n-pentyl, i-pentyl, neopentyl, t-pentyl, cyclopentyl, 1-methylpentyl, 3-methylpentyl, 2-ethylpentyl, 4-methyl-2-pentyl, n-hexyl, 1-methylhexyl, 2-ethylhexyl, 2-butylhexyl, cyclohexyl, 4-methylcyclohexyl, 4-t-butylcyclohexyl, n-heptyl, 1-methylheptyl, 2,2-dimethylheptyl, 2-ethylheptyl, 2-butylheptyl, n-octyl, t-octyl, 2-ethyloctyl, 2-butyloctyl, 2-hexyloctyl, 3,7-dimethyloctyl, cyclooctyl, n-nonyl, n-decyl, adamantyl, 2-ethyldecyl, 2-butyldecyl, 2-hexyldecyl, 2-octyldecyl, n-undecyl, n-dodecyl, 2-ethyldodecyl, 2-butyldodecyl, 2-hexyldocecyl, 2-octyldodecyl, n-tridecyl, n-tetradecyl, n-pentadecyl, n-hexadecyl, 2-ethylhexadecyl, 2-butylhexadecyl, 2-hexylhexadecyl, 2-octylhexadecyl, n-heptadecyl, n-octadecyl, n-nonadecyl, n-eicosyl, 2-ethyleicosyl, 2-butyleicosyl, 2-hexyleicosyl, 2-octyleicosyl, n-henicosyl, n-docosyl, n-tricosyl, n-tetracosyl, n-pentacosyl, n-hexacosyl, n-heptacosyl, n-octacosyl, n-nonacosyl, n-triacontyl, etc., without limitation.

In the present description, a cycloalkyl group may mean a ring-type alkyl group (e.g., an alkyl group that forms a ring). The carbon number of the cycloalkyl group may be 3 to 50, 3 to 30, 3 to 20, or 3 to 10. Examples of the cycloalkyl group include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group etc., without limitation.

In the present description, an alkenyl group means a hydrocarbon group including one or more carbon double bonds at a main chain (e.g., in the middle) or at a terminal end (e.g., the terminal) of an alkyl group having a carbon number of 2 or more. The alkenyl group may be a linear chain or a branched chain. The carbon number is not specifically limited, but may be 2 to 30, 2 to 20, or 2 to 10. Examples of the alkenyl group include a vinyl group, an allyl group, a 1-butenyl group, a 1-pentenyl group, a 1,3-butadienyl aryl group, a styrenyl group, a styrylvinyl group, etc., without limitation.

In the present description, an aryl group means an optional functional group or substituent derived from an aromatic hydrocarbon ring. The aryl group may be a monocyclic aryl group or a polycyclic aryl group. The carbon number for forming rings in the aryl group may be 6 to 30, 6 to 20, or 6 to 15. Examples of the aryl group may include phenyl, naphthyl, fluorenyl, anthracenyl, phenanthryl, biphenyl, terphenyl, quaterphenyl, quinquephenyl, sexiphenyl, triphenylenyl, pyrenyl, benzofluoranthenyl, chrysene, etc., without limitation.

In the present description, a fluorenyl group may be substituted, and two substituents may be combined with each other to form a spiro structure. Examples of a substituted fluorenyl group are as follows, but embodiments of the present disclosure are not limited thereto.

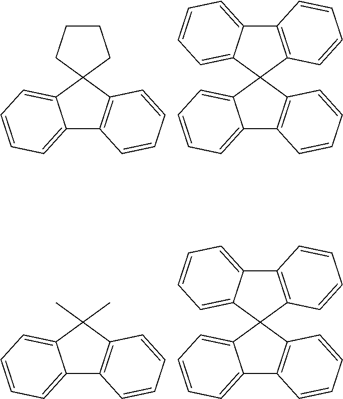

In the present description, a heterocyclic group means an optional functional group or substituent derived from a ring including one or more selected from among B, O, N, P, Si, and S as heteroatoms. The heterocyclic group includes an aliphatic heterocyclic group and an aromatic heterocyclic group. The aromatic heterocyclic group may be a heteroaryl group. The aliphatic heterocyclic group and the aromatic heterocyclic group may be a monocycle or a polycycle.

In the present description, a heteroaryl group may include one or more selected from among B, O, N, P, Si, and S as heteroatoms. If the heteroaryl group includes two or more heteroatoms, two or more heteroatoms may be the same or different. The heteroaryl group may be a monocyclic heterocyclic group or polycyclic heterocyclic group. The carbon number for forming rings of the heteroaryl group may be 2 to 30, 2 to 20, or 2 to 10. Examples of the heteroaryl group may include thiophene, furan, pyrrole, imidazole, pyridine, bipyridine, pyrimidine, triazine, triazole, acridyl, pyridazine, pyrazinyl, quinoline, quinazoline, quinoxaline, phenoxazine, phthalazine, pyrido pyrimidine, pyrido pyrazine, pyrazino pyrazine, isoquinoline, indole, carbazole, N-arylcarbazole, N-heteroarylcarbazole, N-alkylcarbazole, benzoxazole, benzoimidazole, benzothiazole, benzocarbazole, benzothiophene, dibenzothiophene, thienothiophene, benzofuran, phenanthroline, thiazole, isooxazole, oxazole, oxadiazole, thiadiazole, phenothiazine, dibenzosilole, dibenzofuran, etc., without limitation.

In the present description, the same explanation on the above-described aryl group may be applied to an arylene group except that the arylene group is a divalent group. The same explanation on the above-described heteroaryl group may be applied to a heteroarylene group except that the heteroarylene group is a divalent group.

In the present description, a silyl group includes an alkyl silyl group and an aryl silyl group. Examples of the silyl group include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, etc., without limitation.

In the present description, a thio group may include an alkyl thio group and an aryl thio group. The thio group may mean the above-defined alkyl group or aryl group combined with a sulfur atom. Examples of the thio group include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, etc., without limitation.

In the present description, an oxy group may mean the above-defined alkyl group or aryl group which is combined with an oxygen atom. The oxy group may include an alkoxy group and an aryl oxy group. The alkoxy group may be a linear, branched or cyclic chain. The carbon number of the alkoxy group is not specifically limited but may be, for example, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, etc. However, embodiments of the present disclosure are not limited thereto.

In the present description, a boron group may mean the above-defined alkyl group or aryl group combined with a boron atom. The boron group includes an alkyl boron group and an aryl boron group. Examples of the boron group include a dimethylboron group, a diethylboron group, a t-butylboron group, a diphenylboron group, a diphenylboron group, a phenylboron group, and/or the like, without limitation.

In the present description, the carbon number of an amine group is not specifically limited, but may be 1 to 30. The amine group may include an alkyl amine group and an aryl amine group. Examples of the amine group include a methylamine group, a dimethylamine group, a phenylamine group, a diphenylamine group, a naphthylamine group, a 9-methyl-anthracenylamine group, a triphenylamine group, etc., without limitation.

In the present description, a direct linkage may mean a single bond.

In the present description,

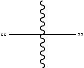

and “-*” mean positions to be connected.

Hereinafter, the light emitting element of an embodiment will be explained referring to the drawings.

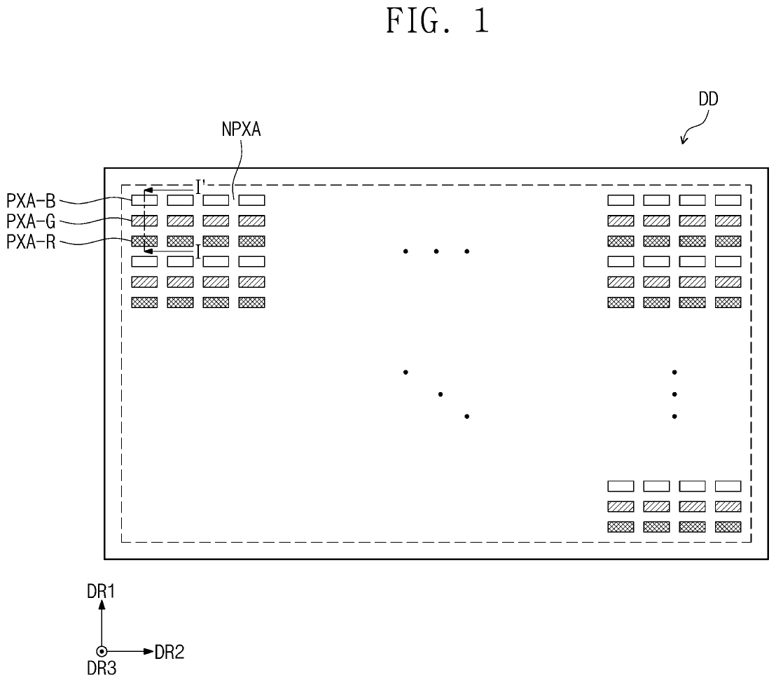

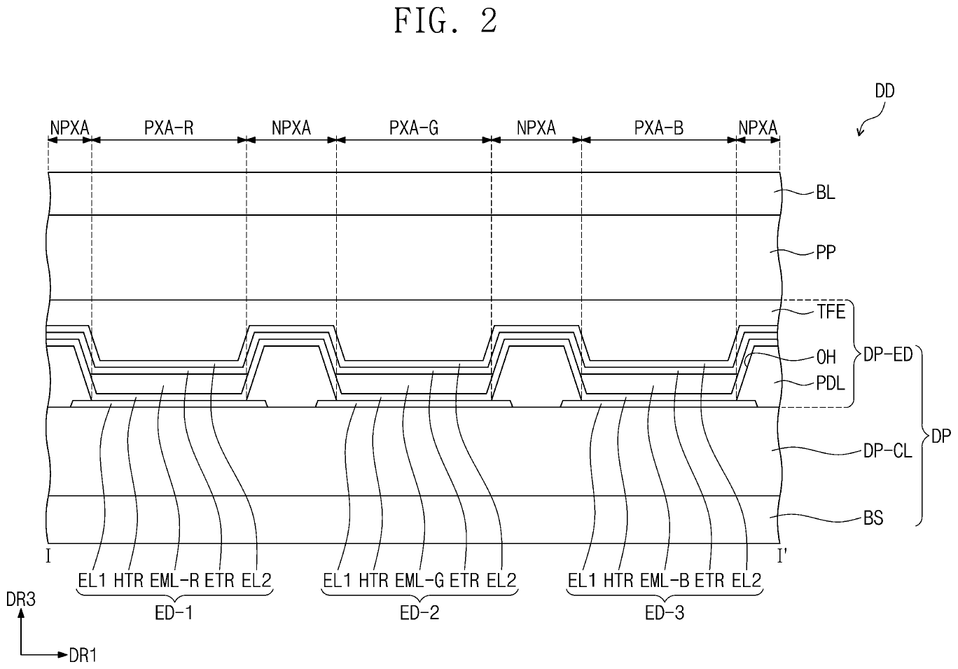

FIG. 1 is a plan view showing an embodiment of a display device DD. FIG. 2 is a cross-sectional view of a display device DD of an embodiment. FIG. 2 is a cross-sectional view showing a part corresponding to line I-I′ of FIG. 1.

The display device DD may include a display panel DP and an optical layer PP on the display panel DP. The display panel DP may include light emitting elements ED-1, ED-2 and ED-3. The display device DD may include a plurality of light emitting elements ED-1, ED-2 and ED-3. The optical layer PP may be on the display panel DP and control reflected light by external light at the display panel DP. The optical layer PP may include, for example, a polarization layer and/or a color filter layer. In some embodiments, the optical layer PP may be omitted from the display device DD.

A base substrate BL may be on the optical layer PP. The base substrate BL may be a member that provides a base surface that the optical layer PP is on. The base substrate BL may be a glass substrate, a metal substrate, a plastic substrate, etc. However, embodiments of the present disclosure are not limited thereto, and the base substrate BL may be an inorganic layer, an organic layer or a composite material layer. In some embodiments, the base substrate BL may be omitted.

The display device DD according to an embodiment may further include a plugging layer. The plugging layer may be between a display element layer DP-ED and a base substrate BL. The plugging layer may be an organic layer. The plugging layer may include at least one selected from among an acrylic resin, a silicon-based resin and an epoxy-based resin.

The display panel DP may include a base layer BS, a circuit layer DP-CL on the base layer BS and a display element layer DP-ED. The display element layer DP-ED may include a pixel definition layer PDL, light emitting elements ED-1, ED-2 and ED-3 in the pixel definition layer PDL, and an encapsulating layer TFE on the light emitting elements ED-1, ED-2 and ED-3.

The base layer BS may be a member that provides a base surface that the display element layer DP-ED is on. The base layer BS may be a glass substrate, a metal substrate, a plastic substrate, etc. However, embodiments of the present disclosure are not limited thereto, and the base layer BS may be an inorganic layer, an organic layer or a composite material layer.

In an embodiment, the circuit layer DP-CL is on the base layer BS, and the circuit layer DP-CL may include a plurality of transistors. Each of the transistors may include a control electrode, an input electrode, and an output electrode. For example, the circuit layer DP-CL may include switching transistors and driving transistors for driving the light emitting elements ED-1, ED-2 and ED-3 of the display element layer DP-ED.

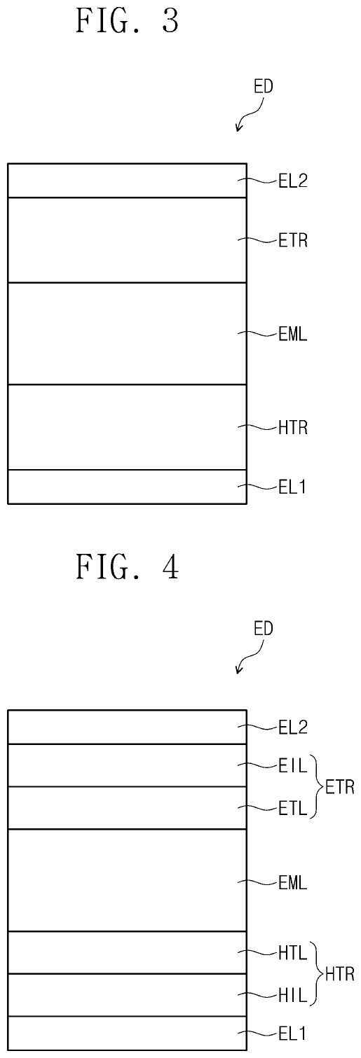

The light emitting elements ED-1, ED-2 and ED-3 may have the structures of the light emitting elements ED of embodiments according to FIG. 3 to FIG. 6, which will be further explained herein below. The light emitting elements ED-1, ED-2 and ED-3 may include a first electrode EL1, a hole transport region HTR, emission layers EML-R, EML-G and EML-B, an electron transport region ETR and a second electrode EL2.

In FIG. 2, shown is an embodiment where the emission layers EML-R, EML-G and EML-B of light emitting elements ED-1, ED-2 and ED-3 are in opening portions OH defined in a pixel definition layer PDL, and a hole transport region HTR, an electron transport region ETR and a second electrode EL2 are provided as common layers in all light emitting elements ED-1, ED-2 and ED-3. However, embodiments of the present disclosure are not limited thereto. Different from FIG. 2, in an embodiment, the hole transport region HTR and the electron transport region ETR may be patterned and provided in the opening portions OH defined in the pixel definition layer PDL. For example, in an embodiment, the hole transport region HTR, the emission layers EML-R, EML-G and EML-B, and the electron transport region ETR of the light emitting elements ED-1, ED-2 and ED-3 may be patterned by an ink jet printing method and provided.

An encapsulating layer TFE may cover the light emitting elements ED-1, ED-2 and ED-3. The encapsulating layer TFE may encapsulate the display element layer DP-ED. The encapsulating layer TFE may be a thin film encapsulating layer. The encapsulating layer TFE may be one layer or a stacked layer of a plurality of layers. The encapsulating layer TFE includes at least one insulating layer. The encapsulating layer TFE according to an embodiment may include at least one inorganic layer (hereinafter, encapsulating inorganic layer). In addition, the encapsulating layer TFE according to an embodiment may include at least one organic layer (hereinafter, encapsulating organic layer) and at least one encapsulating inorganic layer.

The encapsulating inorganic layer protects the display element layer DP-ED from moisture/oxygen, and the encapsulating organic layer protects the display element layer DP-ED from foreign materials such as dust particles. The encapsulating inorganic layer may include silicon nitride, silicon oxy nitride, silicon oxide, titanium oxide, and/or aluminum oxide, without specific limitation. The encapsulating organic layer may include an acrylic compound, an epoxy-based compound, etc. The encapsulating organic layer may include a photopolymerizable organic material, without specific limitation.

The encapsulating layer TFE may be on the second electrode EL2 and may be provided while filling the opening portion OH.

Referring to FIG. 1 and FIG. 2, the display device DD may include a non-luminous area NPXA and luminous areas PXA-R, PXA-G and PXA-B. The luminous areas PXA-R, PXA-G and PXA-B may be areas emitting light produced from the light emitting elements ED-1, ED-2 and ED-3, respectively. The luminous areas PXA-R, PXA-G and PXA-B may be separated (e.g., spaced apart) from each other on a plane.

The luminous areas PXA-R, PXA-G and PXA-B may be areas separated by the pixel definition layer PDL. The non-luminous areas NPXA may be areas between neighboring luminous areas PXA-R, PXA-G and PXA-B and may be areas corresponding to the pixel definition layer PDL. In the present disclosure, each of the luminous areas PXA-R, PXA-G and PXA-B may correspond to each pixel. The pixel definition layer PDL may divide the light emitting elements ED-1, ED-2 and ED-3. The emission layers EML-R, EML-G and EML-B of the light emitting elements ED-1, ED-2 and ED-3 may be provided and divided in the opening portions OH defined in the pixel definition layer PDL.

The luminous areas PXA-R, PXA-G and PXA-B may be divided into a plurality of groups according to the color of light produced from the light emitting elements ED-1, ED-2 and ED-3. In the display device DD of an embodiment, shown in FIG. 1 and FIG. 2, three luminous areas PXA-R, PXA-G and PXA-B that emit red light, green light and blue light are illustrated as an embodiment. For example, the display device DD of an embodiment may include a red luminous area PXA-R, a green luminous area PXA-G and a blue luminous area PXA-B, which are separated (e.g., spaced apart) from each other.

In the display device DD according to an embodiment, a plurality of light emitting elements ED-1, ED-2 and ED-3 may emit light having different wavelength regions. For example, in an embodiment, the display device DD may include a first light emitting element ED-1 emitting red light, a second light emitting element ED-2 emitting green light, and a third light emitting element ED-3 emitting blue light. In some embodiments, each of the red luminous area PXA-R, the green luminous area PXA-G, and the blue luminous area PXA-B of the display device DD may correspond to the first light emitting element ED-1, the second light emitting element ED-2, and the third light emitting element ED-3.

However, embodiments of the present disclosure are not limited thereto, and the first to third light emitting elements ED-1, ED-2 and ED-3 may emit light in the same wavelength region, or at least one thereof may emit light in a different wavelength region. For example, all the first to third light emitting elements ED-1, ED-2 and ED-3 may emit blue light.

The luminous areas PXA-R, PXA-G and PXA-B in the display device DD according to an embodiment may be arranged in a stripe shape. Referring to FIG. 1, a plurality of red luminous areas PXA-R, a plurality of green luminous areas PXA-G and a plurality of blue luminous areas PXA-B may be arranged along a second directional axis DR2. In addition, the red luminous area PXA-R, the green luminous area PXA-G and the blue luminous area PXA-B may be arranged by turns (e.g., alternately arranged) along a first directional axis DR1.

In FIG. 1 and FIG. 2, the areas of the luminous areas PXA-R, PXA-G and PXA-B are shown to be similar, but embodiments of the present disclosure are not limited thereto. The areas of the luminous areas PXA-R, PXA-G and PXA-B may be different from each other according to the wavelength region of light emitted. According to embodiments of the present disclosure, the areas of the luminous areas PXA-R, PXA-G and PXA-B may mean areas on a plane defined by the first directional axis DR1 and the second directional axis DR2.

The arrangement type of the luminous areas PXA-R, PXA-G and PXA-B is not limited to the configuration shown in FIG. 1, and the arrangement order of the red luminous areas PXA-R, the green luminous areas PXA-G and the blue luminous areas PXA-B may be provided in various suitable combinations according to the properties of display quality required or desired for the display device DD. For example, the arrangement type of the luminous areas PXA-R, PXA-G and PXA-B may be a PENTILE® arrangement type (e.g., an RGBG matrix, RGBG structure, or RGBG matrix structure), or a DIAMOND PIXEL™) arrangement type. PENTILE® is a duly registered trademark of Samsung Display Co., Ltd.

In addition, the areas of the luminous areas PXA-R, PXA-G and PXA-B may be different from each other. For example, in an embodiment, the area of the green luminous area PXA-G may be smaller than the area of the blue luminous area PXA-B, but embodiments of the present disclosure are not limited thereto.

Hereinafter, FIG. 3 to FIG. 6 are cross-sectional views schematically showing light emitting elements according to embodiments. The light emitting element ED according to an embodiment may include a first electrode EL1, a second electrode EL2 opposite to the first electrode EL1, and at least one functional layer between the first electrode EL1 and the second electrode EL2. The light emitting element ED of an embodiment may include an amine compound of an embodiment, which will be further explained herein below, in the at least one functional layer.

The light emitting element ED may include a hole transport region HTR, an emission layer EML, an electron transport region ETR, and/or the like, stacked in order, as the at least one functional layer. Referring to FIG. 3, the light emitting element ED of an embodiment may include a first electrode EL1, a hole transport region HTR, an emission layer EML, an electron transport region ETR, and a second electrode EL2, stacked in order.

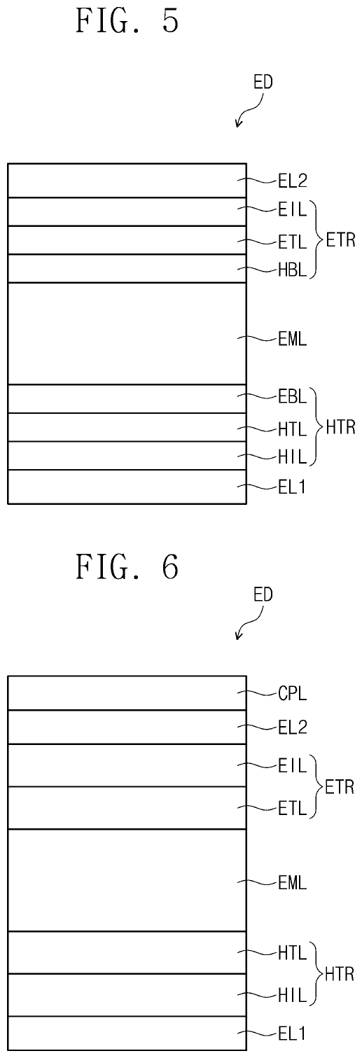

When compared to FIG. 3, FIG. 4 shows the cross-sectional view of a light emitting element ED of an embodiment, wherein a hole transport region HTR includes a hole injection layer HIL and a hole transport layer HTL, and an electron transport region ETR includes an electron injection layer EIL and an electron transport layer ETL. In addition, when compared to FIG. 3, FIG. 5 shows the cross-sectional view of a light emitting element ED of an embodiment, wherein a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. When compared to FIG. 4, FIG. 6 shows the cross-sectional view of a light emitting element ED of an embodiment, including a capping layer CPL on the second electrode EL2.

The light emitting element ED of an embodiment may include an amine compound of an embodiment, which will be further explained herein below, in a hole transport region HTR. The light emitting element ED of an embodiment may include an amine compound of an embodiment in at least one selected from among the hole injection layer HIL, hole transport layer HTL and electron blocking layer EBL of the hole transport region HTR. For example, in the light emitting element ED of an embodiment, the hole transport layer HTL may include the amine compound of an embodiment.

In the light emitting element ED according to an embodiment, the first electrode EL1 has conductivity (e.g., electrical conductivity). The first electrode EL1 may be formed using a metal material, a metal alloy and/or a conductive compound (e.g., an electrically conductive compound). The first electrode EL1 may be an anode or a cathode. However, embodiments of the present disclosure are not limited thereto. In addition, the first electrode EL1 may be a pixel electrode. The first electrode EL1 may be a transmissive electrode, a transflective electrode, or a reflective electrode. The first electrode EL1 may include at least one selected from among Ag, Mg, Cu, Al, Pt, Pd, Au, Ni, Nd, Ir, Cr, Li, Ca, LiF, Mo, Ti, W, In, Sn and Zn, compounds of two or more selected therefrom, mixtures of two or more selected therefrom, or oxides thereof.

If the first electrode EL1 is the transmissive electrode, the first electrode EL1 may include a transparent metal oxide such as indium tin oxide (ITO), indium zinc oxide (IZO), zinc oxide (ZnO), and indium tin zinc oxide (ITZO). If the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, compounds thereof, and/or mixtures thereof (for example, a mixture of Ag and Mg). Also, the first electrode EL1 may have a structure including a plurality of layers including a reflective layer or a transflective layer formed using the above materials, and a transmissive conductive layer formed using ITO, IZO, ZnO, and/or ITZO. For example, the first electrode EL1 may have a three-layer structure of ITO/Ag/ITO. However, embodiments of the present disclosure are not limited thereto. The first electrode EL1 may include the above-described metal materials, combinations of two or more metal materials selected from the above-described metal materials, and/or oxides of the above-described metal materials. The thickness of the first electrode EL1 may be from about 700 Å to about 10,000 Å. For example, the thickness of the first electrode EL1 may be from about 1,000 Å to about 3,000 Å.

The hole transport region HTR is provided on the first electrode EL1. The hole transport region HTR may have a single layer formed using a single material, a single layer formed using a plurality of different materials, or a multilayer structure including a plurality of layers formed using a plurality of different materials.

The hole transport region HTR may include at least one selected from a hole injection layer HIL, a hole transport layer HTL, and an electron blocking layer EBL. In some embodiments, the hole transport region HTR may include a plurality of hole transport layers stacked.

In addition, otherwise, the hole transport region HTR may have the structure of a single layer of a hole injection layer HIL or a hole transport layer HTL, and may have a structure of a single layer formed using a hole injection material and a hole transport material. In an embodiment, the hole transport region HTR may have a structure of a single layer formed using a plurality of different materials, or a structure stacked from the first electrode EL1 of hole injection layer HIL/hole transport layer HTL, hole injection layer HIL/hole transport layer HTL/buffer layer, hole injection layer HIL/buffer layer, or hole transport layer HTL/buffer layer, without limitation.

The thickness of the hole transport region HTR may be, for example, about 50 Å to about 15,000 Å. The hole transport region HTR may be formed using various suitable methods such as a vacuum deposition method, a spin coating method, a cast method, a Langmuir-Blodgett (LB) method, an inkjet printing method, a laser printing method, and/or a laser induced thermal imaging (LITI) method.

The light emitting element ED of an embodiment may include the amine compound of an embodiment in a hole transport region HTR. In the light emitting element ED of an embodiment, the hole transport region HTR may include an electron injection layer EIL, and a hole transport layer HTL, and the hole transport layer HTL may include the amine compound of an embodiment. The amine compound of an embodiment may be included in a layer adjacent to an emission layer EML among the layers included in the hole transport region HTR.

The amine compound of an embodiment includes a structure of a core nitrogen atom connected with a first substituent, a second substituent and a third substituent. The amine compound of an embodiment includes an amine group, and the first substituent to the third substituent may be combined with the amine group and the amine compound of an embodiment. In an embodiment, the first substituent essentially includes a naphthalene moiety. An optional carbon atom of the naphthalene moiety is connected with the amine group, and a carbon atom adjacent to the carbon atom is connected with an alkyl group. For example, if a carbon atom at position 1 of the naphthalene moiety is connected with the amine group, a carbon atom at position 2 may be connected with the alkyl group. Otherwise, if the carbon atom at position 2 of the naphthalene moiety is connected with the amine group, at least one selected from the carbon atom at position 1 and a carbon atom at position 3 may be connected with the alkyl group. The second substituent may include a fluorenyl moiety, and the third substituent may be an alkyl group, an alkenyl group, an alkynyl group, an aryl group, or a heteroaryl group. The amine compound of an embodiment may be a monoamine compound including a single number of the amine group.

In an embodiment, the amine compound may be represented by Formula 1 below.

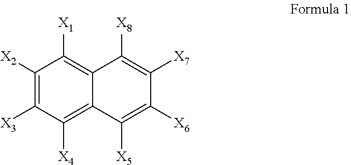

In Formula 1, X₁ is a hydrogen atom, a deuterium atom, a halogen atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or represented by Formula 2 below. X₂ is a substituted or unsubstituted alkyl group of 1 to 4 carbon atoms, or represented by Formula 2 below. For example, X₁ may be a hydrogen atom, a deuterium atom, a substituted or unsubstituted methyl group, or represented by Formula 2. For example, X₂ may be a substituted or unsubstituted methyl group, or represented by Formula 2.

In Formula 1, X₃ to X₈ are each independently a hydrogen atom, a deuterium atom, a halogen atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms. For example, X₃ to X₈ may be each independently a hydrogen atom, or a deuterium atom.

In Formula 1, any one selected from among X₁ and X₂ is represented by Formula 2. In some embodiments, at a position adjacent to the substituent represented by Formula 2 among X₁ and X₂, an alkyl group of 1 to 4 carbon atoms is connected. In an embodiment, if X₁ is represented by Formula 2, X₂ is a substituted or unsubstituted alkyl group of 1 to 4 carbon atoms. If X₂ is represented by Formula 2, any one selected from among X₁ and X₃ is a substituted or unsubstituted alkyl group of 1 to 4 carbon atoms. For example, if X₁ is represented by Formula 2, X₂ may be a substituted or unsubstituted methyl group. For example, if X₂ is represented by Formula 2, any one selected from among X₁ and X₃ may be a substituted or unsubstituted methyl group.

According to embodiments of the present disclosure, the naphthalene moiety represented by Formula 1 may correspond to the above-described first substituent.

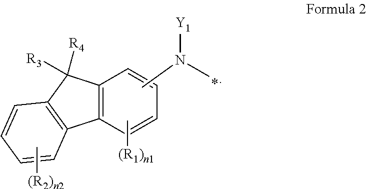

In Formula 2, R₁ to R₄ are each independently a hydrogen atom, a deuterium atom, a halogen atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms. For example, R₁, R₃ and R₄ may be each independently a hydrogen atom or a deuterium atom. For example, R₂ may be a hydrogen atom, a substituted or unsubstituted alkyl group of 1 to 10 carbon atoms, or a substituted or unsubstituted aryl group of 6 to 12 ring-forming carbon atoms.

In Formula 2, Y₁ is a substituted or unsubstituted alkyl group of 1 to 20 carbon atoms, a substituted or unsubstituted alkenyl group of 2 to 20 carbon atoms, a substituted or unsubstituted alkynyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms. For example, Y₁ is a substituted or unsubstituted phenyl group, a substituted or unsubstituted naphthyl group, a substituted or unsubstituted fluorene group or a substituted or unsubstituted carbazole group.

In Formula 2, n1 is an integer of 0 to 3. If n1 is 0, the amine compound of an embodiment may mean one unsubstituted with R₁ (e.g., the carbon atoms that could otherwise be substituted with R₁ are bonded to hydrogen). A case where n1 is 3, and all R₁ are hydrogen atoms, may be the same as a case where n1 is 0. If n1 is an integer of 2 or more, a plurality of R₁ may be all the same, or at least one among a plurality of R₁ may be different.

In Formula 2, n2 is an integer of 0 to 4. If n2 is 0, the amine compound of an embodiment may mean one unsubstituted with R₂. A case where n2 is 4, and all R₂ are hydrogen atoms, may be the same as a case where n2 is 0. If n2 is an integer of 2 or more, a plurality of R₂ may be all the same, or at least one among a plurality of R₂ may be different.

In some embodiments, in Formula 2, among the substituents of the core nitrogen atom, a substituent including a fluorenyl moiety may correspond to the above-explained second substituent, and Y₁ may correspond to the above-explained third substituent.

In an embodiment, the amine compound may be represented by any one selected from among Formula 1-1 to Formula 1-3.

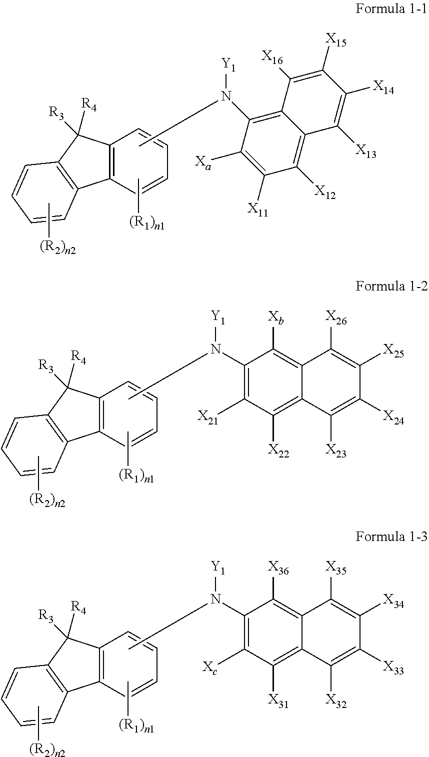

Formula 1-1 to Formula 1-3 represent cases of Formula 1 where any one selected from among X₁ and X₂ is represented by Formula 2, and the position where the alkyl group is substituted is specified. Formula 1-1 represents a case of Formula 1 where X₁ is represented by Formula 2, and Formula 1-2 and Formula 1-3 represent Formula 1 where X₂ is represented by Formula 2. Formula 1-2 represents a case of Formula 1 where X₂ is represented by Formula 2, and X₁ is essentially substituted with an alkyl group, and Formula 1-3 represents a case of Formula 1 where X₂ is represented by Formula 2, and X₃ is essentially substituted with an alkyl group.

In Formula 1-1 to Formula 1-3, Xₐ, X_{b} and X_{c} are each independently a substituted or unsubstituted alkyl group of 1 to 4 carbon atoms. For example, Xₐ, X_{b} and X_{c} may be each independently an unsubstituted methyl group.

In Formula 1-1 to Formula 1-3, X₁₁ to X₃₆ are each independently a hydrogen atom, a deuterium atom, a halogen atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms. For example, X₁₁ to X₃₆ may be each independently a hydrogen atom or a deuterium atom.

According to embodiments of the disclosure, in Formula 1-1 to Formula 1-3, the same contents explained with respect to Formula 2 may be applied for R₁ to R₄, n1, n2 and Y₁.

In an embodiment, the substituent represented by Formula 2 in the amine compound may be represented by Formula 2-1.

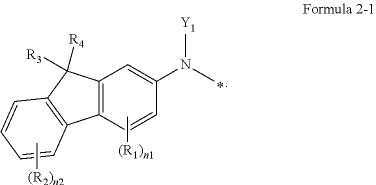

Formula 2-1 represents a case of Formula 2 where the position of the substituent including a fluorenyl moiety bonded to the core nitrogen atom of the amine group is specified.

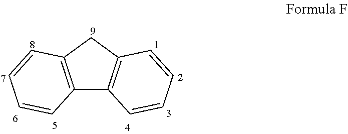 [SMILES: c1ccc2c(c1)Cc1ccccc1-2]

According to embodiments of the disclosure, the number of carbon constituting the fluorenyl moiety is shown in Formula F. Formula 2-1 represents a structure where carbon at position 2 of the fluorenyl moiety is connected with the amine group.

According to embodiments of the disclosure, in Formula 2-1, the same contents explained with respect to Formula 2 may be applied for R₁ to R₄, n1, n2 and Y₁.

In some embodiments, the substituent represented by Formula 2 in the amine compound may be represented by Formula 2-2.

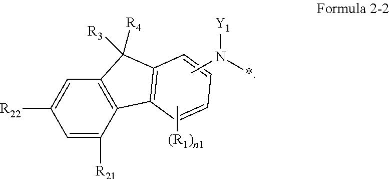

Formula 2-2 represents a case of Formula 2 where n2 is 2, and two R₂ groups of Formula 2 are provided. In some embodiments, Formula 2-2 may have a structure where carbon at positions 5 and 7 of the fluorenyl moiety are connected with the R₂ groups of Formula 2, respectively, carbon at positions 6 and 8 of the fluorenyl moiety are connected with hydrogen atoms, respectively, and carbon at one position selected from among 1 to 4 of the fluorenyl moiety is connected with the amine group of the amine compound.

In Formula 2-2, R₂₁ and R₂₂ are each independently a hydrogen atom, a substituted or unsubstituted alkyl group of 1 to 20 carbon atoms, or a substituted or unsubstituted aryl group of 6 to 30 ring-forming carbon atoms. At least one selected from among R₂₁ and R₂₂ may be a substituted or unsubstituted alkyl group of 1 to 20 carbon atoms, or a substituted or unsubstituted aryl group of 6 to 30 ring-forming carbon atoms. For example, R₂₁ may be a substituted or unsubstituted methyl group or a substituted or unsubstituted phenyl group. For example, R₂₂ may be a hydrogen atom or a substituted or unsubstituted methyl group.

In Formula 2-2, the same contents explained with respect to Formula 2 may be applied for R₁, R₃, R₄, n1 and Y₁.

In an embodiment, the substituent represented by Formula 2 in the amine compound may be represented by Formula 2-3.

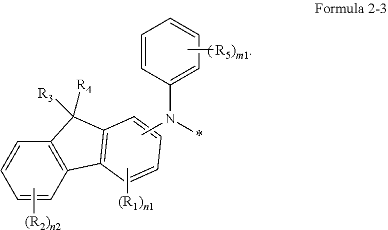

Formula 2-3 represents a case of Formula 2 where Y₁ is specified. Formula 2-3 represents a case where Y₁ is a substituted or unsubstituted phenyl group. The amine compound in which the substituent represented by Formula 2 is represented by Formula 2-3, may be a triarylamine compound.

In Formula 2-3, R₅ is a hydrogen atom, a deuterium atom, a halogen atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms. In some embodiments, R₅ may be combined with an adjacent group to form a ring. For example, R₅ may be a substituted or unsubstituted phenyl group or a substituted or unsubstituted naphthyl group. In some embodiments, a plurality of R₅ may be combined with each other to form a ring. For example, one R₅ may be provided as a phenyl group, another one R₅ may be provided as an amine group, and these may be combined with each other to form a carbazole moiety. For example, one R₅ may be a phenyl group, another one R₅ may be provided as a methyl group, and these may be combined with each other to form a fluorenyl moiety.

For example, R₅ may be represented by any one selected from among A1-1 to A1-8. In A1-1 to A1-8, “-*” is a position connected with the phenyl group of Formula 2.

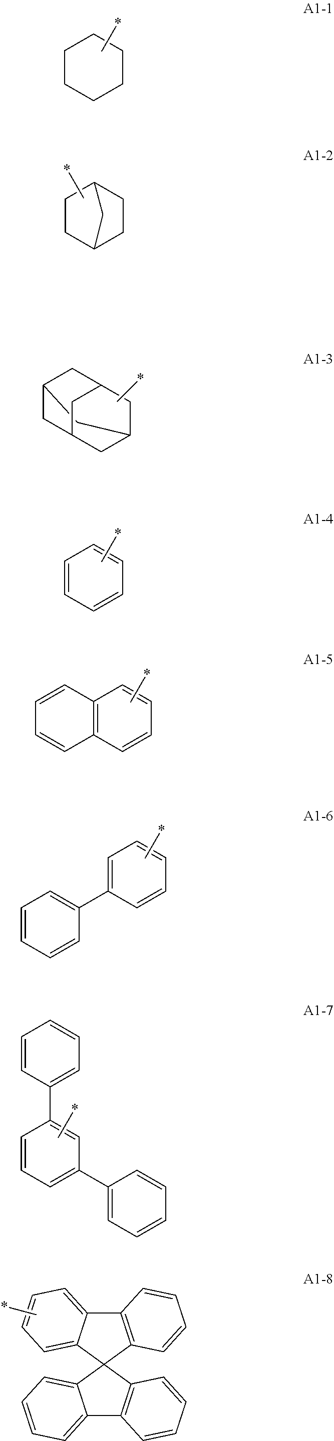

In Formula 2-3, m1 is an integer of 0 to 5. If m1 is 0, the amine compound of an embodiment may mean one unsubstituted with R₅ (e.g., each of the carbon atoms that could otherwise be substituted with R₅ is bonded to hydrogen). A case where m1 is 5, and each of R₅ is a hydrogen atom, may be the same as a case where m1 is 0. If m1 is an integer of 2 or more, a plurality of R₅ may be all the same, or at least one among a plurality of R₅ may be different.

According to embodiments of the disclosure, in Formula 2-3, the same contents explained with respect to Formula 2 may be applied for R₁ to R₄, n1 and n2.

The amine compound of an embodiment may be represented by any one selected from among the compounds in Compound Group 1. The hole transport region HTR of the light emitting element ED of an embodiment may include at least one selected from among the amine compounds shown in Compound Group 1. For example, the hole transport layer HTL of the light emitting element ED may include at least one selected from among the amine compounds shown in Compound Group 1.

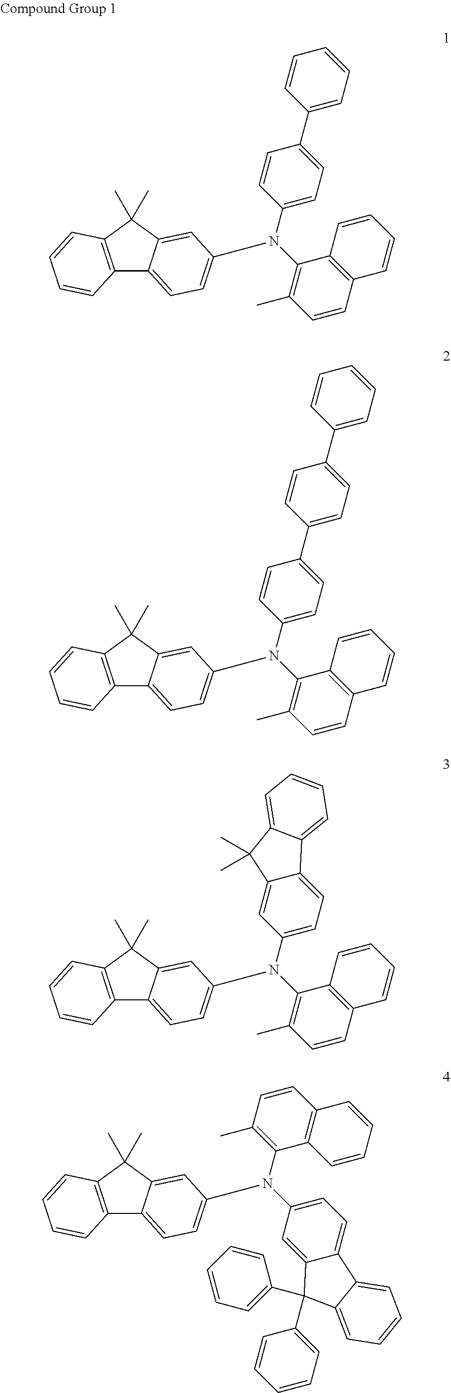

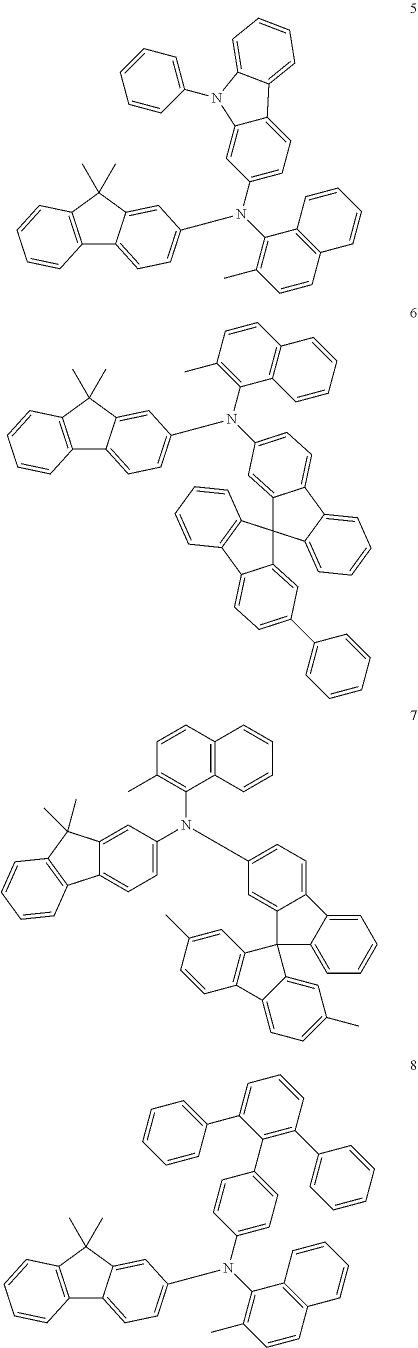

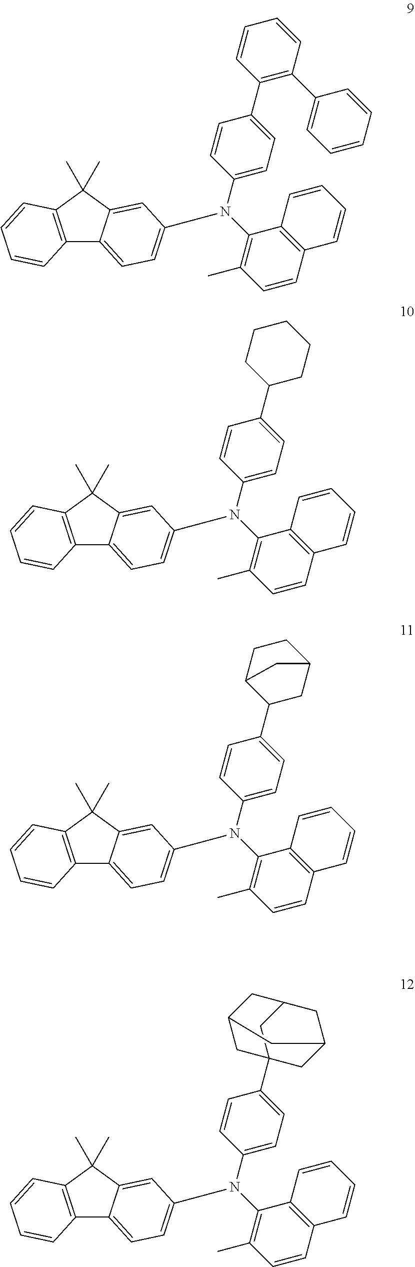

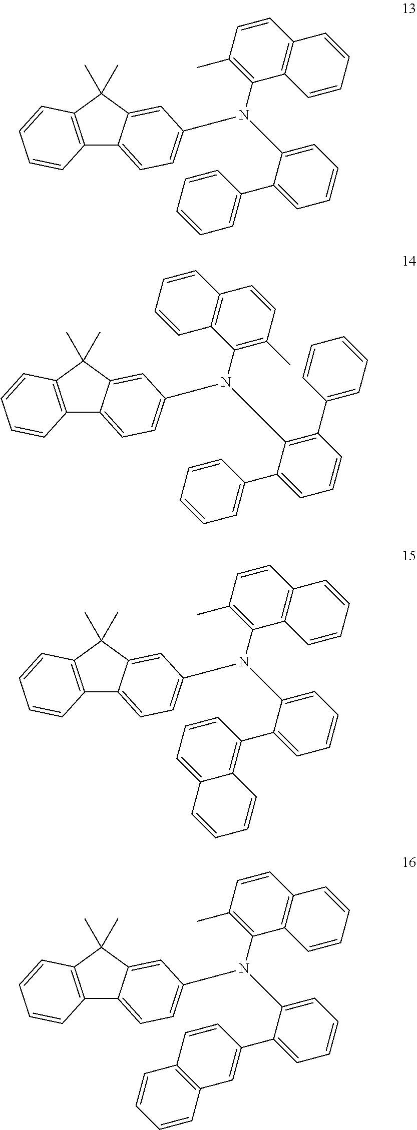

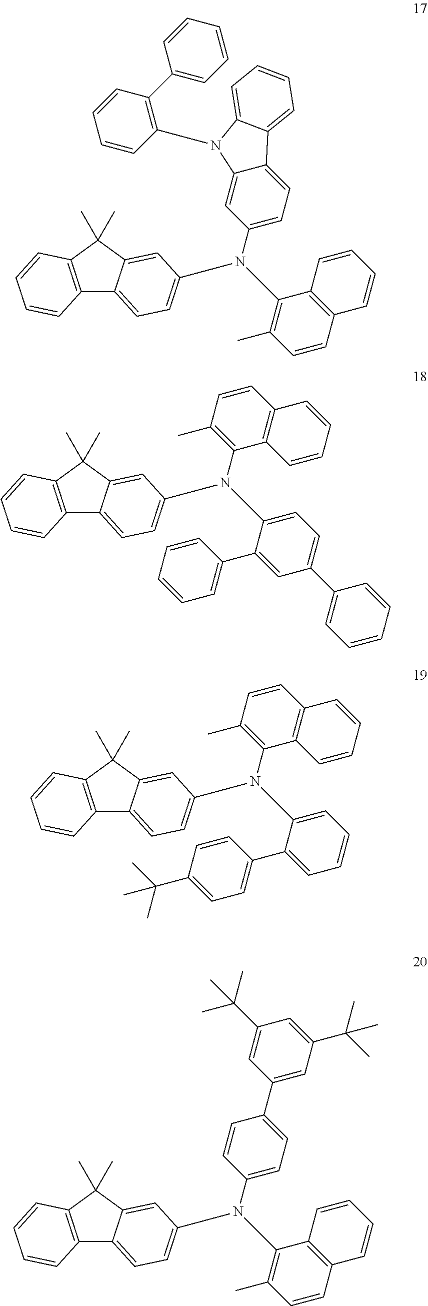 [SMILES: Cc1ccc2ccccc2c1N(c1ccc2c(c1)C(C)(C)c1ccccc1-2)c1ccc2c3ccccc3n(-c3ccccc3-c3ccccc3)c2c1 ; Cc1ccc2ccccc2c1N(c1ccc2c(c1)C(C)(C)c1ccccc1-2)c1ccc(-c2ccccc2)cc1-c1ccccc1 ; Cc1ccc2ccccc2c1N(c1ccc2c(c1)C(C)(C)c1ccccc1-2)c1ccccc1-c1ccc(C(C)(C)C)cc1 ; Cc1ccc2ccccc2c1N(c1ccc(-c2cc(C(C)(C)C)cc(C(C)(C)C)c2)cc1)c1ccc2c(c1)C(C)(C)c1ccccc1-2]

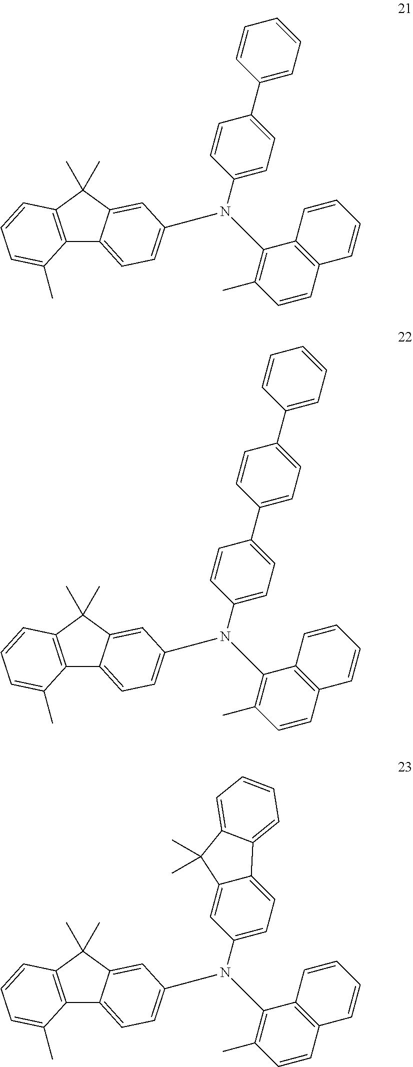 [SMILES: Cc1cccc2c1-c1ccc(N(c3ccc(-c4ccccc4)cc3)c3c(C)ccc4ccccc34)cc1C2(C)C ; Cc1cccc2c1-c1ccc(N(c3ccc(-c4ccc(-c5ccccc5)cc4)cc3)c3c(C)ccc4ccccc34)cc1C2(C)C ; Cc1cccc2c1-c1ccc(N(c3ccc4c(c3)C(C)(C)c3ccccc3-4)c3c(C)ccc4ccccc34)cc1C2(C)C]

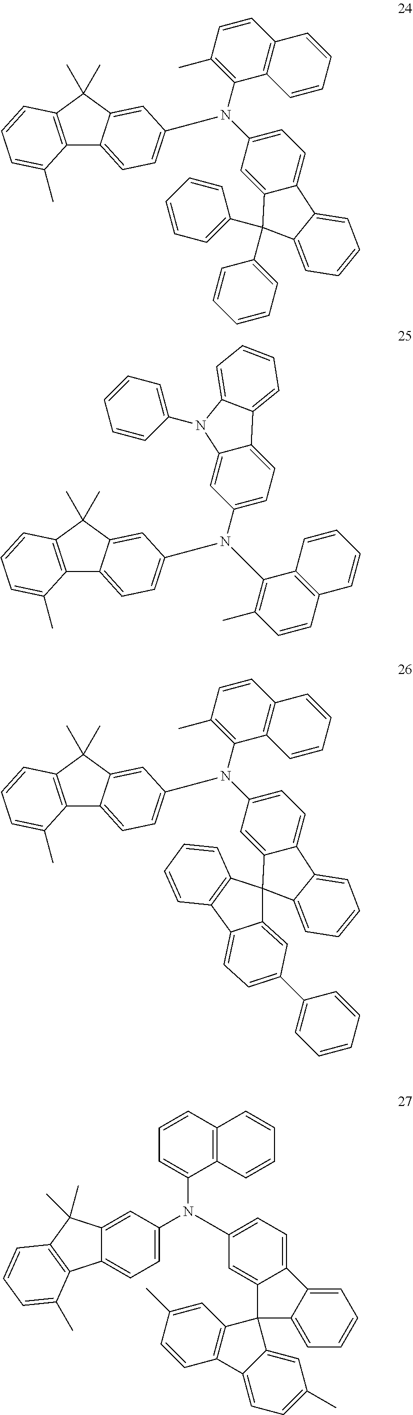

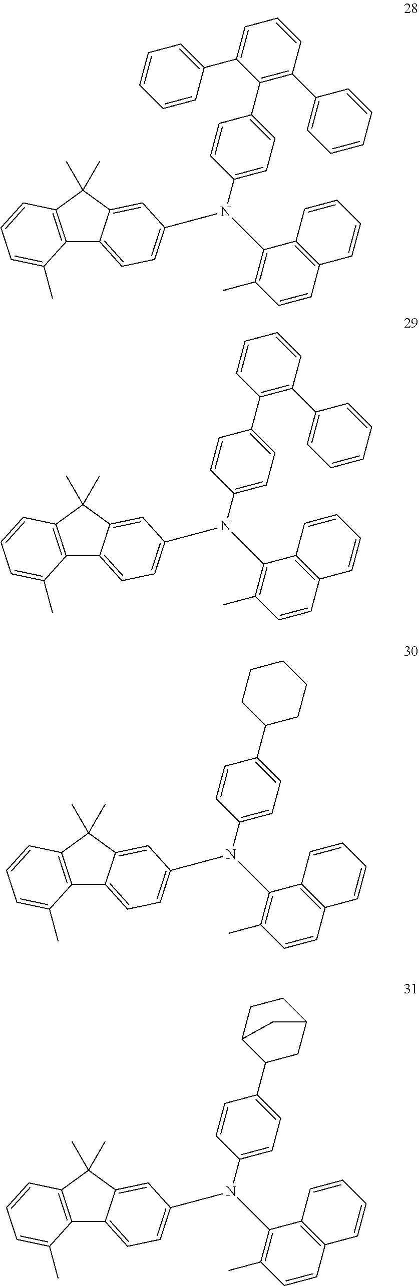

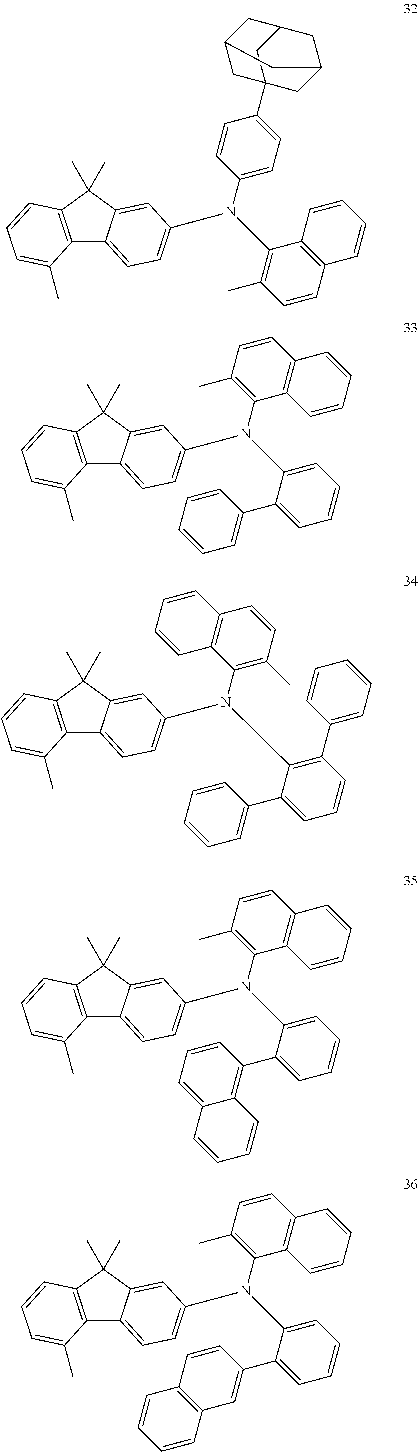

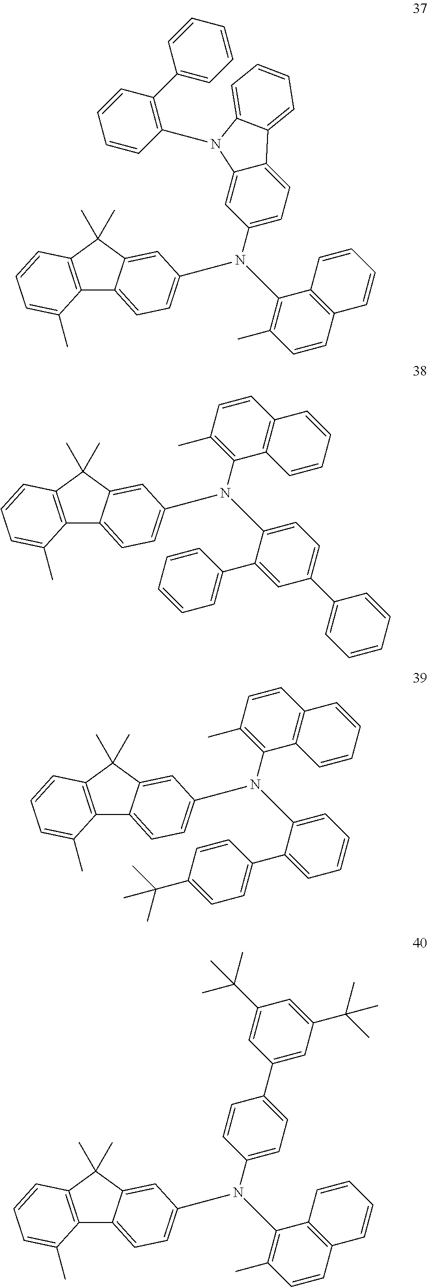 [SMILES: Cc1cccc2c1-c1ccc(N(c3ccc4c5ccccc5n(-c5ccccc5-c5ccccc5)c4c3)c3c(C)ccc4ccccc34)cc1C2(C)C ; Cc1cccc2c1-c1ccc(N(c3ccc(-c4ccccc4)cc3-c3ccccc3)c3c(C)ccc4ccccc34)cc1C2(C)C ; Cc1cccc2c1-c1ccc(N(c3ccccc3-c3ccc(C(C)(C)C)cc3)c3c(C)ccc4ccccc34)cc1C2(C)C ; Cc1cccc2c1-c1ccc(N(c3ccc(-c4cc(C(C)(C)C)cc(C(C)(C)C)c4)cc3)c3c(C)ccc4ccccc34)cc1C2(C)C]

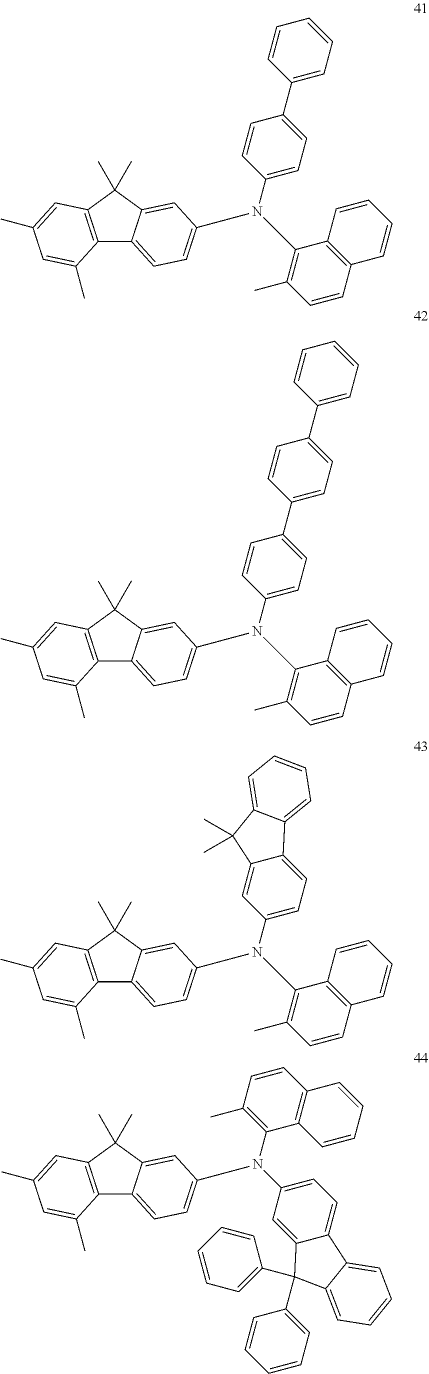

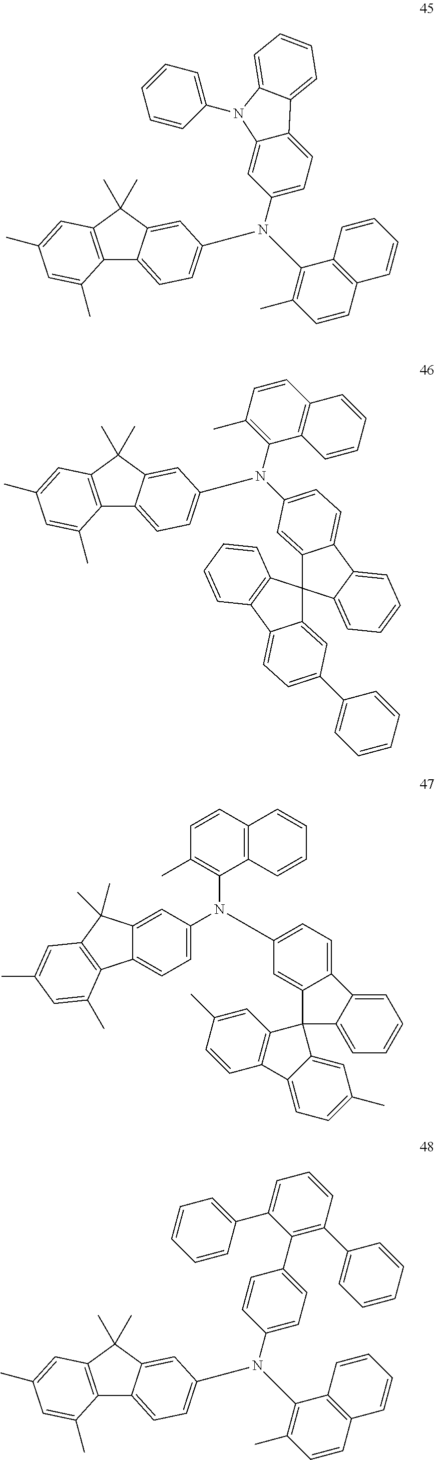 [SMILES: Cc1cc(C)c2c(c1)C(C)(C)c1cc(N(c3ccc4c5ccccc5n(-c5ccccc5)c4c3)c3c(C)ccc4ccccc34)ccc1-2 ; Cc1cc(C)c2c(c1)C(C)(C)c1cc(N(c3ccc4c(c3)C3(c5ccccc5-c5ccc(-c6ccccc6)cc53)c3ccccc3-4)c3c(C)ccc4ccccc34)ccc1-2 ; Cc1cc(C)c2c(c1)C(C)(C)c1cc(N(c3ccc4c(c3)C3(c5ccccc5-4)c4cc(C)ccc4-c4ccc(C)cc43)c3c(C)ccc4ccccc34)ccc1-2 ; Cc1cc(C)c2c(c1)C(C)(C)c1cc(N(c3ccc(-c4c(-c5ccccc5)cccc4-c4ccccc4)cc3)c3c(C)ccc4ccccc34)ccc1-2]

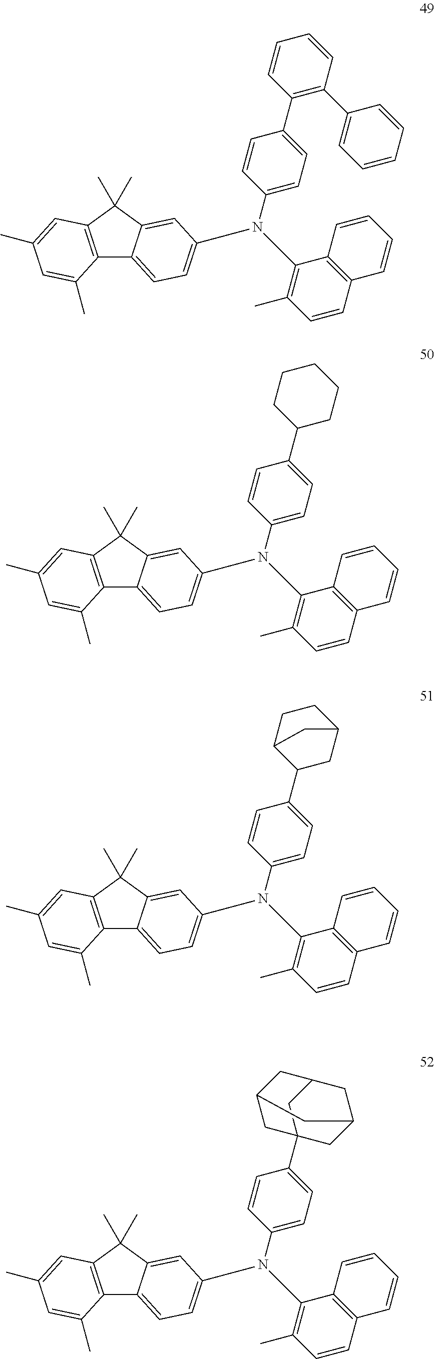

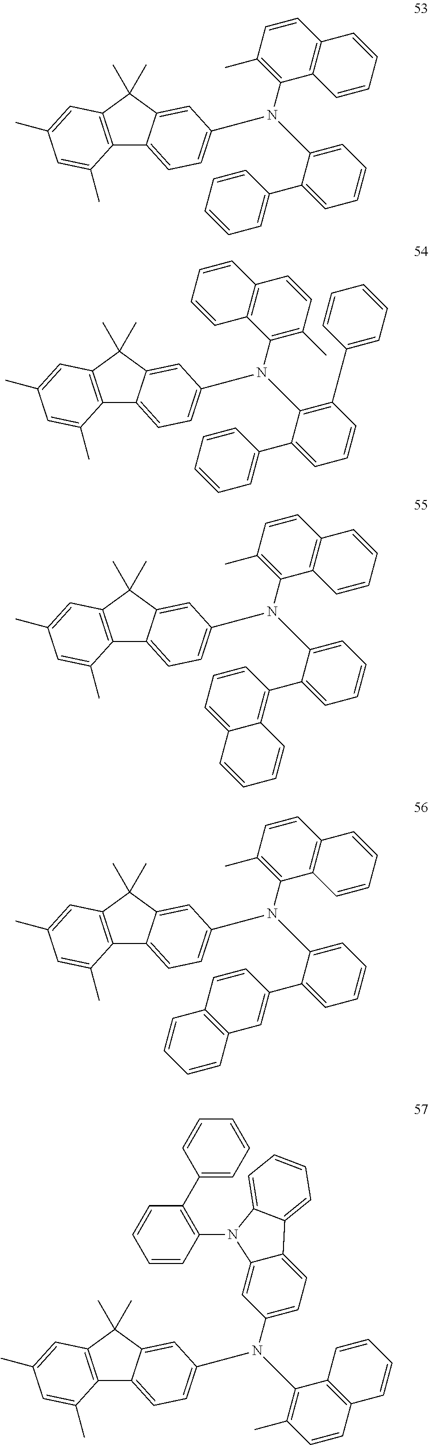

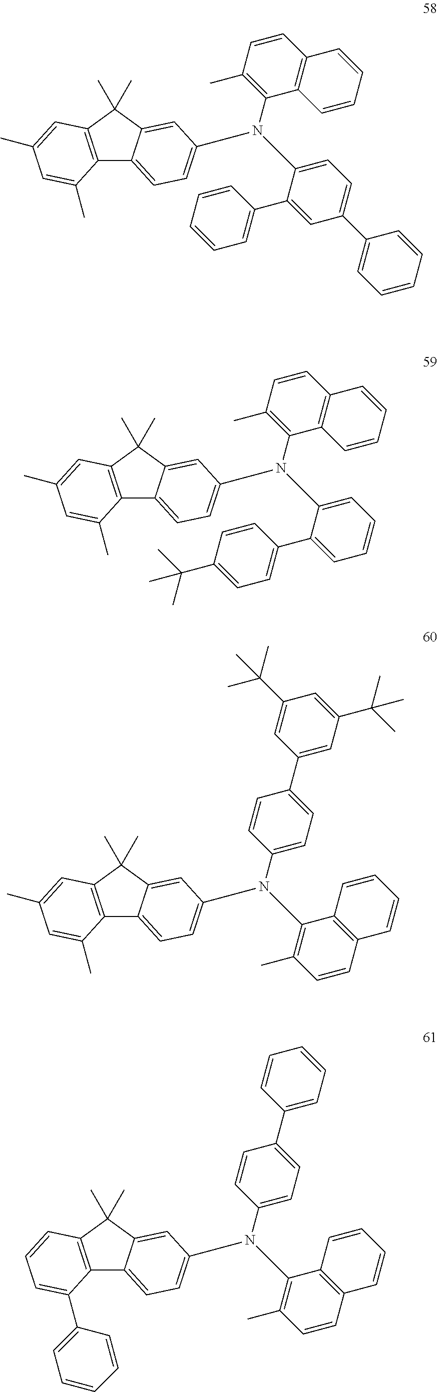 [SMILES: Cc1cc(C)c2c(c1)C(C)(C)c1cc(N(c3ccc(-c4ccccc4)cc3-c3ccccc3)c3c(C)ccc4ccccc34)ccc1-2 ; Cc1cc(C)c2c(c1)C(C)(C)c1cc(N(c3ccccc3-c3ccc(C(C)(C)C)cc3)c3c(C)ccc4ccccc34)ccc1-2 ; Cc1cc(C)c2c(c1)C(C)(C)c1cc(N(c3ccc(-c4cc(C(C)(C)C)cc(C(C)(C)C)c4)cc3)c3c(C)ccc4ccccc34)ccc1-2 ; Cc1ccc2ccccc2c1N(c1ccc(-c2ccccc2)cc1)c1ccc2c(c1)C(C)(C)c1cccc(-c3ccccc3)c1-2]

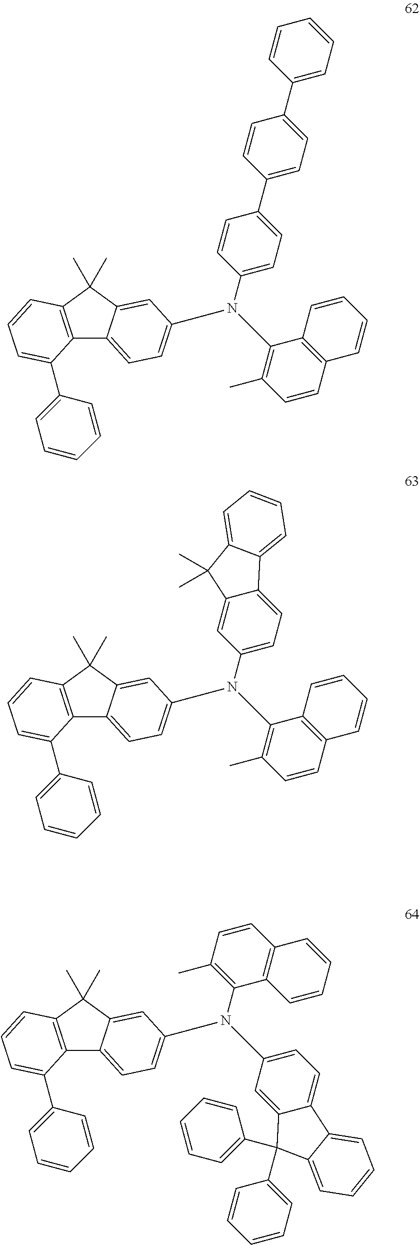 [SMILES: Cc1ccc2ccccc2c1N(c1ccc(-c2ccc(-c3ccccc3)cc2)cc1)c1ccc2c(c1)C(C)(C)c1cccc(-c3ccccc3)c1-2 ; Cc1ccc2ccccc2c1N(c1ccc2c(c1)C(C)(C)c1ccccc1-2)c1ccc2c(c1)C(C)(C)c1cccc(-c3ccccc3)c1-2 ; Cc1ccc2ccccc2c1N(c1ccc2c(c1)C(C)(C)c1cccc(-c3ccccc3)c1-2)c1ccc2c(c1)C(c1ccccc1)(c1ccccc1)c1ccccc1-2]

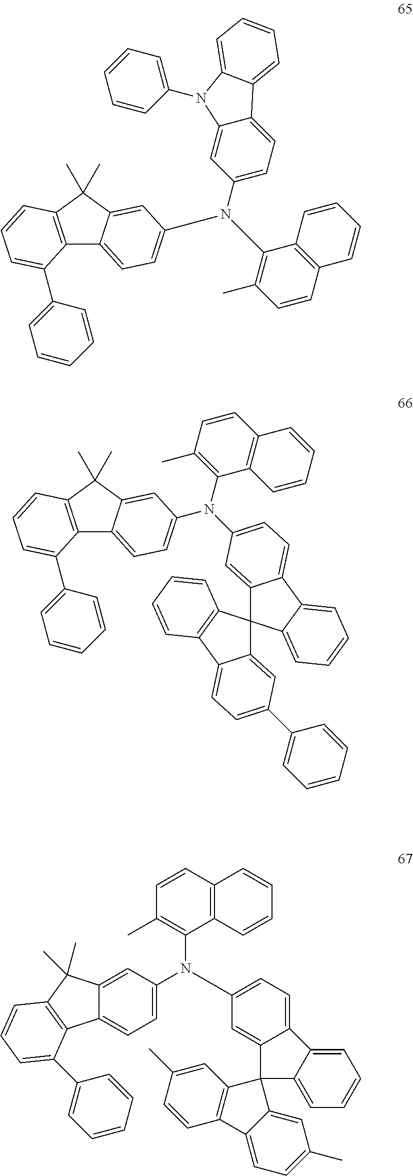 [SMILES: Cc1ccc2ccccc2c1N(c1ccc2c(c1)C(C)(C)c1cccc(-c3ccccc3)c1-2)c1ccc2c3ccccc3n(-c3ccccc3)c2c1 ; Cc1ccc2ccccc2c1N(c1ccc2c(c1)C(C)(C)c1cccc(-c3ccccc3)c1-2)c1ccc2c(c1)C1(c3ccccc3-c3ccc(-c4ccccc4)cc31)c1ccccc1-2 ; Cc1ccc2c(c1)C1(c3ccccc3-c3ccc(N(c4ccc5c(c4)C(C)(C)c4cccc(-c6ccccc6)c4-5)c4c(C)ccc5ccccc45)cc31)c1cc(C)ccc1-2]

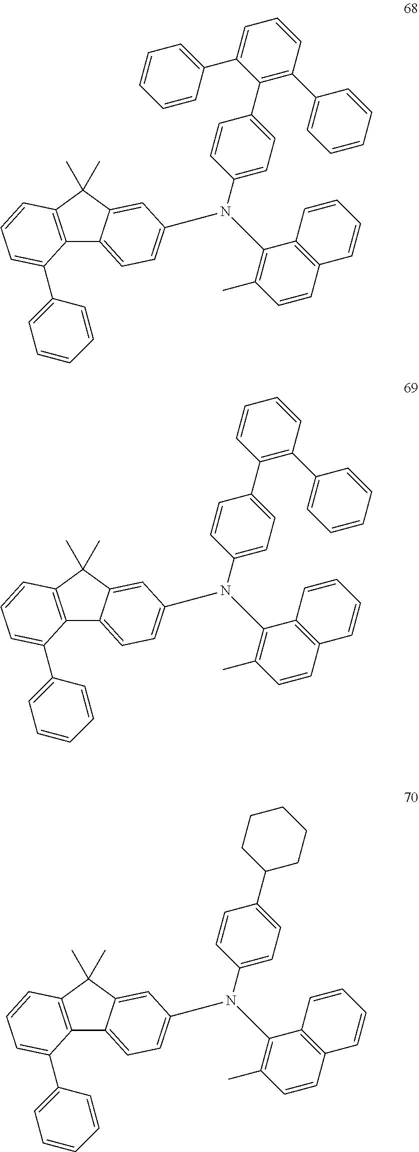 [SMILES: Cc1ccc2ccccc2c1N(c1ccc(-c2c(-c3ccccc3)cccc2-c2ccccc2)cc1)c1ccc2c(c1)C(C)(C)c1cccc(-c3ccccc3)c1-2 ; Cc1ccc2ccccc2c1N(c1ccc(-c2ccccc2-c2ccccc2)cc1)c1ccc2c(c1)C(C)(C)c1cccc(-c3ccccc3)c1-2 ; Cc1ccc2ccccc2c1N(c1ccc(C2CCCCC2)cc1)c1ccc2c(c1)C(C)(C)c1cccc(-c3ccccc3)c1-2]

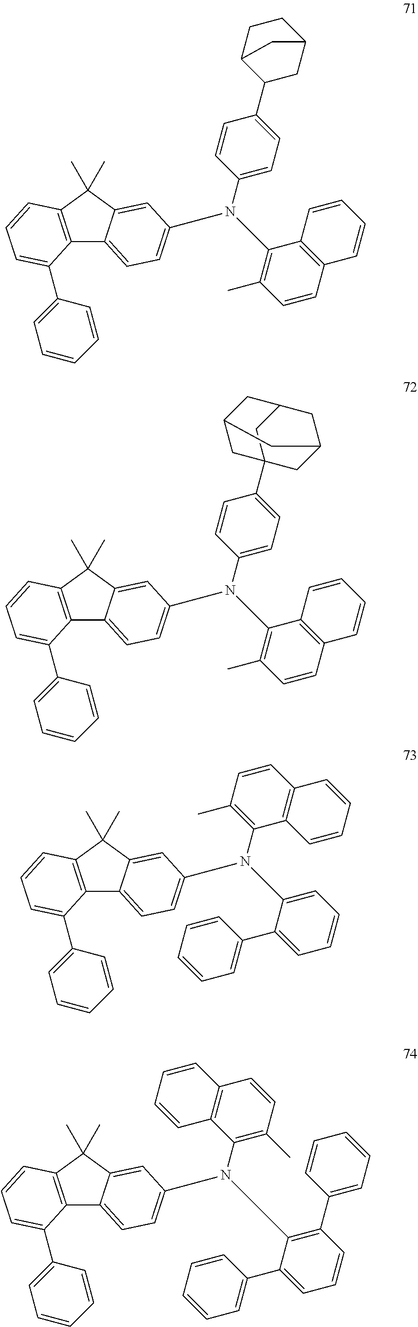 [SMILES: Cc1ccc2ccccc2c1N(c1ccc(C2CC3CCC2C3)cc1)c1ccc2c(c1)C(C)(C)c1cccc(-c3ccccc3)c1-2 ; Cc1ccc2ccccc2c1N(c1ccc(C23CC4CC(CC(C4)C2)C3)cc1)c1ccc2c(c1)C(C)(C)c1cccc(-c3ccccc3)c1-2 ; Cc1ccc2ccccc2c1N(c1ccc2c(c1)C(C)(C)c1cccc(-c3ccccc3)c1-2)c1ccccc1-c1ccccc1 ; Cc1ccc2ccccc2c1N(c1ccc2c(c1)C(C)(C)c1cccc(-c3ccccc3)c1-2)c1c(-c2ccccc2)cccc1-c1ccccc1]

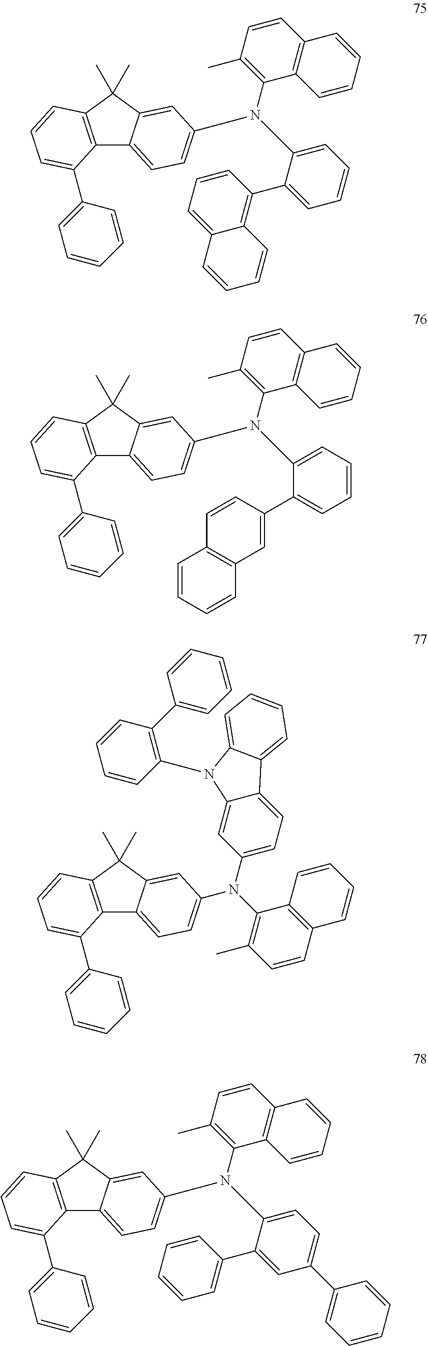

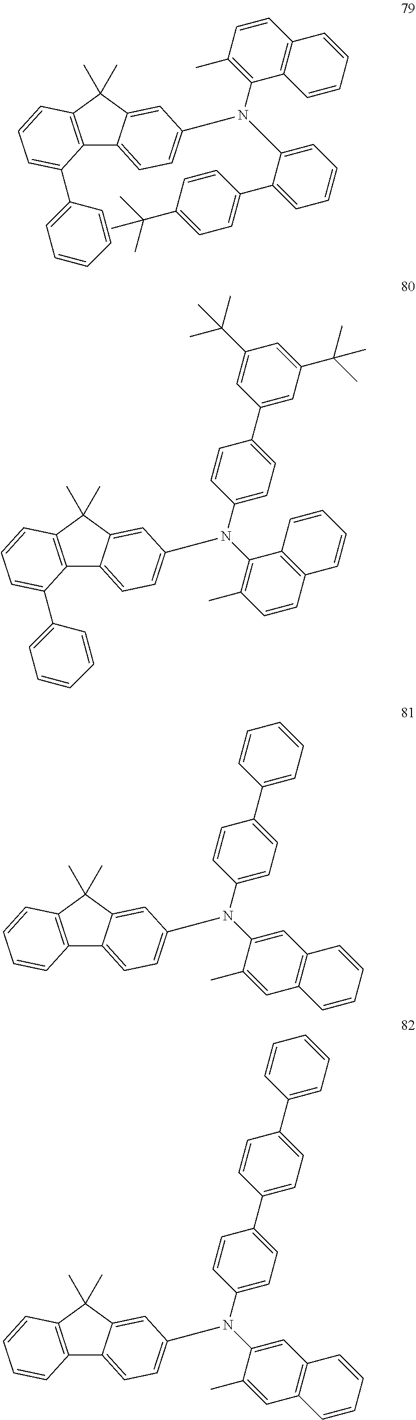

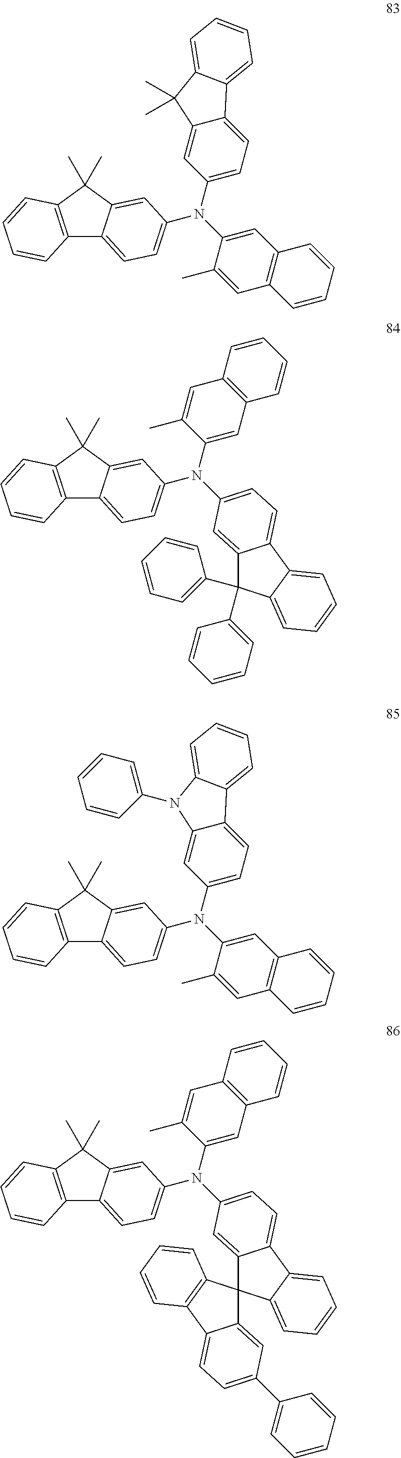

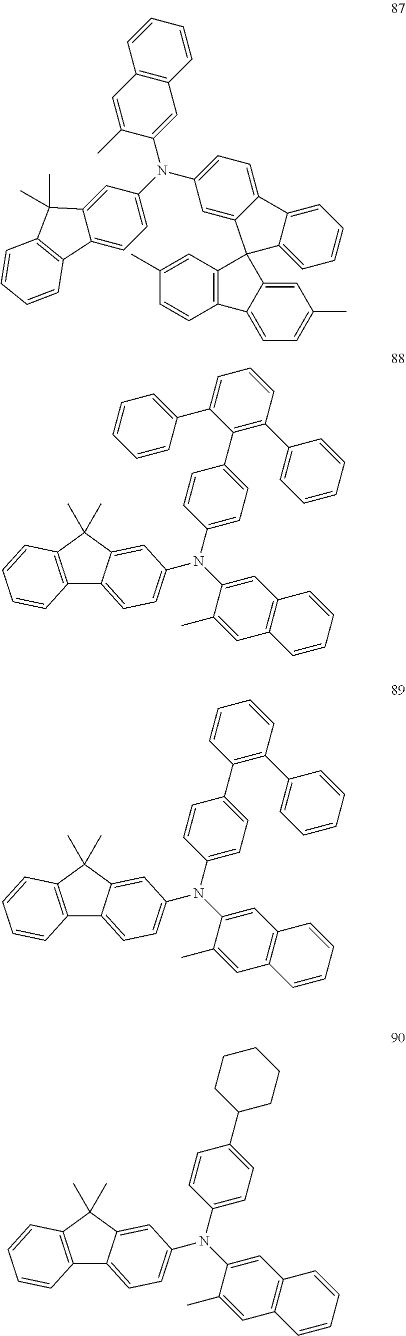

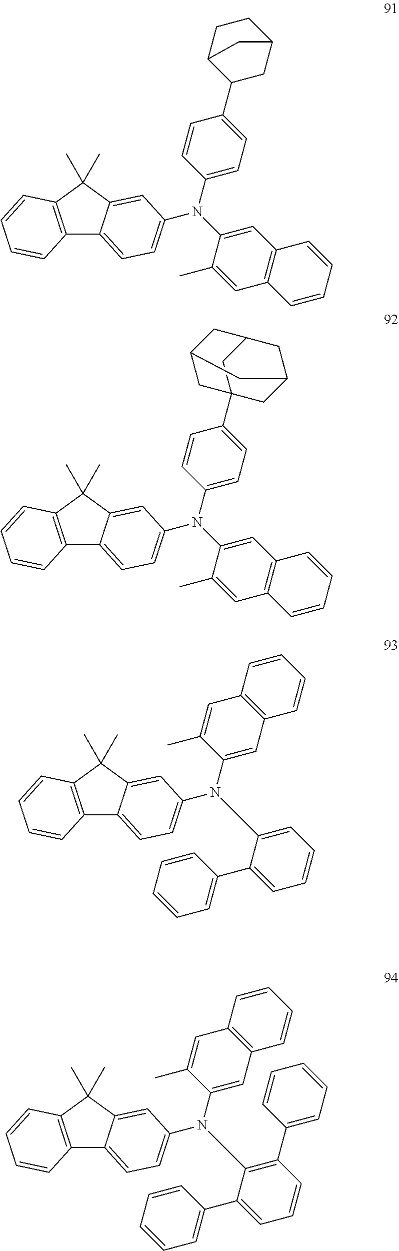

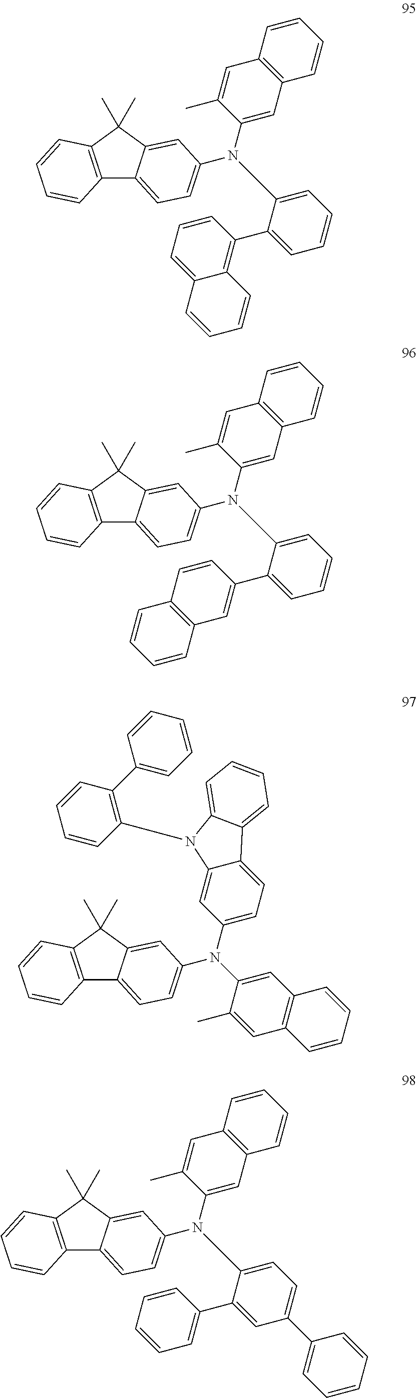

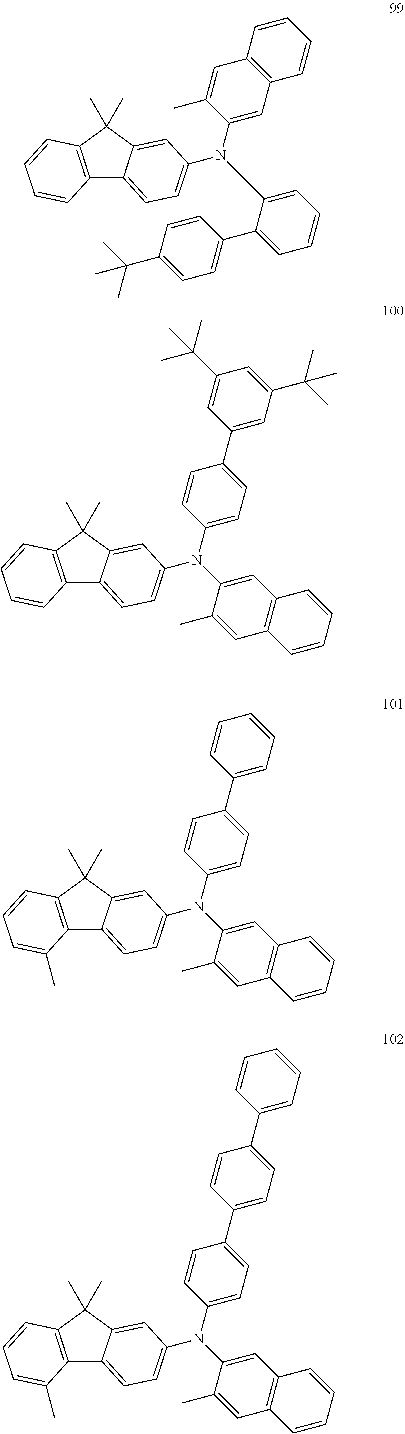

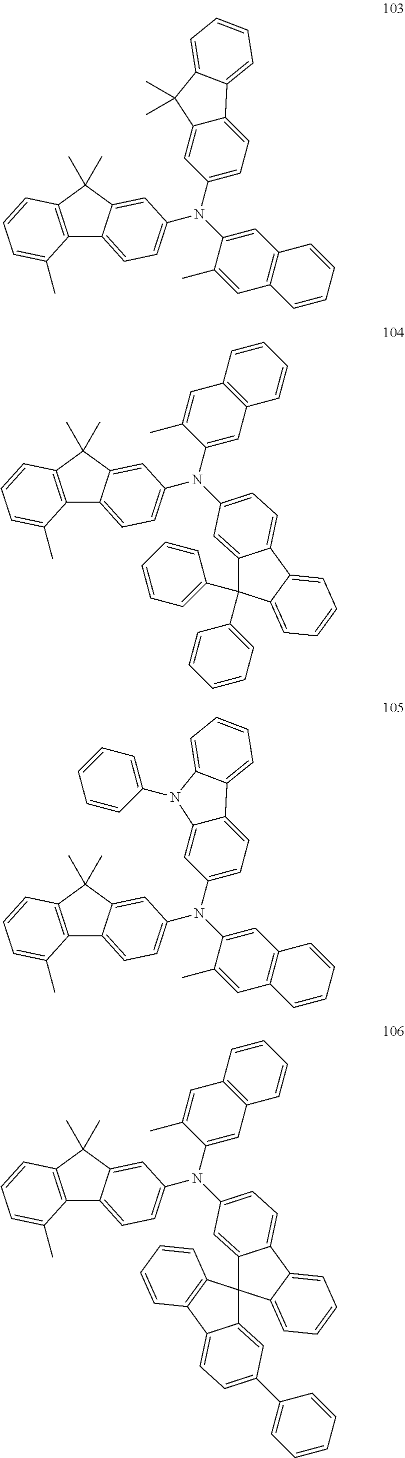

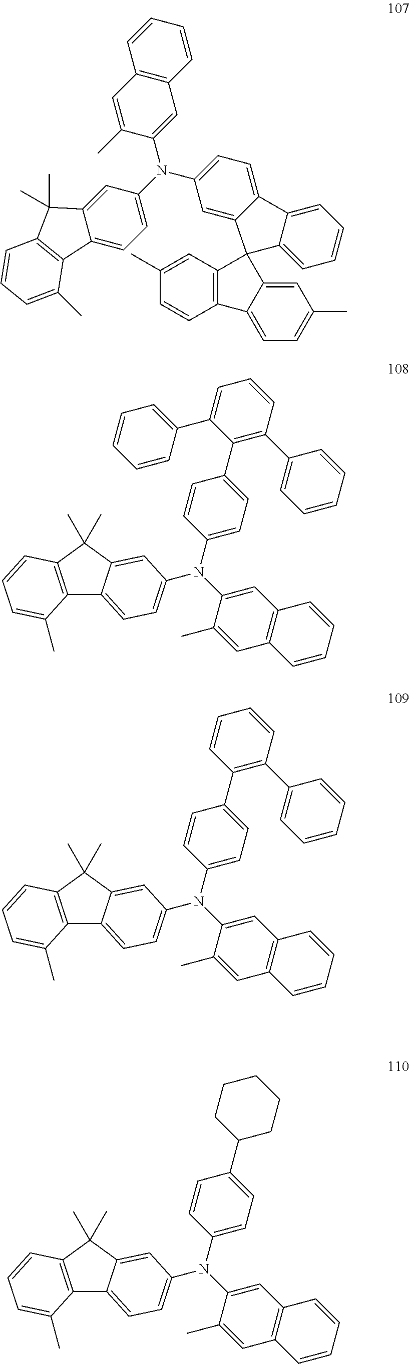

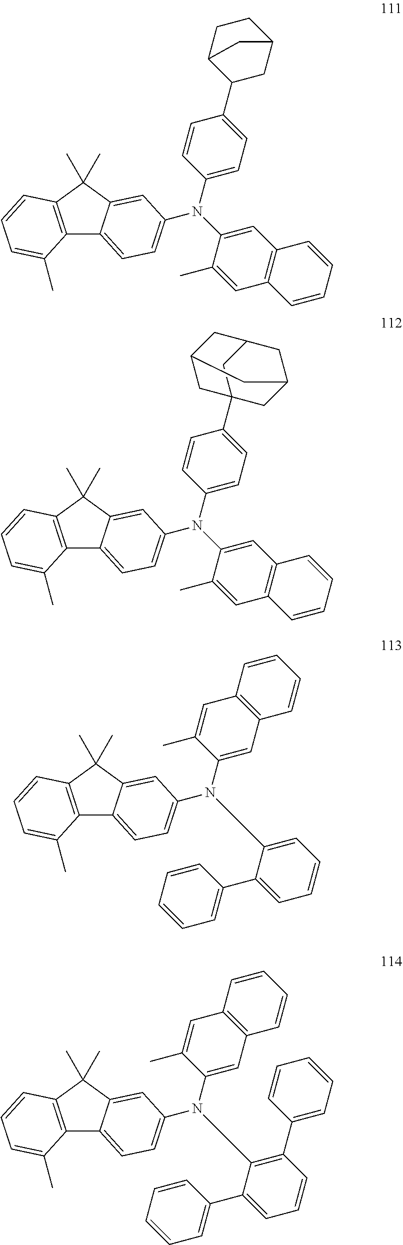

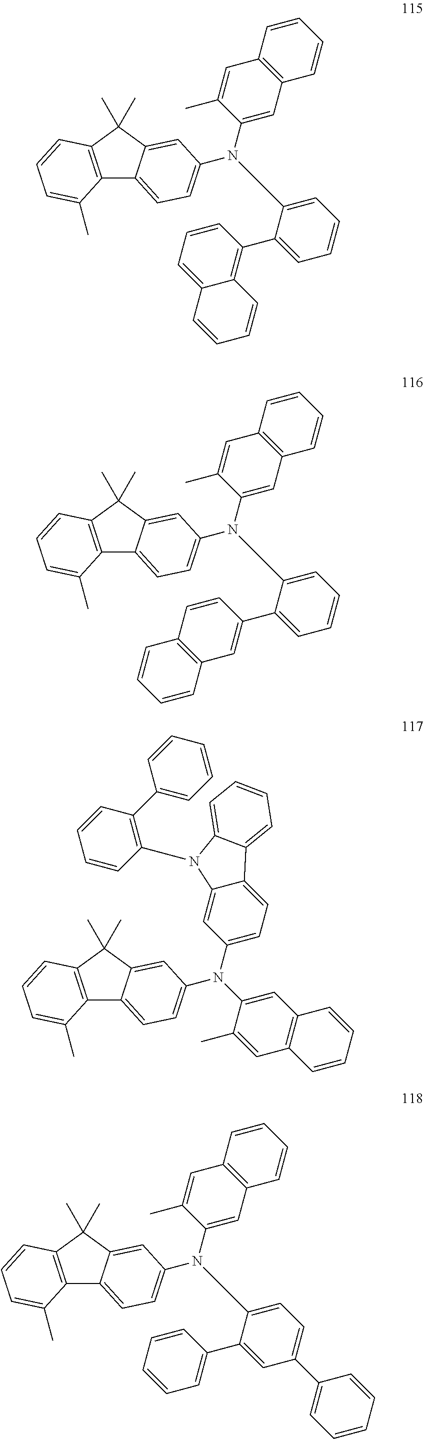

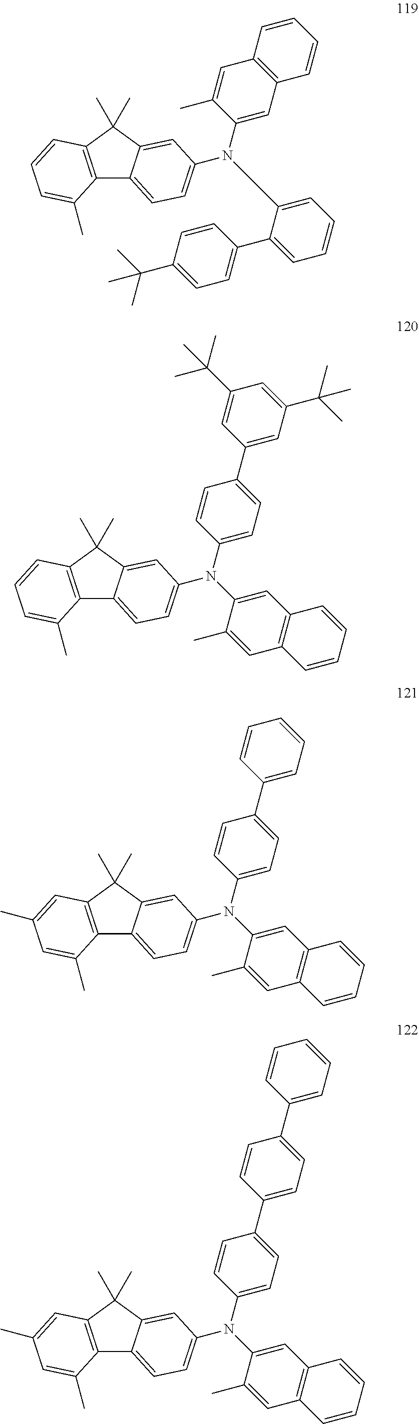

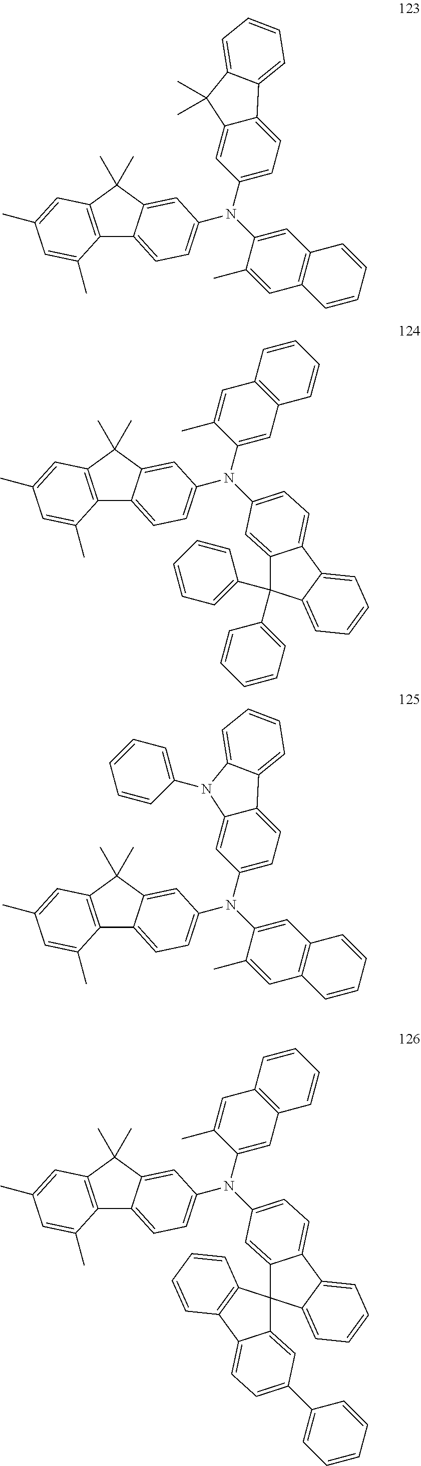

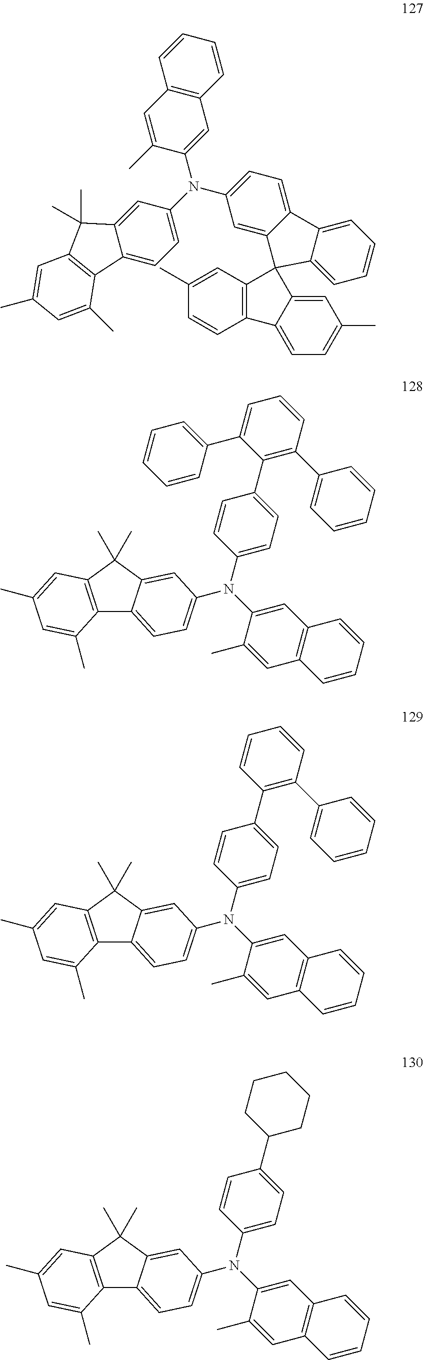

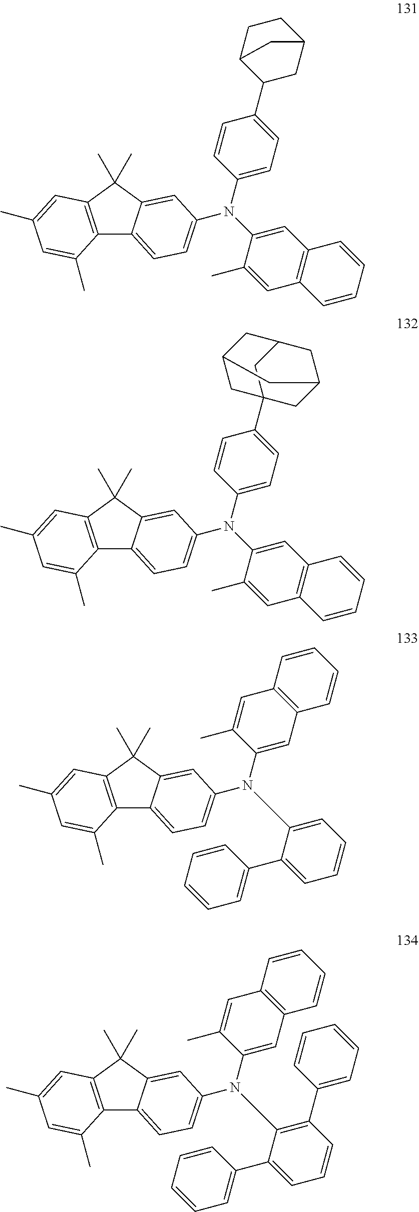

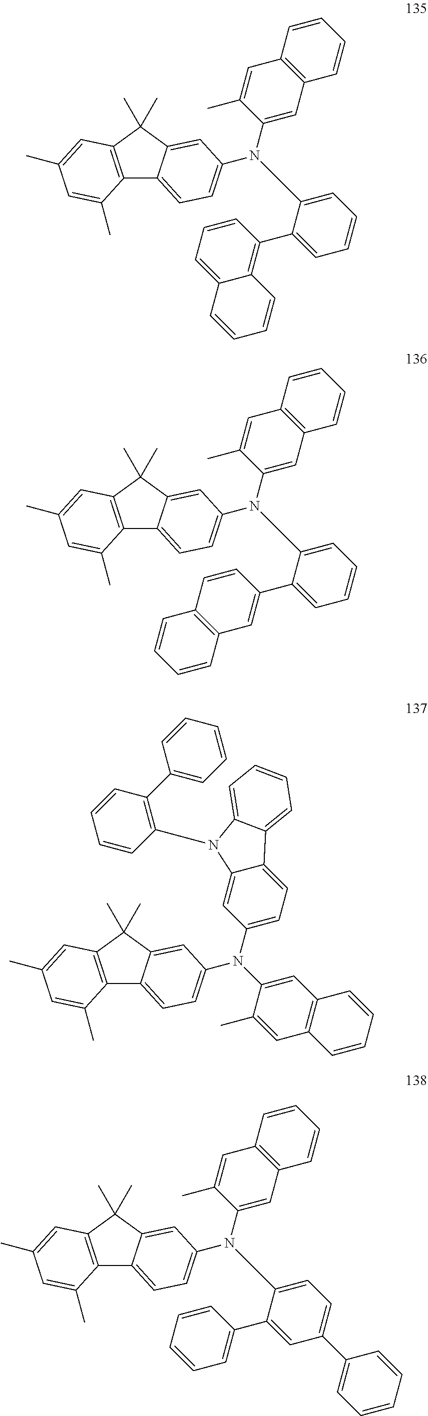

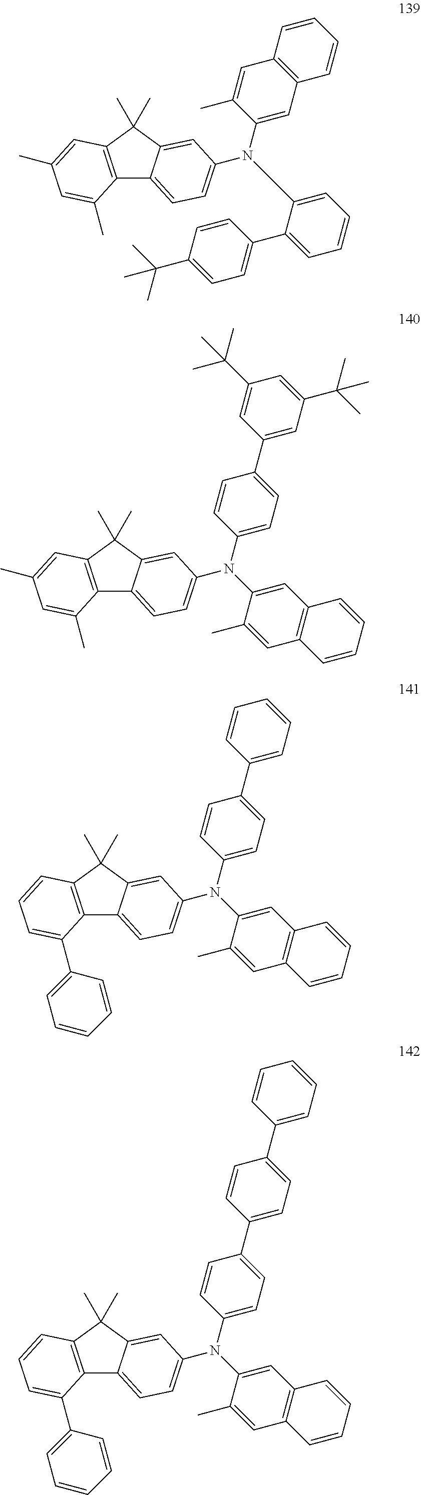 [SMILES: Cc1cc(C)c2c(c1)C(C)(C)c1cc(N(c3cc4ccccc4cc3C)c3ccccc3-c3ccc(C(C)(C)C)cc3)ccc1-2 ; Cc1cc(C)c2c(c1)C(C)(C)c1cc(N(c3ccc(-c4cc(C(C)(C)C)cc(C(C)(C)C)c4)cc3)c3cc4ccccc4cc3C)ccc1-2 ; Cc1cc2ccccc2cc1N(c1ccc(-c2ccccc2)cc1)c1ccc2c(c1)C(C)(C)c1cccc(-c3ccccc3)c1-2 ; Cc1cc2ccccc2cc1N(c1ccc(-c2ccc(-c3ccccc3)cc2)cc1)c1ccc2c(c1)C(C)(C)c1cccc(-c3ccccc3)c1-2]

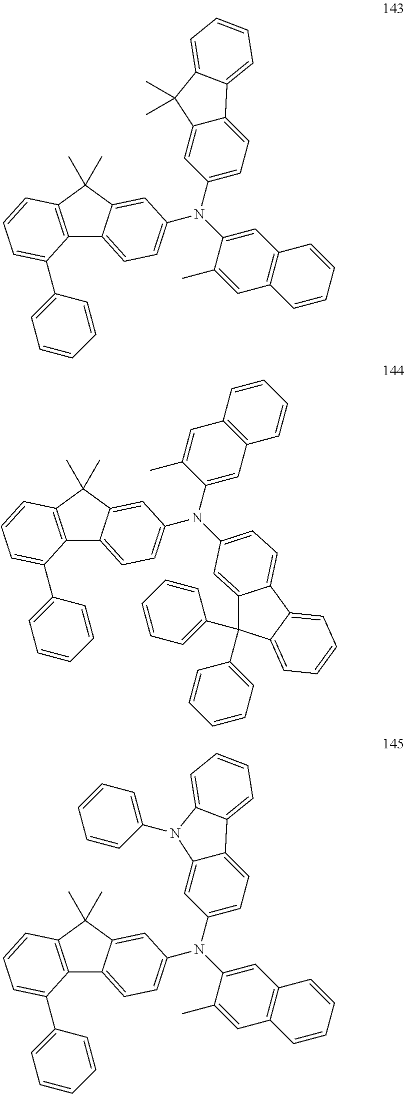

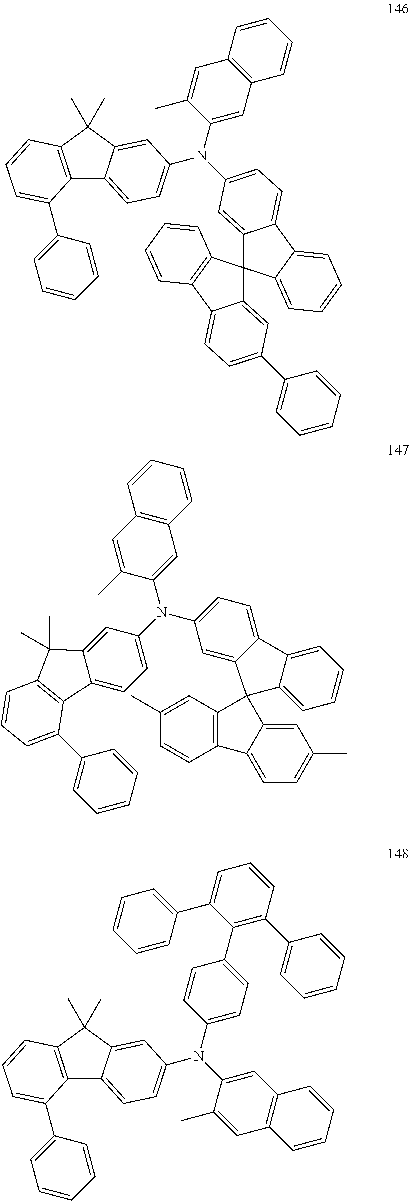 [SMILES: Cc1cc2ccccc2cc1N(c1ccc2c(c1)C(C)(C)c1cccc(-c3ccccc3)c1-2)c1ccc2c(c1)C1(c3ccccc3-c3ccc(-c4ccccc4)cc31)c1ccccc1-2 ; Cc1ccc2c(c1)C1(c3ccccc3-c3ccc(N(c4ccc5c(c4)C(C)(C)c4cccc(-c6ccccc6)c4-5)c4cc5ccccc5cc4C)cc31)c1cc(C)ccc1-2 ; Cc1cc2ccccc2cc1N(c1ccc(-c2c(-c3ccccc3)cccc2-c2ccccc2)cc1)c1ccc2c(c1)C(C)(C)c1cccc(-c3ccccc3)c1-2]

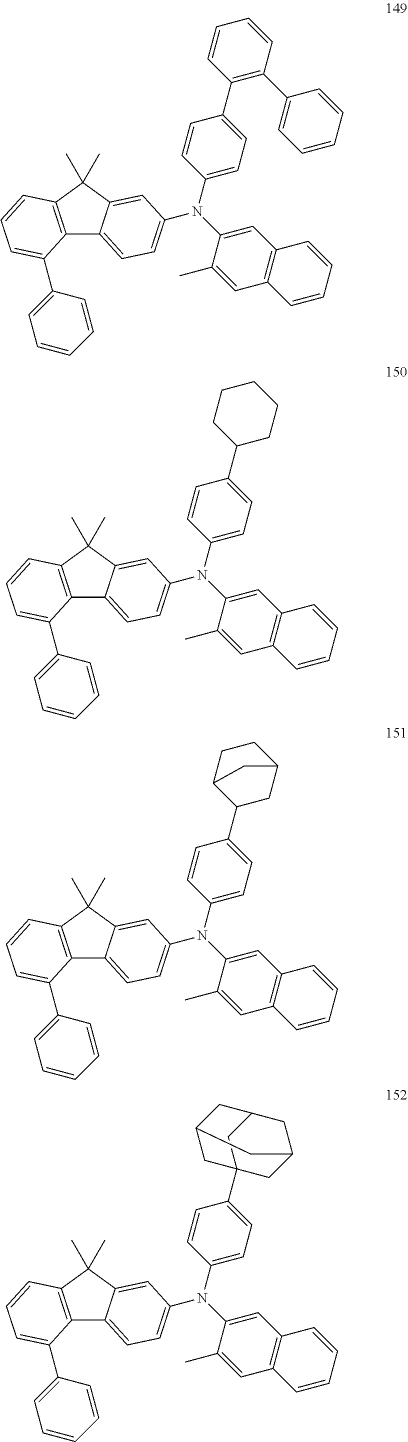 [SMILES: Cc1cc2ccccc2cc1N(c1ccc(-c2ccccc2-c2ccccc2)cc1)c1ccc2c(c1)C(C)(C)c1cccc(-c3ccccc3)c1-2 ; Cc1cc2ccccc2cc1N(c1ccc(C2CCCCC2)cc1)c1ccc2c(c1)C(C)(C)c1cccc(-c3ccccc3)c1-2 ; Cc1cc2ccccc2cc1N(c1ccc(C2CC3CCC2C3)cc1)c1ccc2c(c1)C(C)(C)c1cccc(-c3ccccc3)c1-2 ; Cc1cc2ccccc2cc1N(c1ccc(C23CC4CC(CC(C4)C2)C3)cc1)c1ccc2c(c1)C(C)(C)c1cccc(-c3ccccc3)c1-2]

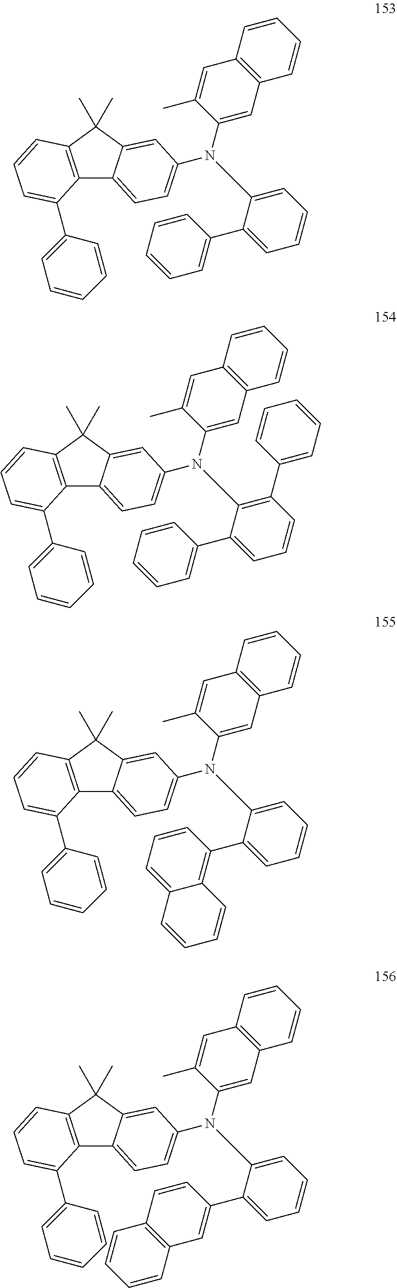 [SMILES: Cc1cc2ccccc2cc1N(c1ccc2c(c1)C(C)(C)c1cccc(-c3ccccc3)c1-2)c1ccccc1-c1ccccc1 ; Cc1cc2ccccc2cc1N(c1ccc2c(c1)C(C)(C)c1cccc(-c3ccccc3)c1-2)c1c(-c2ccccc2)cccc1-c1ccccc1 ; Cc1cc2ccccc2cc1N(c1ccc2c(c1)C(C)(C)c1cccc(-c3ccccc3)c1-2)c1ccccc1-c1cccc2ccccc12 ; Cc1cc2ccccc2cc1N(c1ccc2c(c1)C(C)(C)c1cccc(-c3ccccc3)c1-2)c1ccccc1-c1ccc2ccccc2c1]

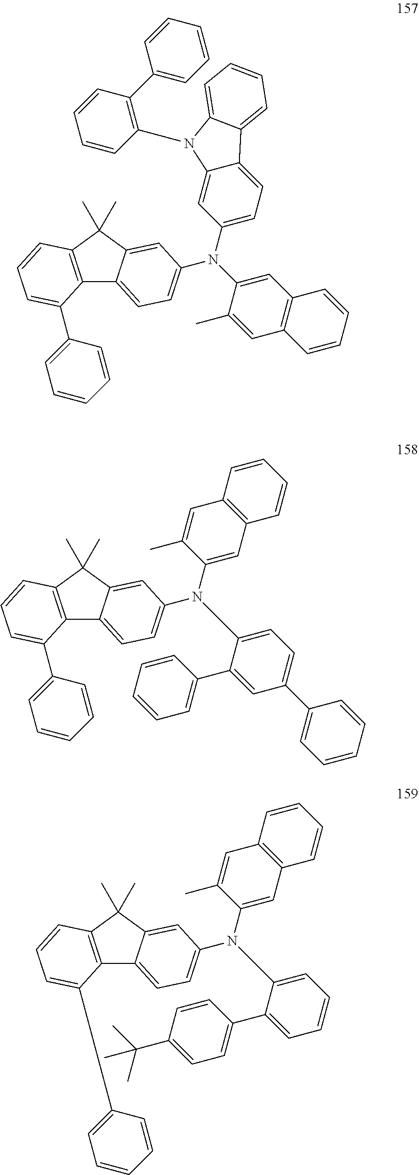

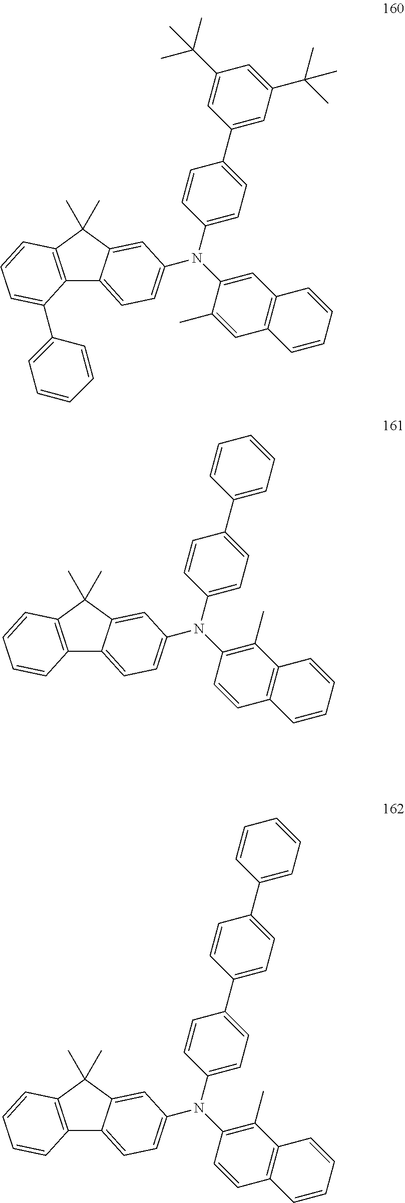 [SMILES: Cc1cc2ccccc2cc1N(c1ccc(-c2cc(C(C)(C)C)cc(C(C)(C)C)c2)cc1)c1ccc2c(c1)C(C)(C)c1cccc(-c3ccccc3)c1-2 ; Cc1c(N(c2ccc(-c3ccccc3)cc2)c2ccc3c(c2)C(C)(C)c2ccccc2-3)ccc2ccccc12 ; Cc1c(N(c2ccc(-c3ccc(-c4ccccc4)cc3)cc2)c2ccc3c(c2)C(C)(C)c2ccccc2-3)ccc2ccccc12]

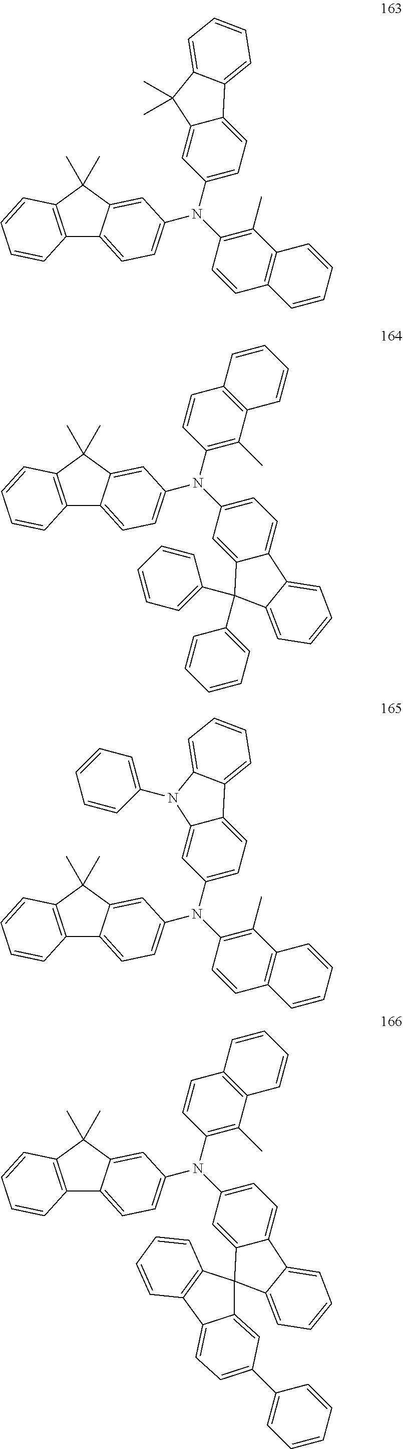

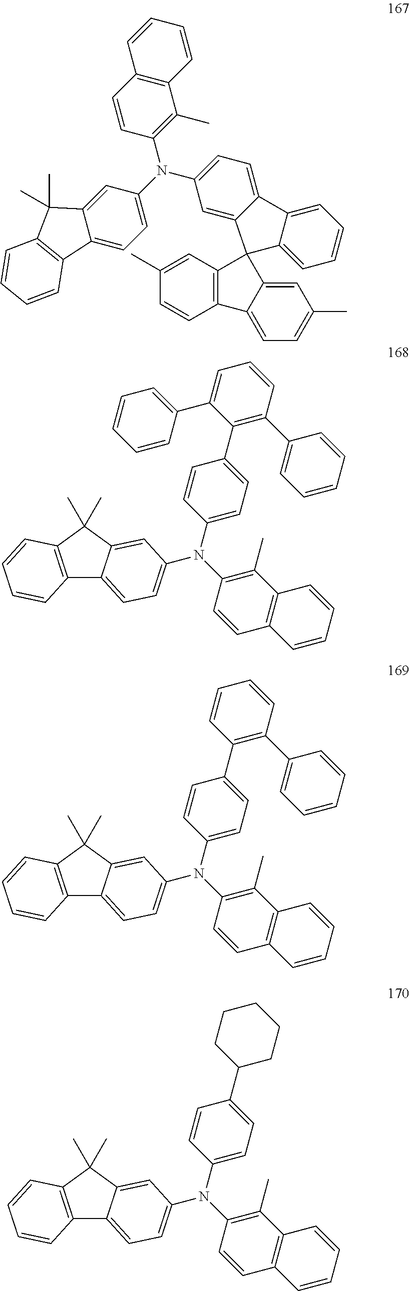

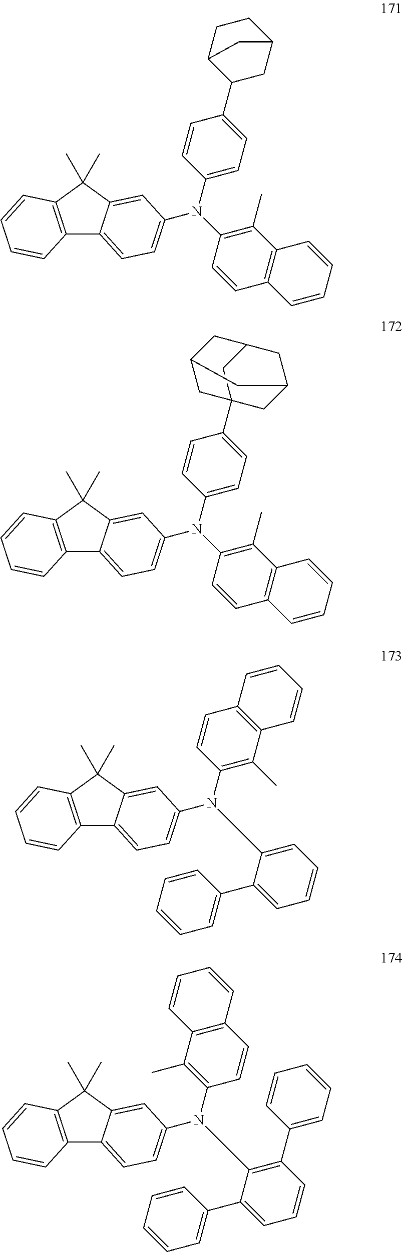

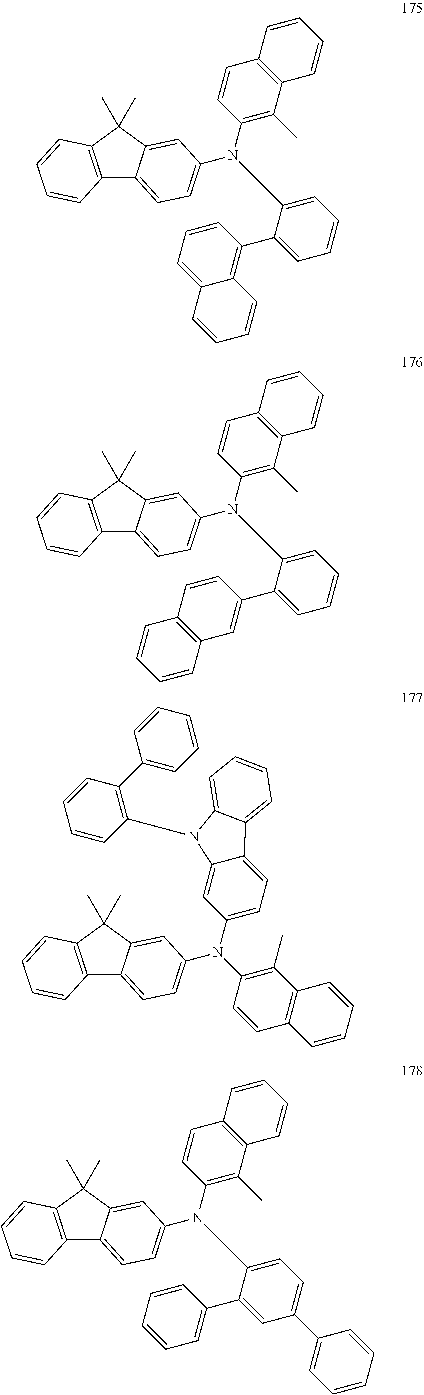

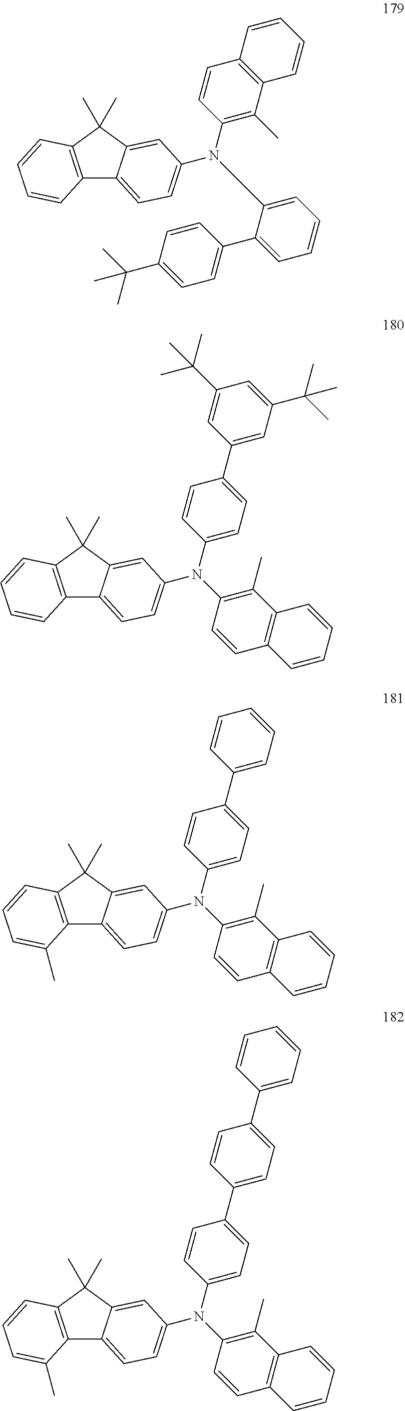

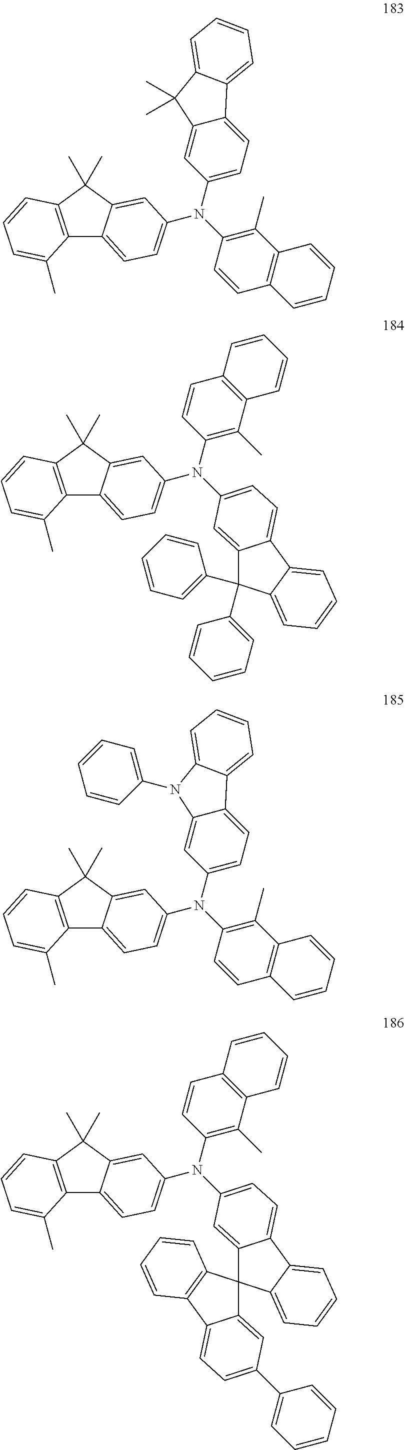

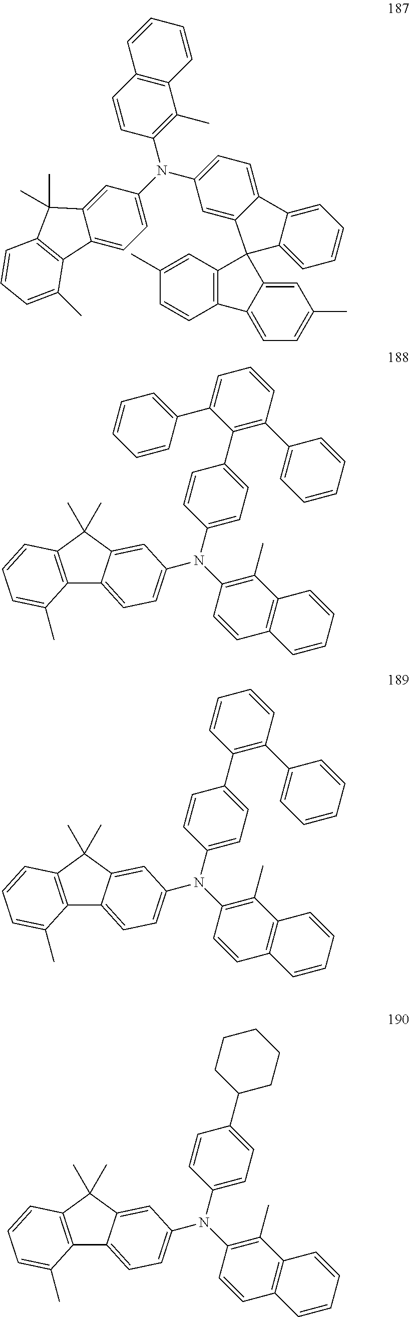

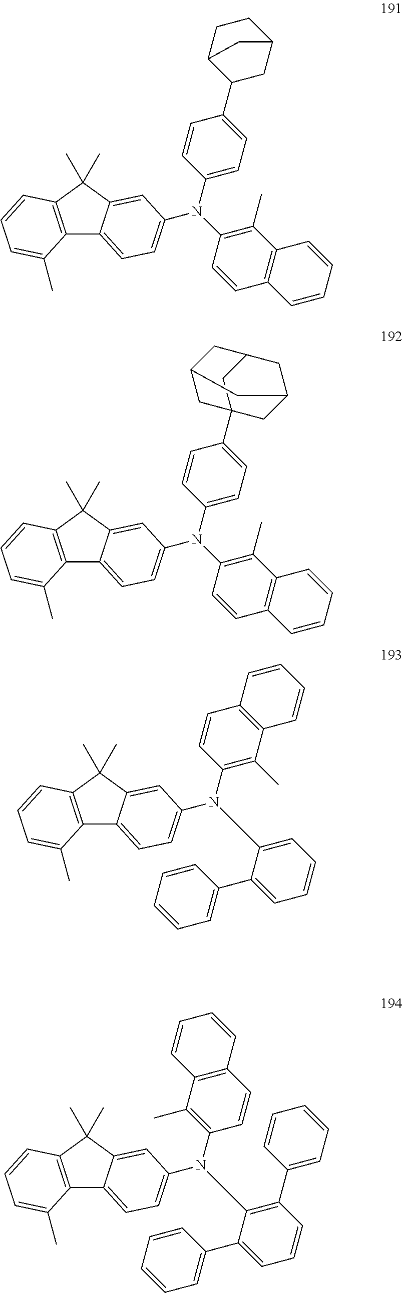

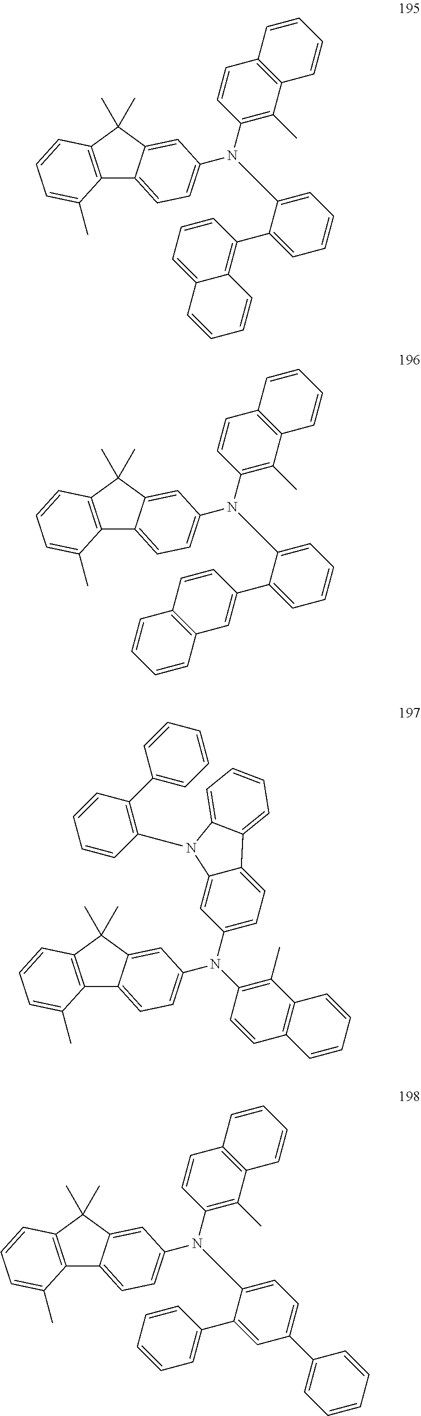 [SMILES: Cc1cccc2c1-c1ccc(N(c3ccccc3-c3cccc4ccccc34)c3ccc4ccccc4c3C)cc1C2(C)C ; Cc1cccc2c1-c1ccc(N(c3ccccc3-c3ccc4ccccc4c3)c3ccc4ccccc4c3C)cc1C2(C)C ; Cc1cccc2c1-c1ccc(N(c3ccc4c5ccccc5n(-c5ccccc5-c5ccccc5)c4c3)c3ccc4ccccc4c3C)cc1C2(C)C ; Cc1cccc2c1-c1ccc(N(c3ccc(-c4ccccc4)cc3-c3ccccc3)c3ccc4ccccc4c3C)cc1C2(C)C]

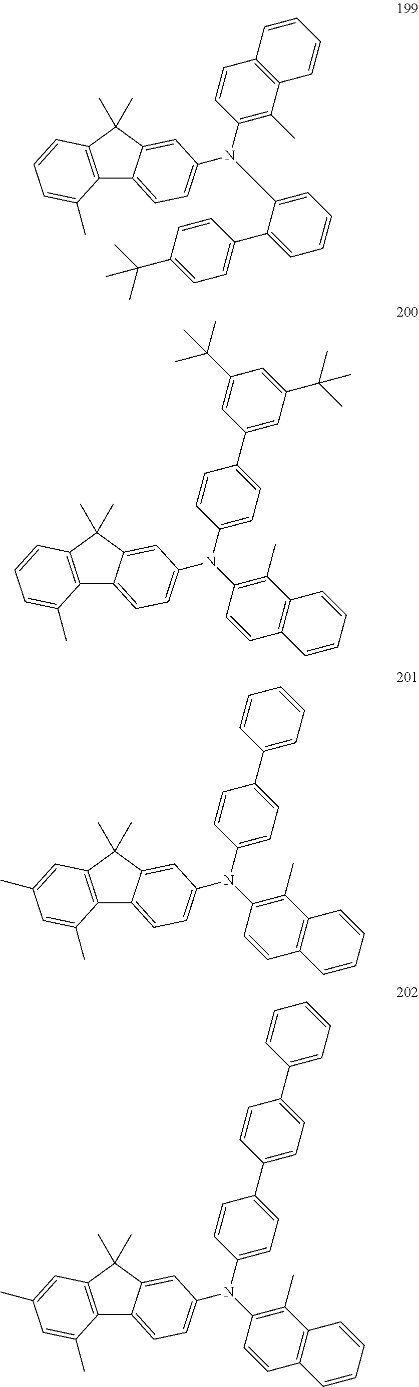

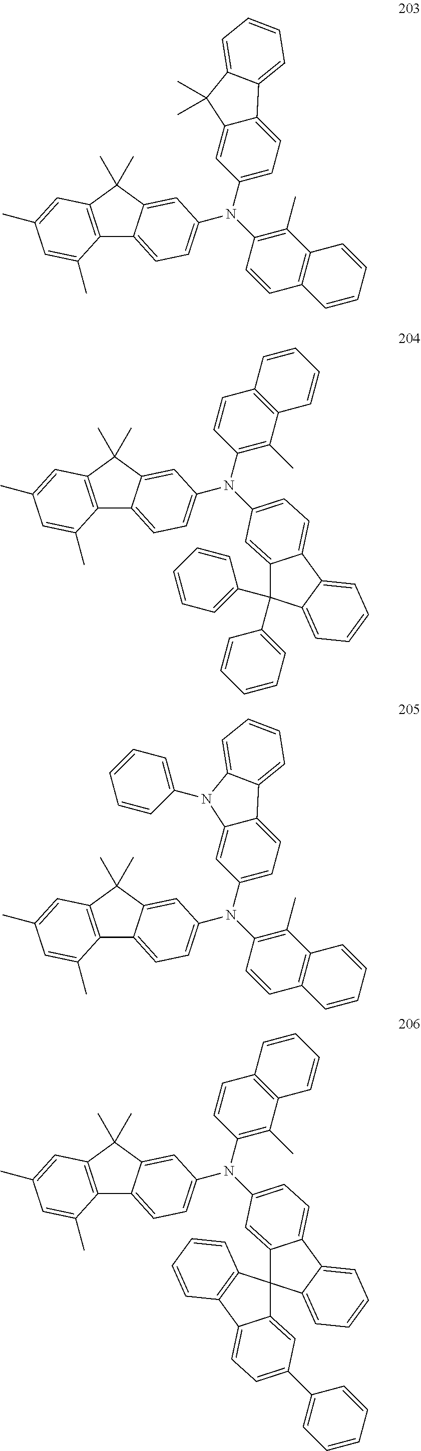

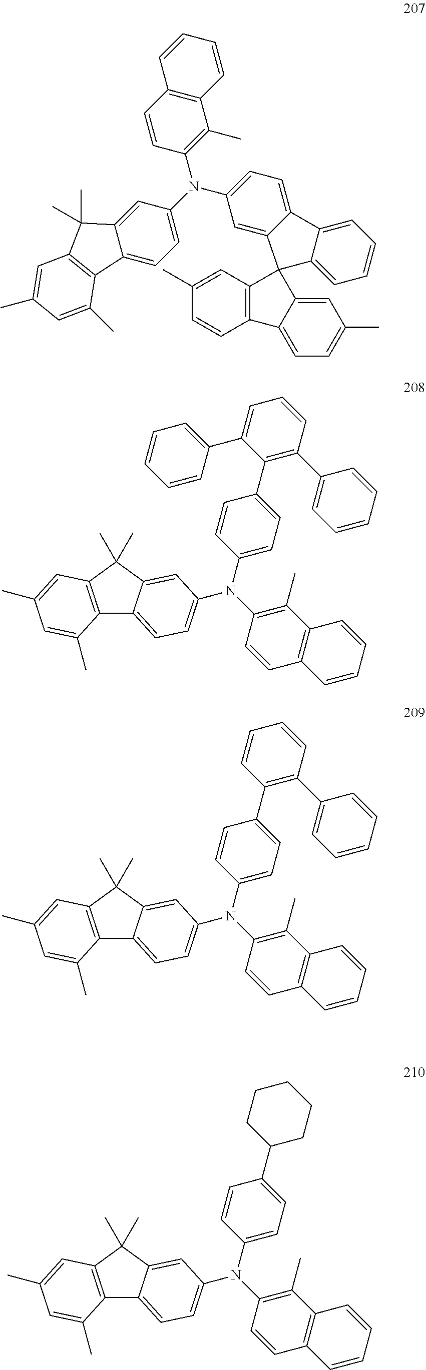

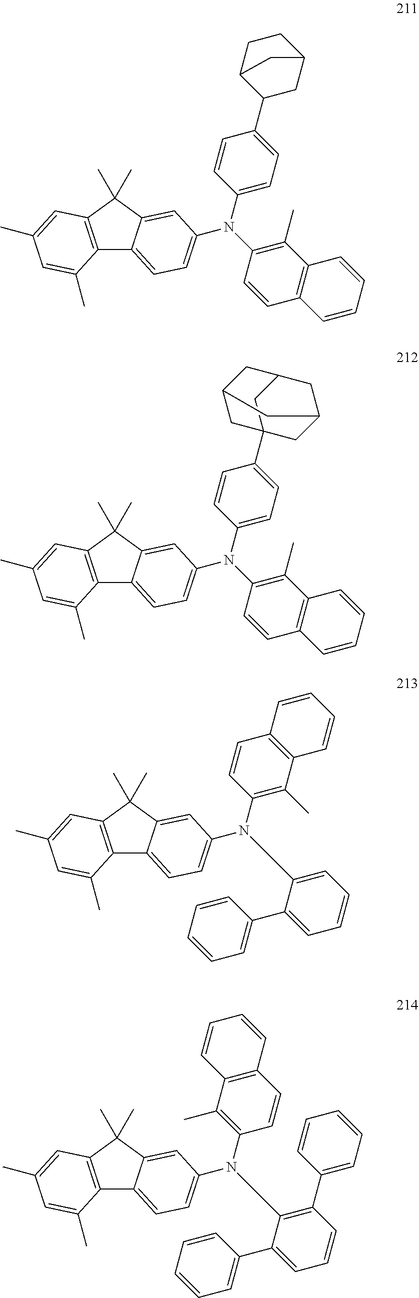

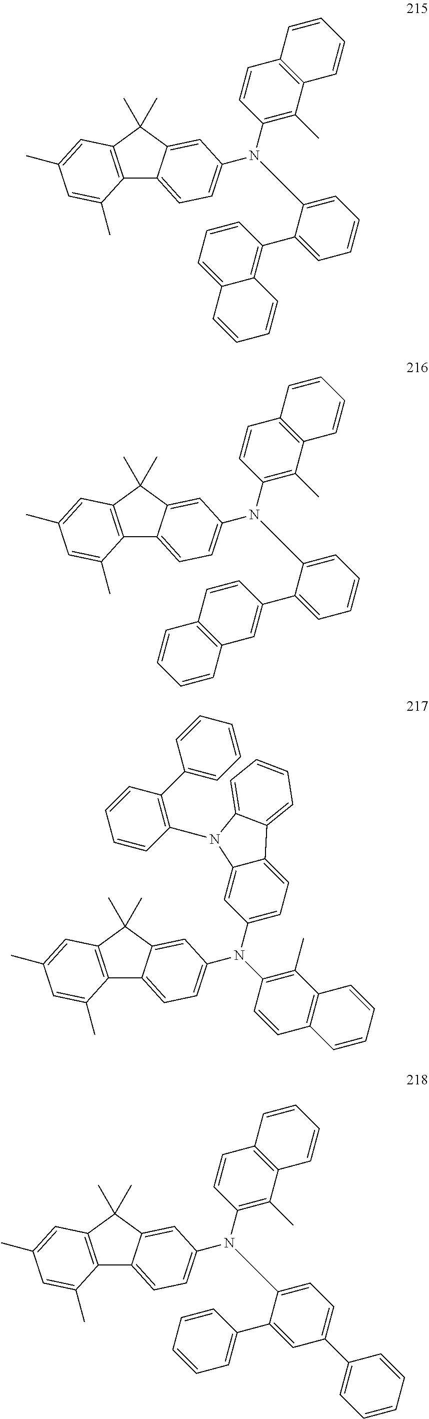

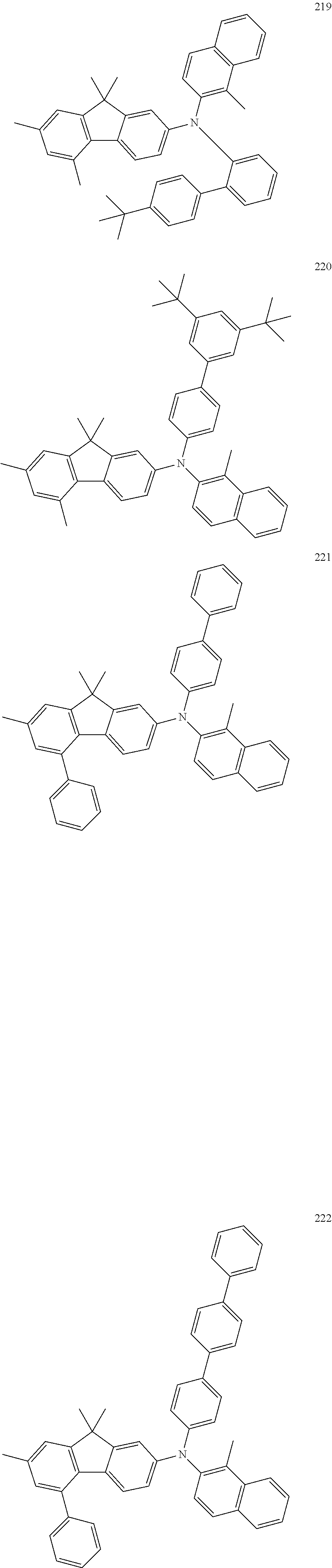

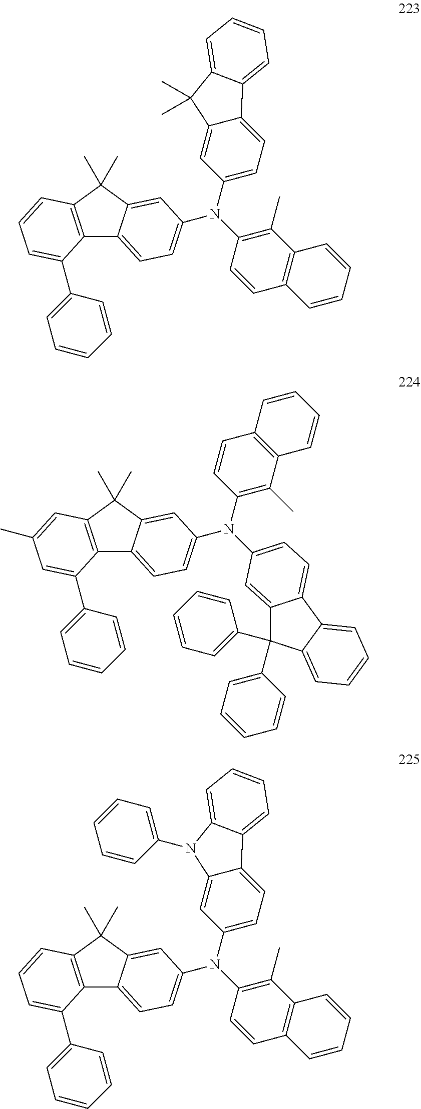

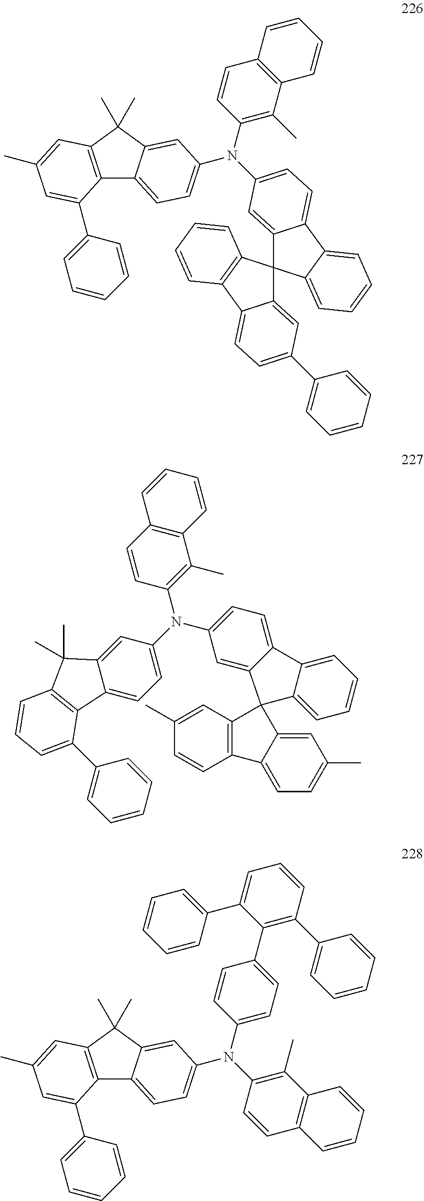

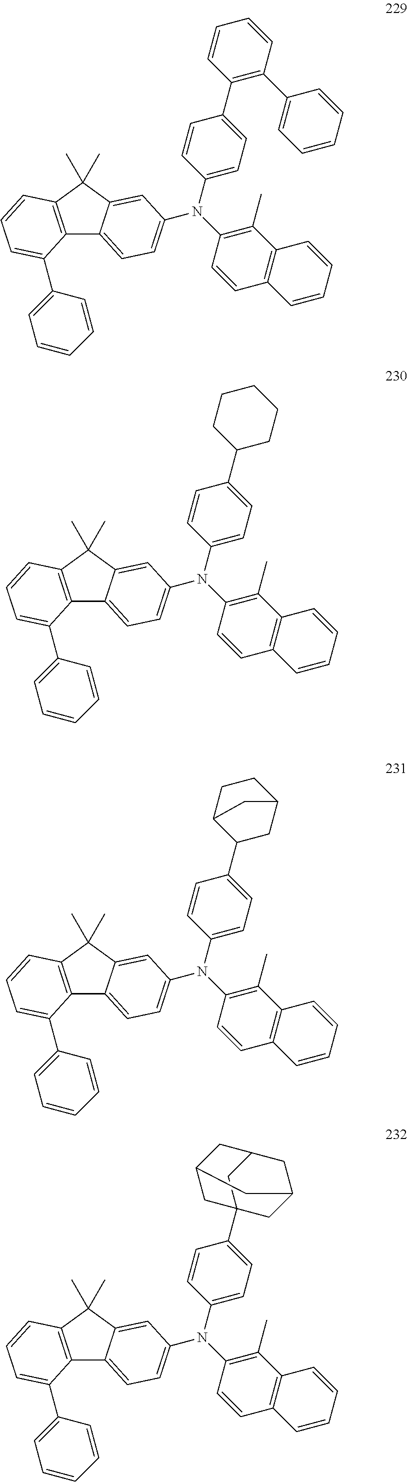 [SMILES: Cc1c(N(c2ccc(-c3ccccc3-c3ccccc3)cc2)c2ccc3c(c2)C(C)(C)c2cccc(-c4ccccc4)c2-3)ccc2ccccc12 ; Cc1c(N(c2ccc(C3CCCCC3)cc2)c2ccc3c(c2)C(C)(C)c2cccc(-c4ccccc4)c2-3)ccc2ccccc12 ; Cc1c(N(c2ccc(C3CC4CCC3C4)cc2)c2ccc3c(c2)C(C)(C)c2cccc(-c4ccccc4)c2-3)ccc2ccccc12 ; Cc1c(N(c2ccc(C34CC5CC(CC(C5)C3)C4)cc2)c2ccc3c(c2)C(C)(C)c2cccc(-c4ccccc4)c2-3)ccc2ccccc12]

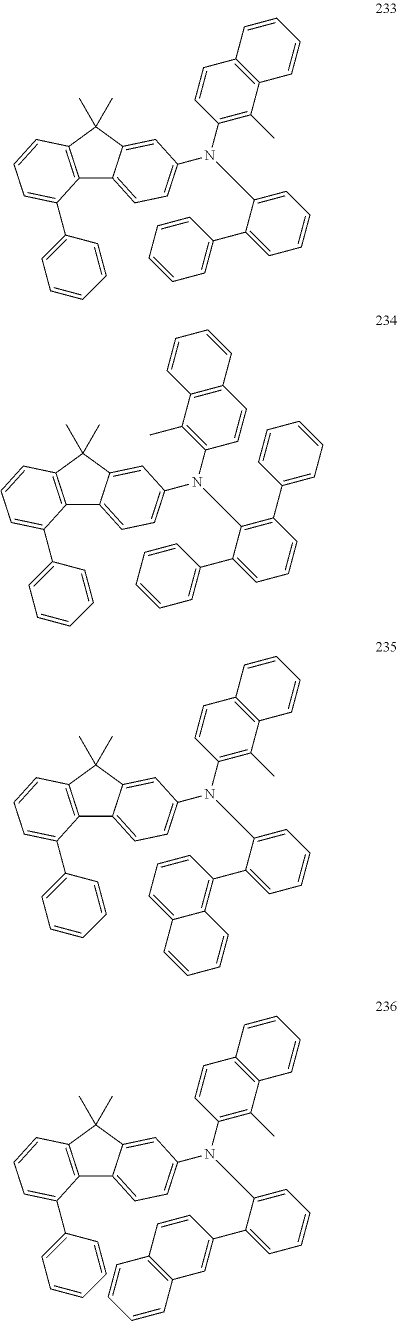 [SMILES: Cc1c(N(c2ccc3c(c2)C(C)(C)c2cccc(-c4ccccc4)c2-3)c2ccccc2-c2ccccc2)ccc2ccccc12 ; Cc1c(N(c2ccc3c(c2)C(C)(C)c2cccc(-c4ccccc4)c2-3)c2c(-c3ccccc3)cccc2-c2ccccc2)ccc2ccccc12 ; Cc1c(N(c2ccc3c(c2)C(C)(C)c2cccc(-c4ccccc4)c2-3)c2ccccc2-c2cccc3ccccc23)ccc2ccccc12 ; Cc1c(N(c2ccc3c(c2)C(C)(C)c2cccc(-c4ccccc4)c2-3)c2ccccc2-c2ccc3ccccc3c2)ccc2ccccc12]

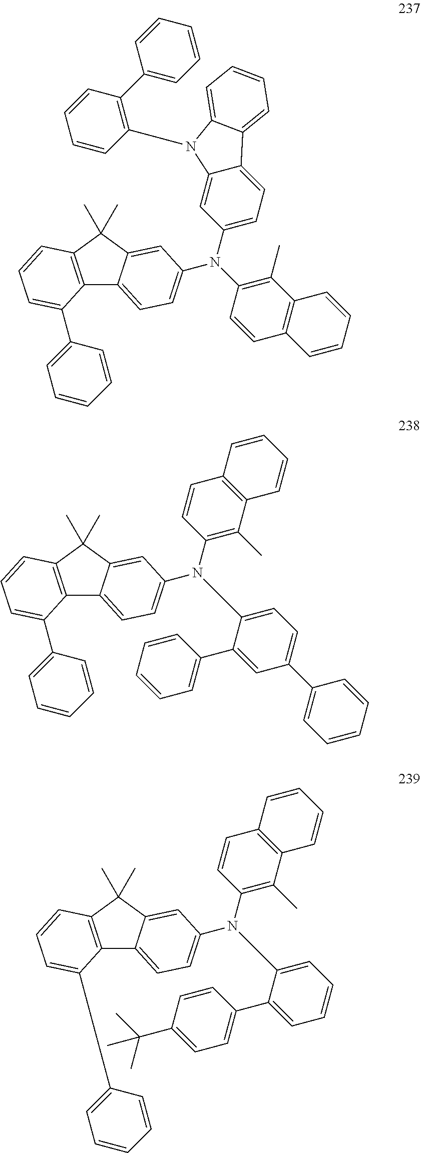 [SMILES: Cc1c(N(c2ccc3c(c2)C(C)(C)c2cccc(-c4ccccc4)c2-3)c2ccc3c4ccccc4n(-c4ccccc4-c4ccccc4)c3c2)ccc2ccccc12 ; Cc1c(N(c2ccc3c(c2)C(C)(C)c2cccc(-c4ccccc4)c2-3)c2ccc(-c3ccccc3)cc2-c2ccccc2)ccc2ccccc12 ; Cc1c(N(c2ccc3c(c2)C(C)(C)c2cccc(-c4ccccc4)c2-3)c2ccccc2-c2ccc(C(C)(C)C)cc2)ccc2ccccc12]

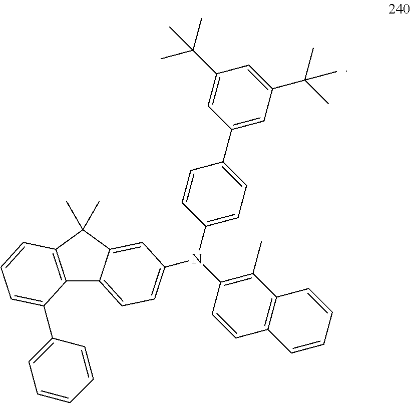 [SMILES: Cc1c(N(c2ccc(-c3cc(C(C)(C)C)cc(C(C)(C)C)c3)cc2)c2ccc3c(c2)C(C)(C)c2cccc(-c4ccccc4)c2-3)ccc2ccccc12]

The amine compound according to an embodiment includes a first substituent, a second substituent and a third substituent, and a light emitting element including the amine compound may have a high efficiency, a low voltage, a high luminance, and a long lifetime.

The amine compound of an embodiment includes an amine group and has a structure in which the first to third substituents are combined with the amine group of the amine compound of an embodiment. In this case, the first substituent is a naphthalene group at which at least one alkyl group is essentially substituted. If the carbon atom at position 1 of the naphthalene moiety of the first substituent is connected with the amine group, the carbon atom at position 2 may be connected with the alkyl group, and if the carbon atom at position 2 of the naphthalene moiety is connected with the amine group, the carbon atom at position 1 or the carbon atom at position 3 may be connected with the alkyl group. The second substituent may include a fluorenyl moiety, and the third substituent may be an alkyl group, an alkenyl group, an alkynyl group, an aryl group or a heteroaryl group.

The amine compound of an embodiment, having such a structure essentially includes the first substituent and the second substituent, and the highest occupied molecular orbital (HOMO) energy level of a molecule may be adjusted. The hole injection barrier between the first electrode EL1 and the hole transport region HTR may be changed, and a suitable energy level may be achieved between the hole transport region HTR and the emission layer EML. Accordingly, the amine compound of an embodiment may be controlled to increase exciton production efficiency in the emission layer EML. In the amine compound of an embodiment, an alkyl group in the first substituent is bonded to the naphthalene moiety at the ortho position with respect to the core nitrogen atom, and the amine compound may have a high glass-transition temperature (Tg) properties to prevent or reduce the crystallization of the amine compound. Accordingly, the amine compound of an embodiment may have high thermal properties. Accordingly, if the amine compound according to embodiment of the present disclosure is applied to the hole transport region HTR of the light emitting element ED, a light emitting element having a high efficiency, a low voltage, a high luminance and a long lifetime may be accomplished.

If the amine compound of an embodiment is used in the hole transport region, a light extraction mode between the first electrode and the second electrode may be changed to increase external quantum efficiency. Accordingly, if the amine compound of an embodiment is used in the hole transport region, the emission efficiency of the light emitting element may be increased, and the lifetime of the light emitting element may be improved.

In the light emitting element ED of an embodiment, the hole transport region HTR may further include a compound represented by Formula H-1 below.

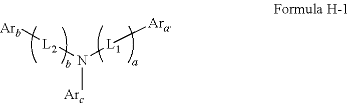

In Formula H-1 above, L₁ and L₂ may be each independently a direct linkage, a substituted or unsubstituted arylene group of 6 to 30 ring-forming carbon atoms, or a substituted or unsubstituted heteroarylene group of 2 to 30 ring-forming carbon atoms. “a” and “b” may be each independently an integer of 0 to 10. In some embodiments, if “a” or “b” is an integer of 2 or more, a plurality of L₁ and L₂ may be each independently a substituted or unsubstituted arylene group of 6 to 30 ring-forming carbon atoms, or a substituted or unsubstituted heteroarylene group of 2 to 30 ring-forming carbon atoms.

In Formula H-1, Arₐ and Ar_{b} may be each independently a substituted or unsubstituted aryl group of 6 to 30 ring-forming carbon atoms, or a substituted or unsubstituted heteroaryl group of 2 to 30 ring-forming carbon atoms. In addition, in Formula H-1, Ar_{c} may be a substituted or unsubstituted aryl group of 6 to 30 ring-forming carbon atoms.

The compound represented by Formula H-1 may be a monoamine compound. In some embodiments, the compound represented by Formula H-1 may be a diamine compound in which at least one selected from among Arₐ to Ar_{c} includes an amine group as a substituent. In addition, the compound represented by Formula H-1 may be a carbazole-based compound in which at least one selected from among Arₐ and Ar_{b} includes a substituted or unsubstituted carbazole group, or a fluorene-based compound in which at least one selected from among Arₐ and Ar_{b} includes a substituted or unsubstituted fluorene group.

The compound represented by Formula H-1 may be represented by any one selected from among the compounds in Compound Group H below. However, the compounds listed in Compound Group H are only illustrations, and the compound represented by Formula H-1 is not limited to the compounds represented in Compound Group H below.

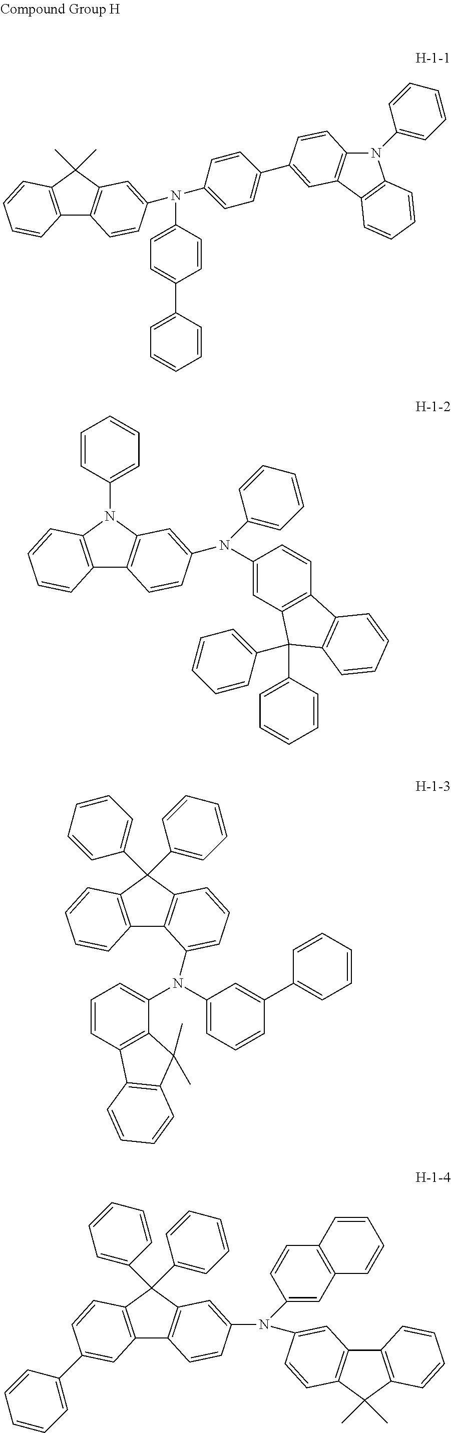 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21 ; CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21]

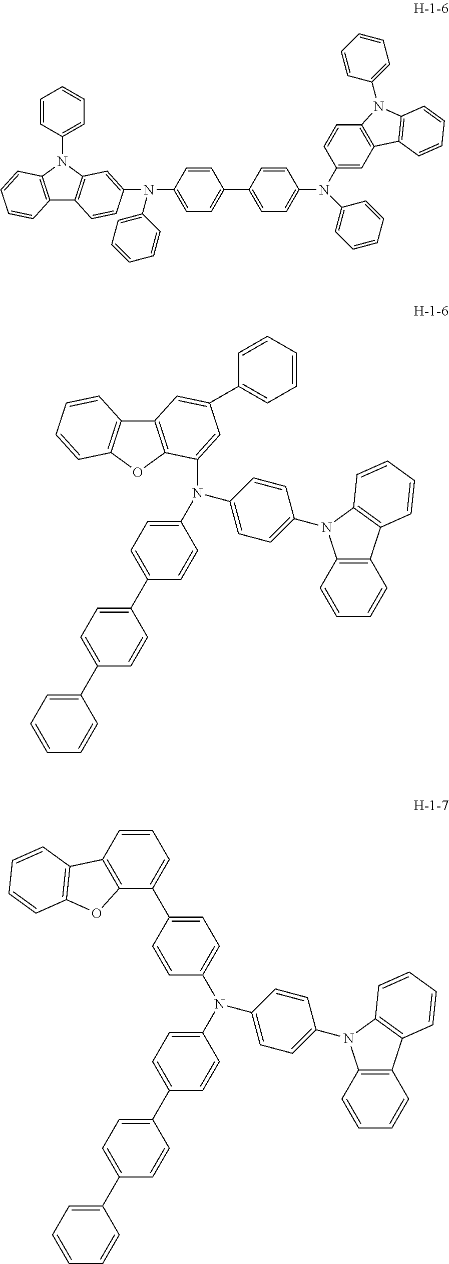 [SMILES: c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

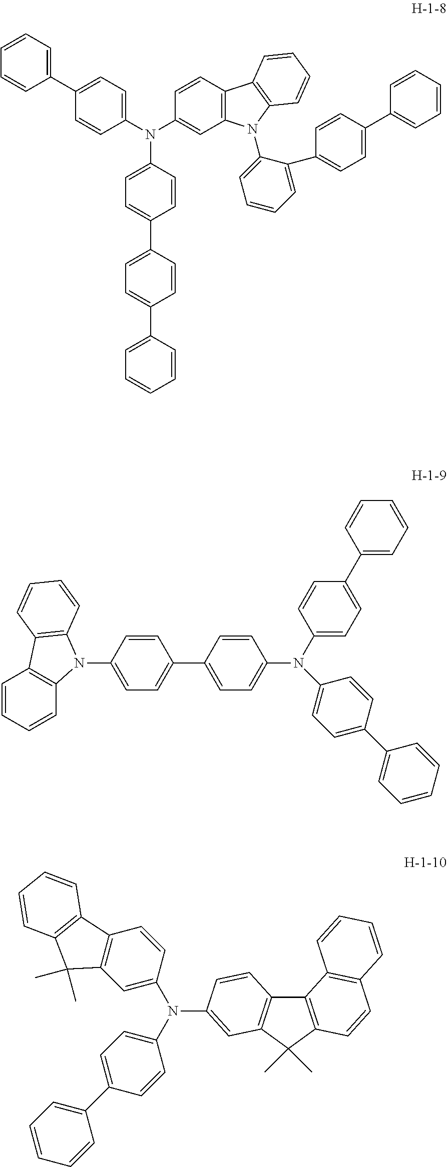 [SMILES: c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2ccccc2-c2ccc(N(c3ccc(-c4ccccc4)cc3)c3ccc4c(c3)C(C)(C)c3ccc5ccccc5c3-4)cc21]

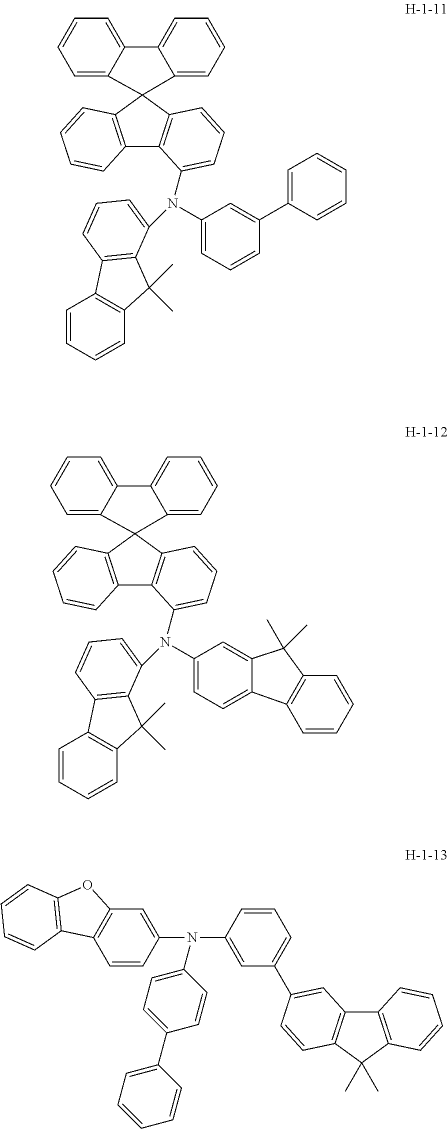 [SMILES: CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21]

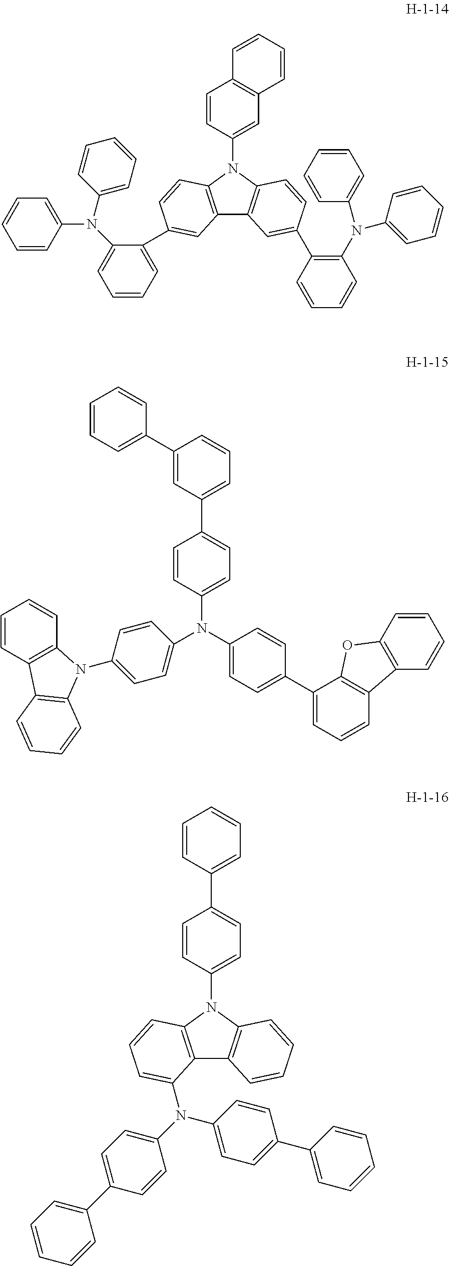 [SMILES: c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1]

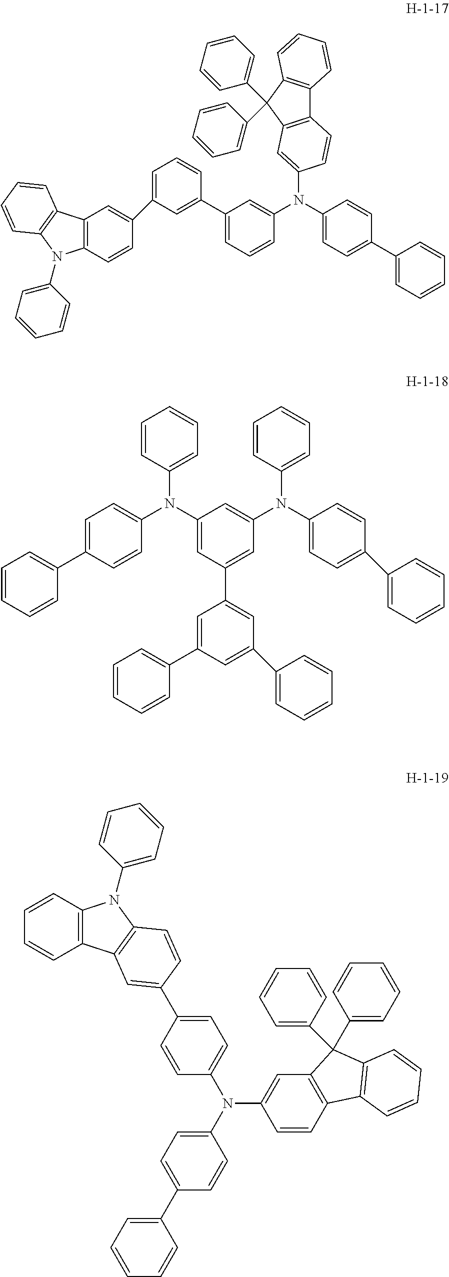 [SMILES: c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

The hole transport region HTR may further include any suitable hole transport material generally used in the art.

In some embodiments, the hole transport region HTR may include a phthalocyanine compound such as copper phthalocyanine, N¹,N¹′-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino] triphenylamine (m-MTDATA), 4,4′,4″-tris(N,N-diphenylamino)triphenylamine (TDATA), 4,4′,4″-tris[N(1-naphthyl)-N-phenylamino]-triphenylamine (1-TNATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(1-naphthalene-1-yl)-N,N′-diphenyl-benzidine (NPB or NPD), triphenylamine-containing polyetherketone (TPAPEK), 4-isopropyl-4′-methyldiphenyliodonium [tetrakis(pentafluorophenyl)borate], and/or dipyrazino[2,3-f:2′,3′-h] quinoxaline-2,3,6,7,10,11-hexacarbonitrile (HAT-CN).

The hole transport region HTR may include carbazole derivatives such as N-phenyl carbazole and/or polyvinyl carbazole, fluorene-based derivatives, triphenylamine-based derivatives such as 4,4′,4″-tris(N-carbazolyl)triphenylamine (TCTA), N,N′-bis(3-methylphenyl)-N,N′-diphenyl-[1,1′-biphenyl]-4,4′-diamine (TPD), N,N′-di(naphthalene-1-yl)-N,N′-diphenyl-benzidine (NPB), 4,4′-cyclohexylidene bis[N,N-bis(4-methylphenyl)benzeneamine] (TAPC), 4,4′-bis[N,N′-(3-tolyl)amino]-3,3′-dimethylbiphenyl (HMTPD), 1,3-bis(N-carbazolyl)benzene (mCP), etc.

In addition, the hole transport region HTR may include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), etc.

The hole transport region HTR may include the compounds of the hole transport region in at least one selected from among a hole injection layer HIL, hole transport layer HTL, and electron blocking layer EBL.

The thickness of the hole transport region HTR may be from about 100 Å to about 10,000 Å, for example, from about 100 Å to about 5,000 Å. If the hole transport region HTR includes a hole injection layer HIL, the thickness of the hole injection region HIL may be, for example, from about 30 Å to about 1,000 Å. If the hole transport region HTR includes a hole transport layer HTL, the thickness of the hole transport layer HTL may be from about 30 Å to about 1,000 Å. For example, if the hole transport region HTR includes an electron blocking layer EBL, the thickness of the electron blocking layer EBL may be from about 10 Å to about 1,000 Å. If the thicknesses of the hole transport region HTR, the hole injection layer HIL, the hole transport layer HTL and the electron blocking layer EBL satisfy the above-described ranges, suitable or satisfactory hole transport properties may be achieved without a substantial increase of a driving voltage.

The hole transport region HTR may further include a charge generating material to increase conductivity (e.g., electrical conductivity) in addition to the above-described materials. The charge generating material may be dispersed uniformly or non-uniformly in the hole transport region HTR. The charge generating material may be, for example, a p-dopant. The p-dopant may include at least one selected from metal halide compounds, quinone derivatives, metal oxides, and cyano group-containing compounds, without limitation. For example, the p-dopant may include metal halide compounds such as CuI and/or RbI, quinone derivatives such as tetracyanoquinodimethane (TCNQ) and/or 2,3,5,6-tetrafluoro-7,7′,8,8-tetracyanoquinodimethane (F4-TCNQ), metal oxides such as tungsten oxide and/or molybdenum oxide, cyano group-containing compounds such as dipyrazino[2,3-f: 2′,3′-h] quinoxaline-2,3,6,7,10,11-hexacarbonitrile (HATCN) and/or 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), etc., without limitation.

As described above, the hole transport region HTR may further include a buffer layer in addition to the hole injection layer HIL, the hole transport layer HTL and the electron blocking layer EBL. The buffer layer may compensate resonance distance according to the wavelength of light emitted from an emission layer EML and may increase emission efficiency. The materials included in the buffer layer may use materials which may be included in the hole transport region HTR.

The emission layer EML is provided on the hole transport region HTR. The emission layer EML may have a thickness of, for example, about 100 Å to about 1,000 Å or about 100 Å to about 300 Å. The emission layer EML may have a single layer formed using a single material, a single layer formed using a plurality of different materials, or a multilayer structure having a plurality of layers formed using a plurality of different materials.

In the light emitting element ED of an embodiment, the emission layer EML may emit blue light. The light emitting element ED of an embodiment may include the amine compound of an embodiment in a hole transport region HTR and may show high efficiency and long-life characteristics in a blue emission region. However, embodiments of the present disclosure are not limited thereto.

In the light emitting element ED of an embodiment, the emission layer EML may include anthracene derivatives, pyrene derivatives, fluoranthene derivatives, chrysene derivatives, dihydrobenzanthracene derivatives, and/or triphenylene derivatives. In some embodiments, the emission layer EML may include anthracene derivatives and/or pyrene derivatives.

In the light emitting elements ED of embodiments, shown in FIG. 3 to FIG. 6, the emission layer EML may include a host and a dopant, and the emission layer EML may include a compound represented by Formula E-1 below. The compound represented by Formula E-1 below may be used as a fluorescence host material.

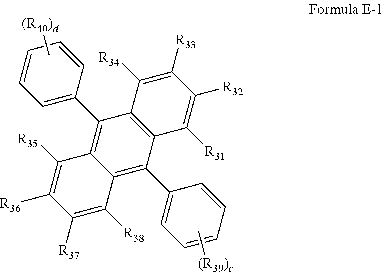

In Formula E-1, R₃₁ to R₄₀ may be each independently a hydrogen atom, a deuterium atom, a halogen atom, a substituted or unsubstituted silyl group, a substituted or unsubstituted thio group, a substituted or unsubstituted oxy group, a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. In some embodiments, R₃₁ to R₄₀ may be combined with an adjacent group to form a saturated hydrocarbon ring, an unsaturated hydrocarbon ring, a saturated heterocycle, or an unsaturated heterocycle.

In Formula E-1, “c” and “d” may be each independently an integer of 0 to 5.

Formula E-1 may be represented by any one selected from among Compound E1 to Compound E19 below.

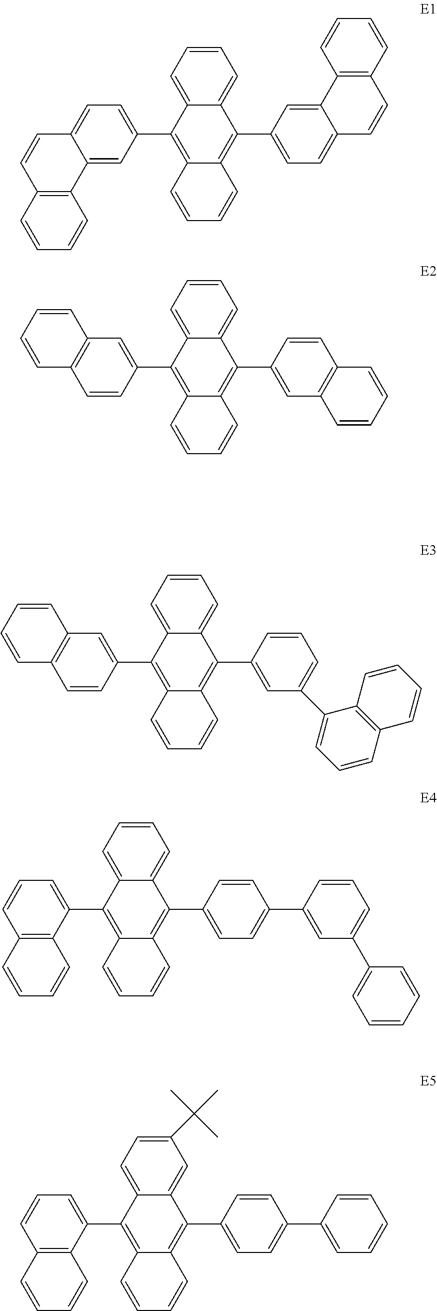

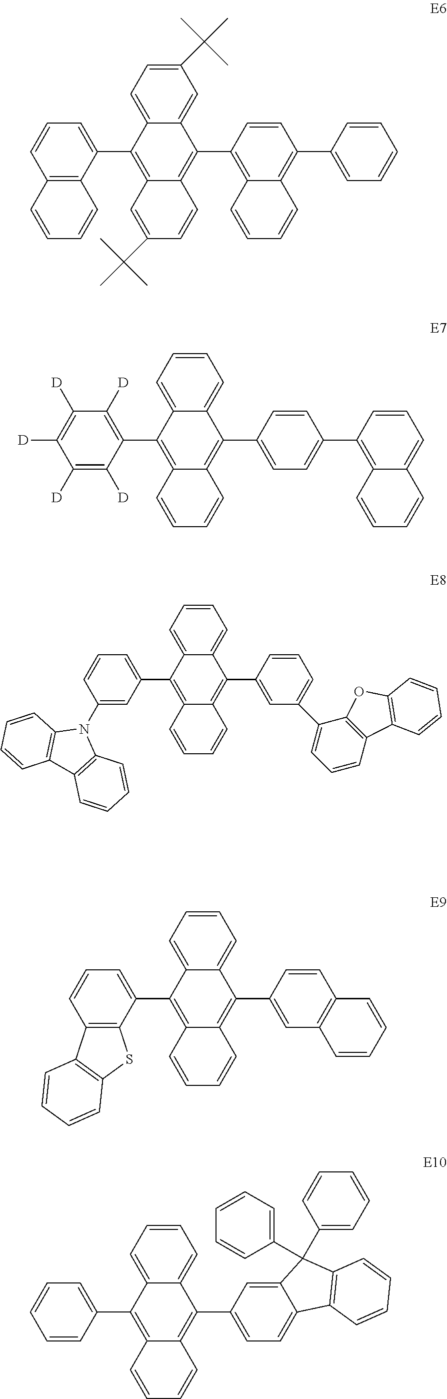

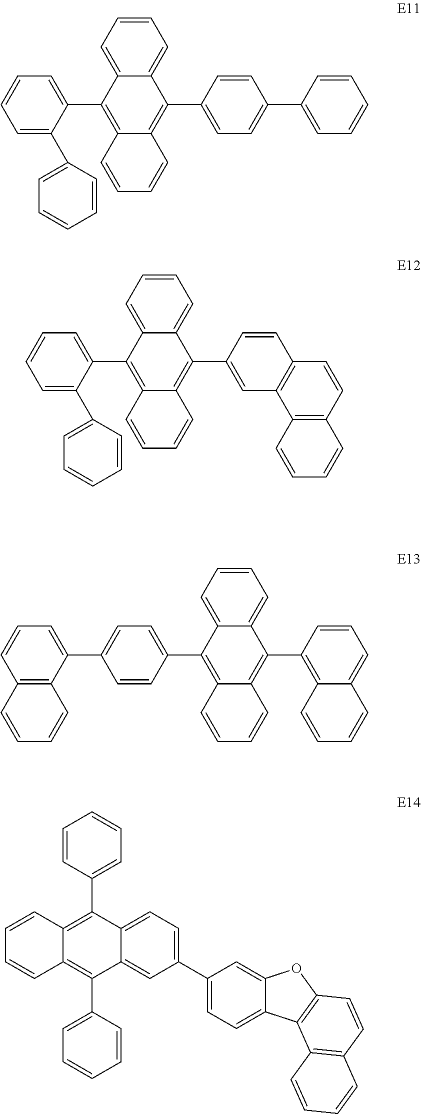

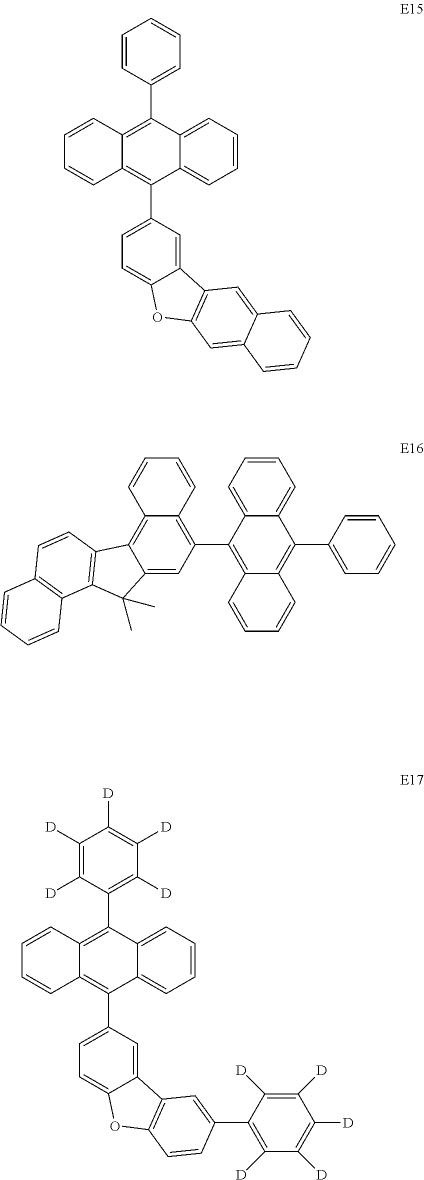

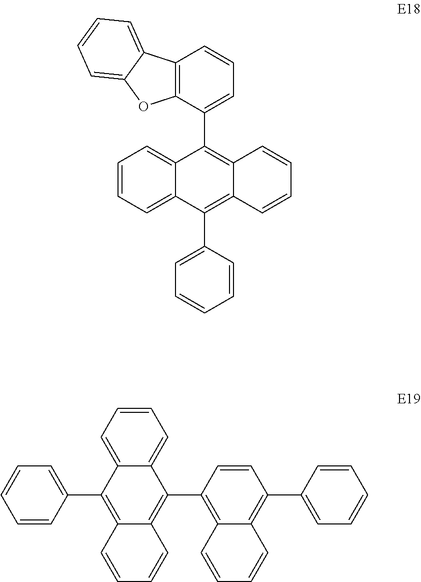

In an embodiment, the emission layer EML may include a compound represented by Formula E-2a or Formula E-2b below. The compound represented by Formula E-2a or Formula E-2b below may be used as a phosphorescence host material.

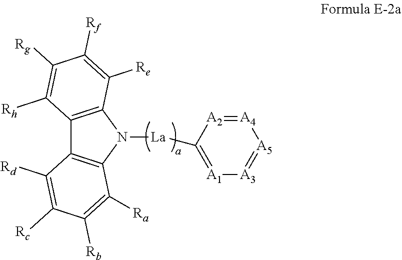

In Formula E-2a, “a” may be an integer of 0 to 10, La may be a direct linkage, a substituted or unsubstituted arylene group of 6 to 30 ring-forming carbon atoms, or a substituted or unsubstituted heteroarylene group of 2 to 30 ring-forming carbon atoms. In some embodiments, if “a” is an integer of 2 or more, a plurality of La may be each independently a substituted or unsubstituted arylene group of 6 to 30 ring-forming carbon atoms, or a substituted or unsubstituted heteroarylene group of 2 to 30 ring-forming carbon atoms.

In addition, in Formula E-2a, A1 to A₅ may be each independently N or CRi. Rₐ to Rᵢ may be each independently a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. Rₐ to Rᵢ may be combined with an adjacent group to form a hydrocarbon ring or a heterocycle including N, O, S, etc. as a ring-forming atom.

In some embodiments, in Formula E-2a, two or three selected from A1 to A5 may be N, and the remainder may be CRᵢ.

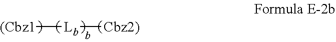

In Formula E-2b, Cbz1 and Cbz2 may be each independently an unsubstituted carbazole group, or a carbazole group substituted with an aryl group of 6 to 30 ring-forming carbon atoms. L_{b} may be a direct linkage, a substituted or unsubstituted arylene group of 6 to 30 ring-forming carbon atoms, or a substituted or unsubstituted heteroarylene group of 2 to 30 ring-forming carbon atoms. “b” is an integer of 0 to 10, and if “b” is an integer of 2 or more, a plurality of L_{b} may be each independently a substituted or unsubstituted arylene group of 6 to 30 ring-forming carbon atoms, or a substituted or unsubstituted heteroarylene group of 2 to 30 ring-forming carbon atoms.

The compound represented by Formula E-2a or Formula E-2b may be represented by any one selected from among the compounds in Compound Group E-2 below. However, the compounds listed in Compound Group E-2 below are only illustrations, and the compound represented by Formula E-2a or Formula E-2b is not limited to the compounds represented in Compound Group E-2 below.

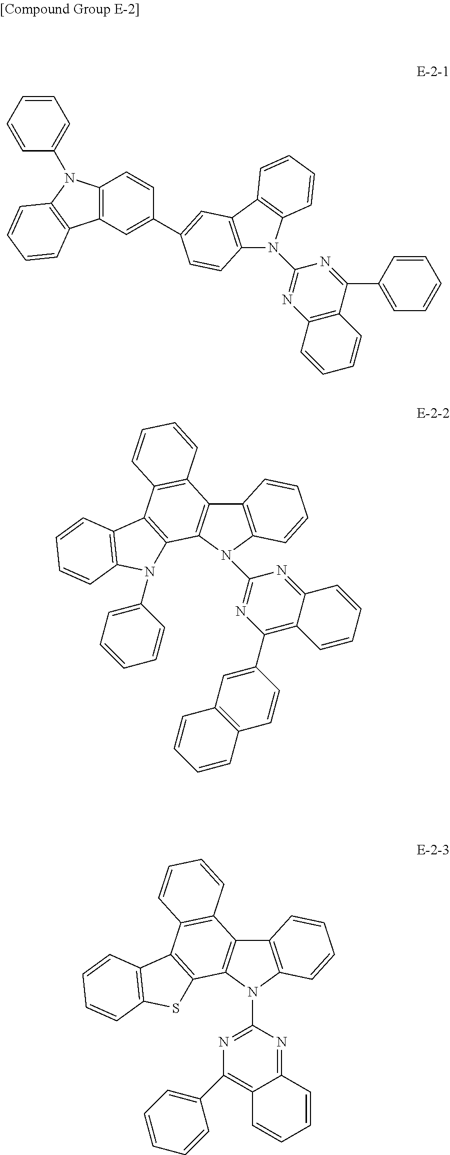 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3c4ccccc4c4c5ccccc5n(-c5nc(-c6ccc7ccccc7c6)c6ccccc6n5)c4c32)cc1 ; c1ccc(-c2nc(-n3c4ccccc4c4c5ccccc5c5c6ccccc6sc5c43)nc3ccccc23)cc1]

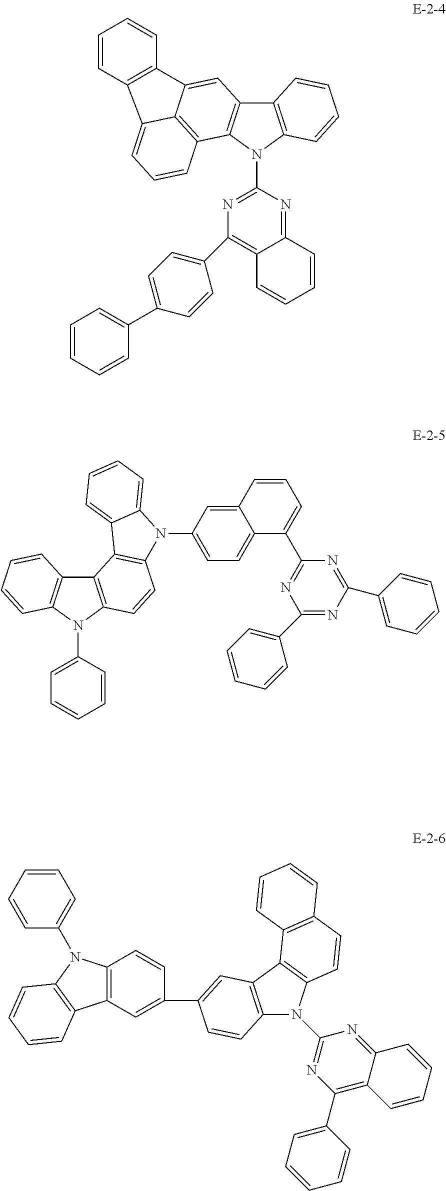 [SMILES: c1ccc(-c2ccc(-c3nc(-n4c5ccccc5c5cc6c7c(cccc7c54)-c4ccccc4-6)nc4ccccc34)cc2)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

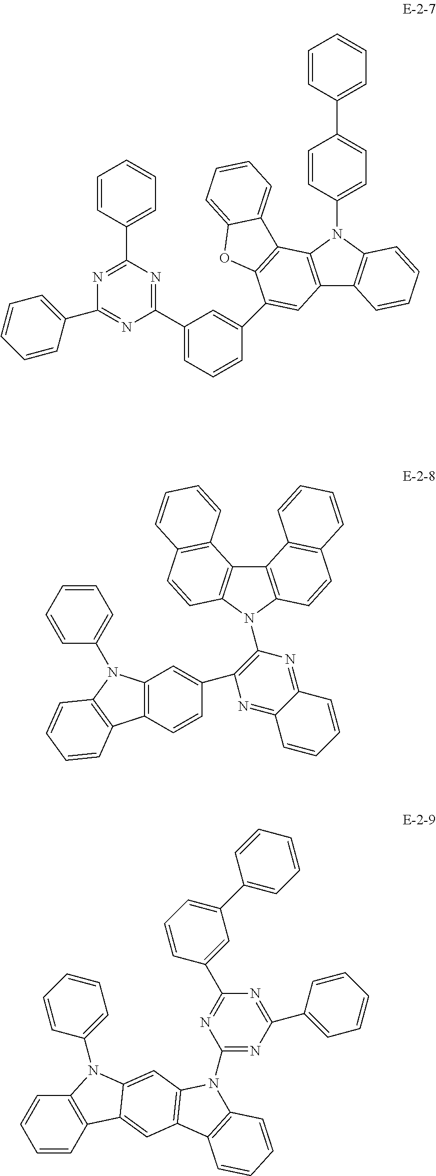 [SMILES: c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1 ; c1ccc(-c2cccc(-c3nc(-c4ccccc4)nc(-n4c5ccccc5c5cc6c7ccccc7n(-c7ccccc7)c6cc54)n3)c2)cc1]

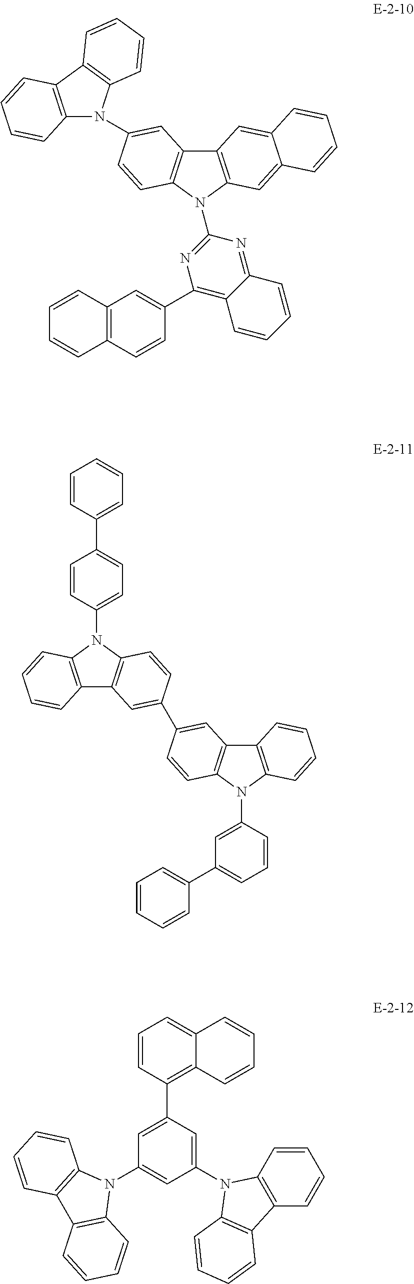 [SMILES: c1ccc2cc(-c3nc(-n4c5ccc(-n6c7ccccc7c7ccccc76)cc5c5cc6ccccc6cc54)nc4ccccc34)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc2c(-c3cc(-n4c5ccccc5c5ccccc54)cc(-n4c5ccccc5c5ccccc54)c3)cccc2c1]

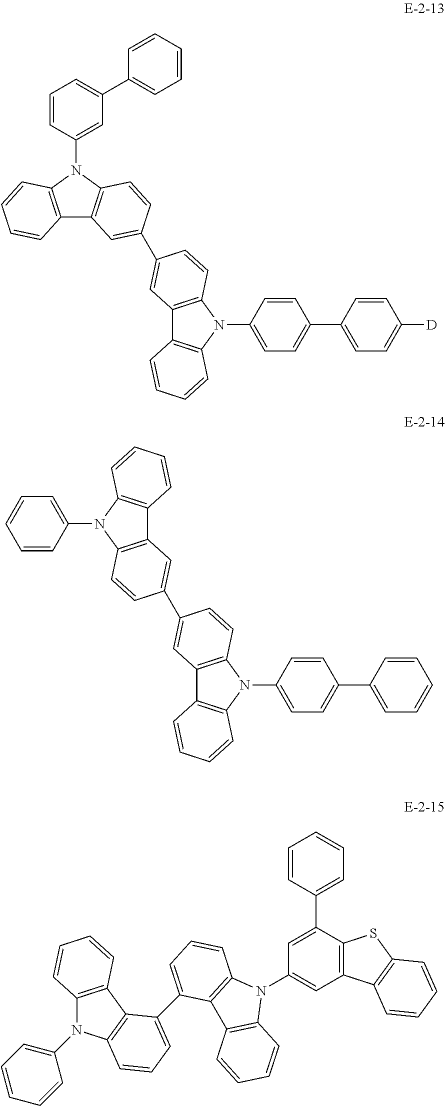 [SMILES: [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

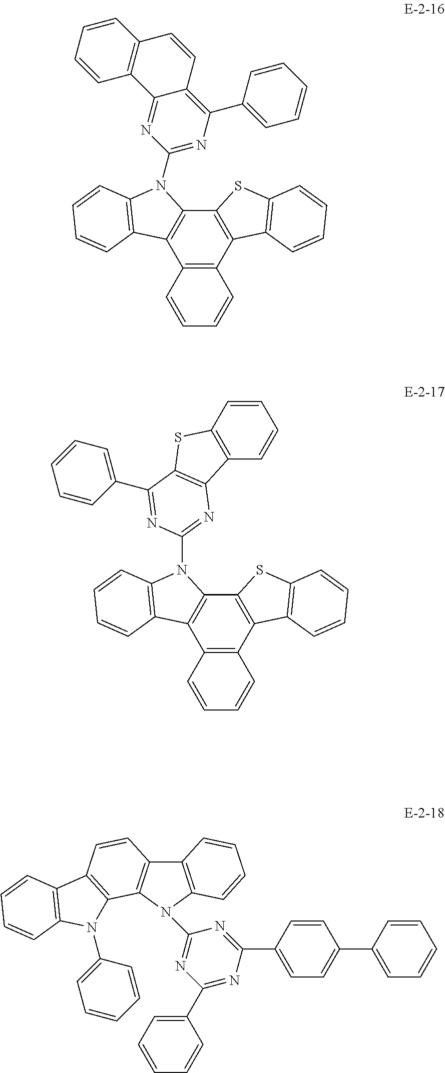 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1]

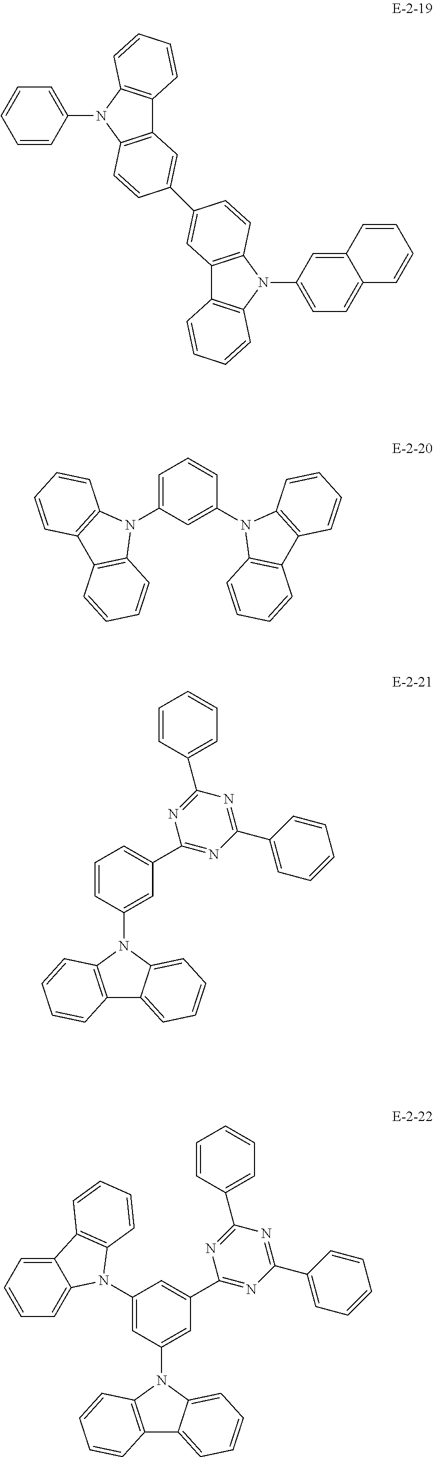 [SMILES: c1ccc(-n2c3ccccc3c3cc(-c4ccc5c(c4)c4ccccc4n5-c4ccc5ccccc5c4)ccc32)cc1 ; c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1]

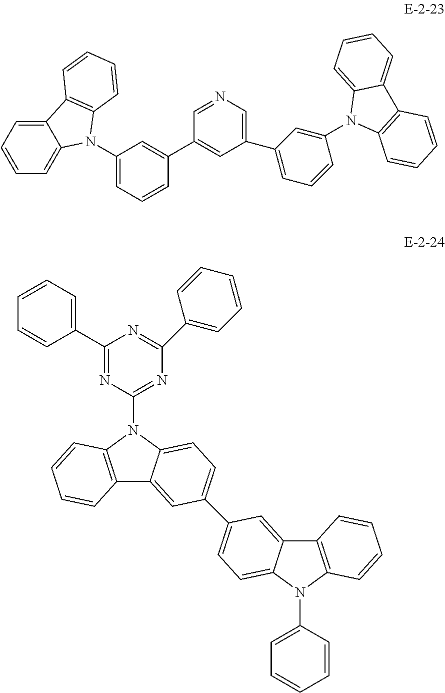 [SMILES: c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include any suitable material generally used in the art as a host material. In some embodiments, the emission layer EML may include as a host material, at least one selected from bis (4-(9H-carbazol-9-yl) phenyl) diphenylsilane (BCPDS), (4-(1-(4-(diphenylamino) phenyl) cyclohexyl) phenyl) diphenyl-phosphine oxide (POPCPA), bis[2-(diphenylphosphino)phenyl] ether oxide (DPEPO), 4,4′-bis(carbazol-9-yl)biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), and 1,3,5-tris(1-phenyl-1H-benzo[d]imidazole-2-yl)benzene (TPBi). However, embodiments of the present disclosure are not limited thereto. For example, tris(8-hydroxyquinolino)aluminum (Alq₃), 9,10-di(naphthalene-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), etc. may be used as the host material.

The emission layer EML may include a compound represented by Formula M-a or Formula M-b below. The compound represented by Formula M-a or Formula M-b may be used as a phosphorescence dopant material. In addition, in an embodiment, the compound represented by Formula M-a or Formula M-b may be used as an auxiliary dopant material.

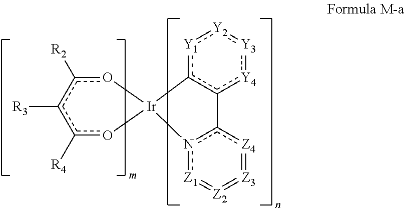

In Formula M-a, Y₁ to Y₄, and Z₁ to Z₄ may be each independently CR₁ or N, and R₁ to R₄ may be each independently a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. In Formula M-a, “m” is 0 or 1, and “n” is 2 or 3. In Formula M-a, if “m” is 0, “n” is 3, and if “m” is 1, “n” is 2.

The compound represented by Formula M-a may be used as a phosphorescence dopant.

The compound represented by Formula M-a may be represented by any one selected from among Compounds M-a1 to M-a25 below. However, Compounds M-a1 to M-a25 below are examples, and the compound represented by Formula M-a is not limited to the compounds represented by Compounds M-a1 to M-a25 below.

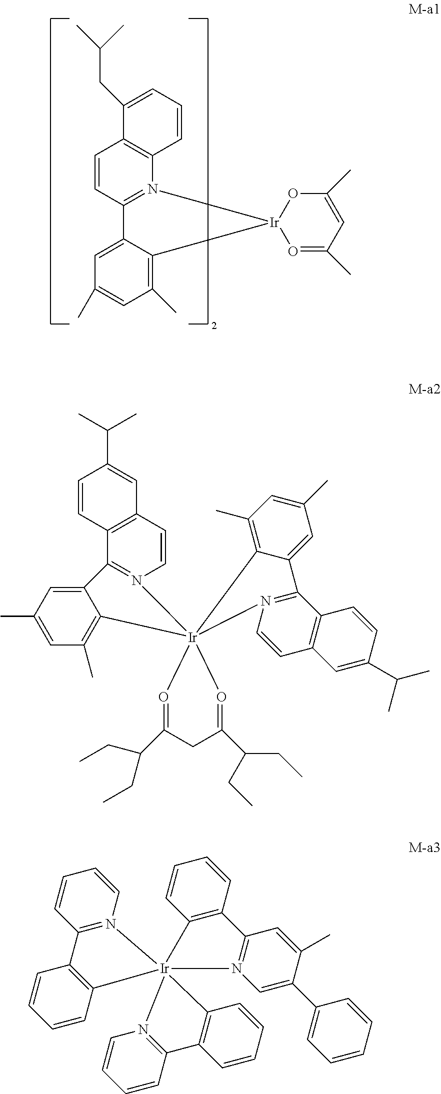

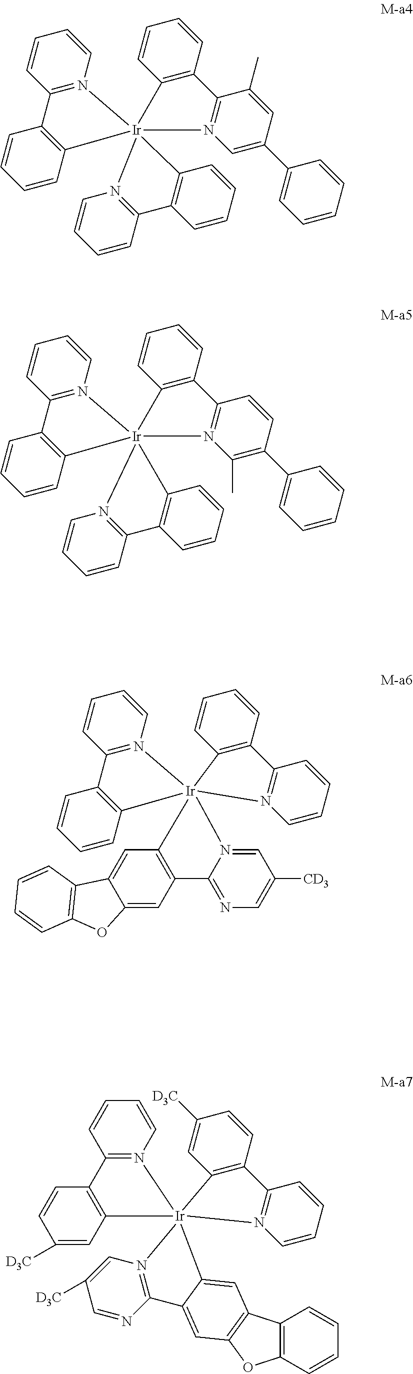

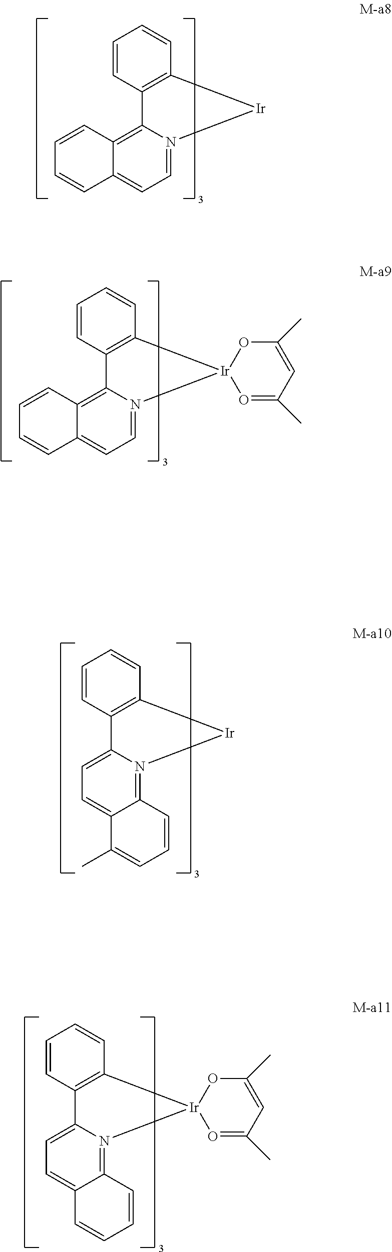

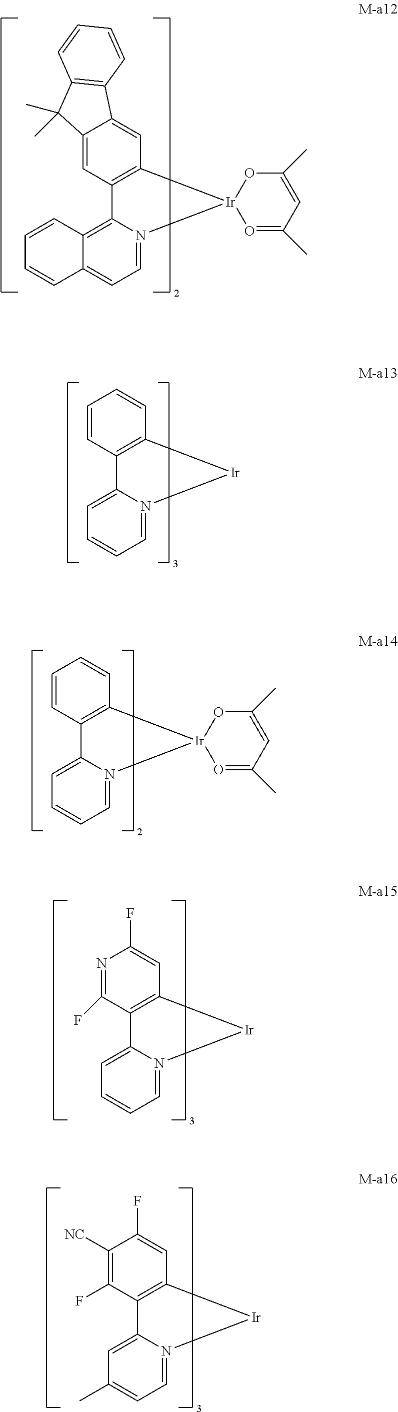

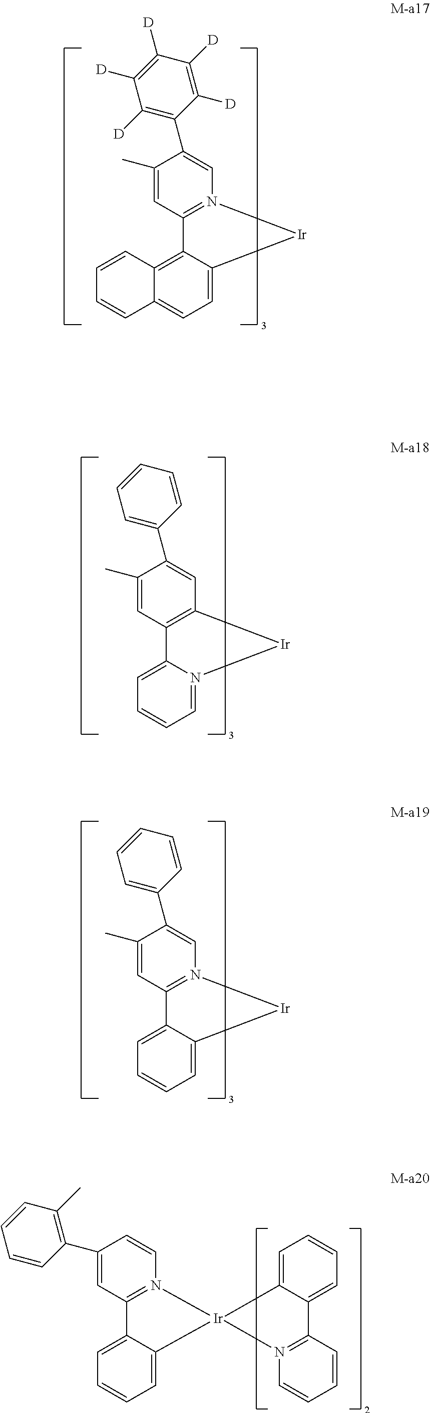

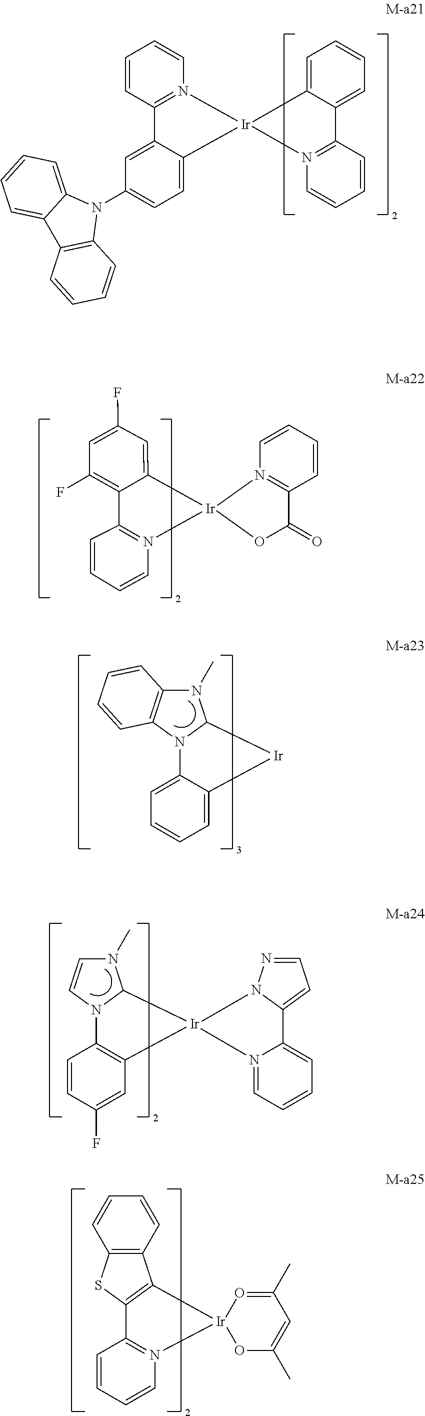

Compound M-a1 and Compound M-a2 may be used as red dopant materials, and Compound M-a3 to Compound M-a7 may be used as green dopant materials.

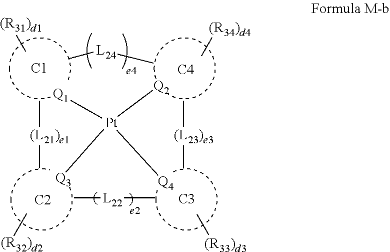

In Formula M-b, Q₁ to Q₄ are each independently C or N, C1 to C4 are each independently a substituted or unsubstituted hydrocarbon ring of 5 to 30 ring-forming carbon atoms, or a substituted or unsubstituted heterocycle of 2 to 30 ring-forming carbon atoms. L₂₁ to L₂₄ are each independently a direct linkage, *—O—*, *—S—*,

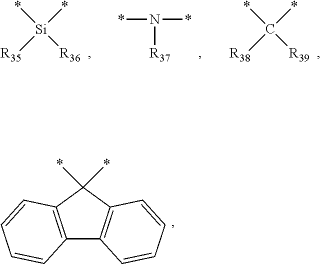

a substituted or unsubstituted divalent alkyl group of 1 to 20 carbon atoms, a substituted or unsubstituted arylene group of 6 to 30 ring-forming carbon atoms, or a substituted or unsubstituted heteroarylene group of 2 to 30 ring-forming carbon atoms, and e1 to e4 are each independently 0 or 1. R₃₁ to R₃₉ are each independently a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring, and d1 to d4 are each independently an integer of 0 to 4.

The compound represented by Formula M-b may be used as a blue phosphorescence dopant or a green phosphorescence dopant. In addition, the compound represented by Formula M-b may be an auxiliary dopant in an embodiment and may be further included in the emission layer EML.

The compound represented by Formula M-b may be represented by any one selected from among Compound M-b-1 to Compound M-b-11 below. However, the compounds below are examples, and the compound represented by Formula M-b is not limited to Compound M-b-1 to Compound M-b-11 below.

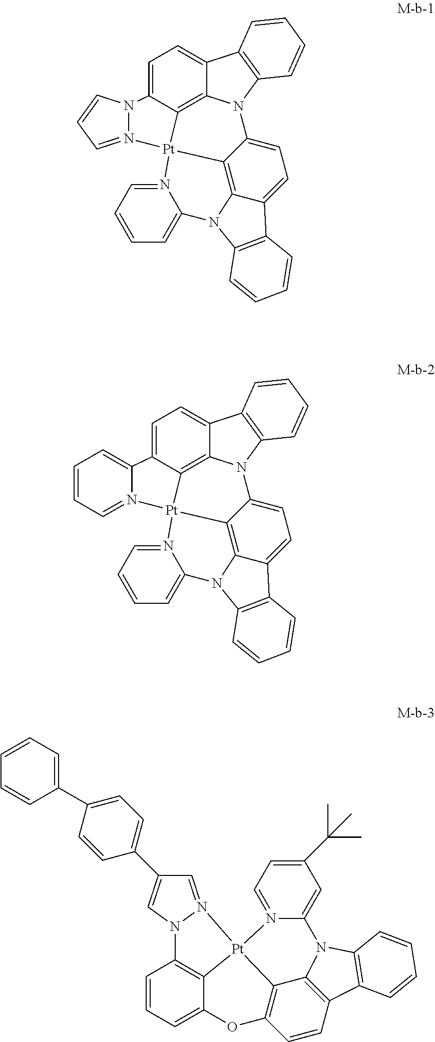

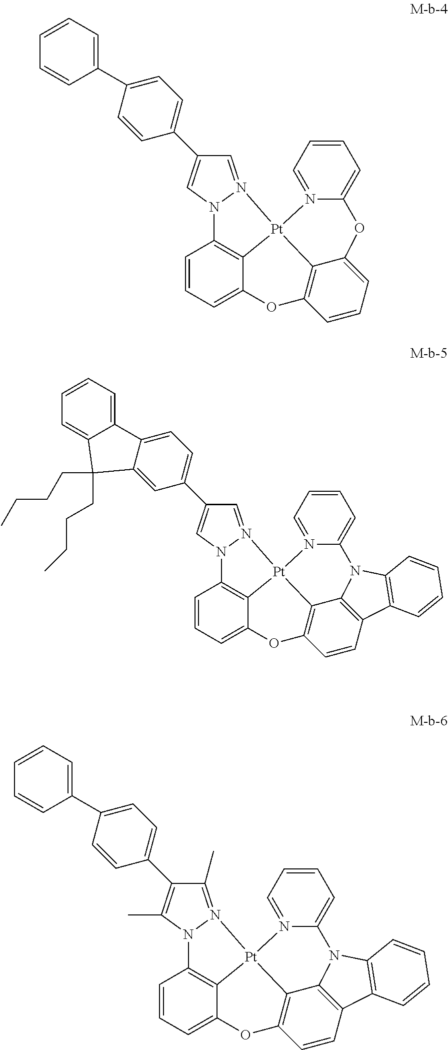

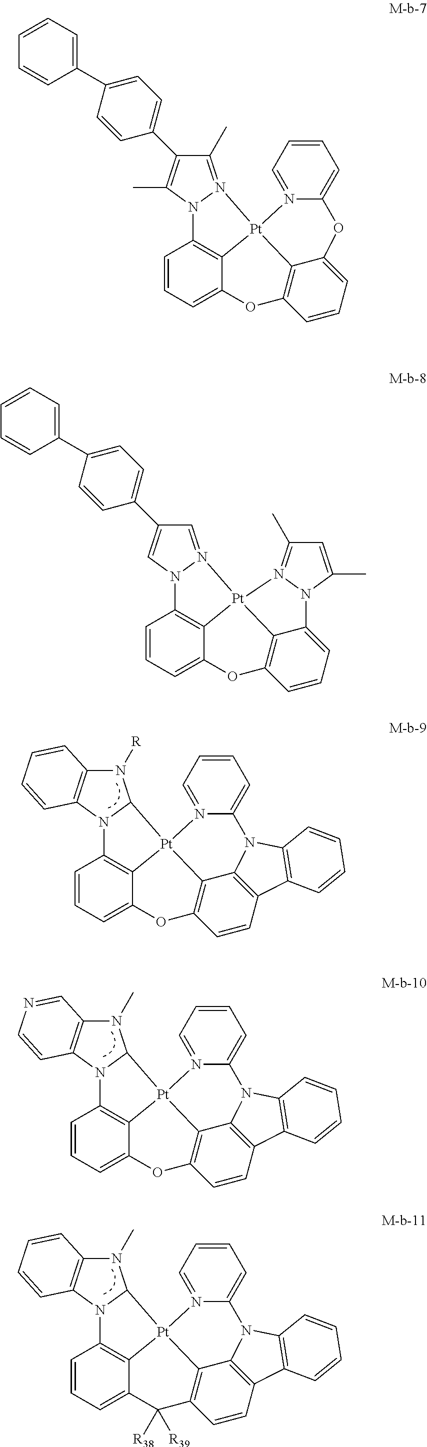

In the compounds above, R, R₃₈, and R₃₉ may be each independently a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

The emission layer EML may include any one selected from among Formula F-a to Formula F-c below. The compounds represented by Formula F-a to Formula F-c below may be used as fluorescence dopant materials.

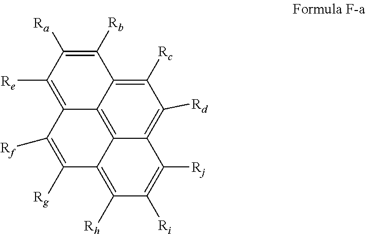

In Formula F-a, two selected from Rₐ to Rⱼ may be each independently substituted with *—NAr₁Ar₂. The remainder not substituted with *—NAr₁Ar₂ among Rₐ to Rⱼ may be each independently a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In *—NAr₁Ar₂, Ar₁ and Ar₂ may be each independently a substituted or unsubstituted aryl group of 6 to 30 ring-forming carbon atoms, or a substituted or unsubstituted heteroaryl group of 2 to 30 ring-forming carbon atoms. For example, at least one selected from among Ar₁ and Ar₂ may be a heteroaryl group including O or S as a ring-forming atom.

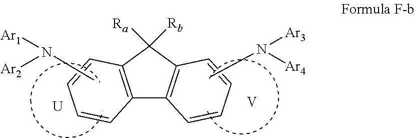

In Formula F-b, Rₐ and Rb may be each independently a hydrogen atom, a deuterium atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring.

In Formula F-b, Ar₁ to Ar₄ may be each independently a substituted or unsubstituted aryl group of 6 to 30 ring-forming carbon atoms, or a substituted or unsubstituted heteroaryl group of 2 to 30 ring-forming carbon atoms.

In Formula F-b, U and V may be each independently a substituted or unsubstituted hydrocarbon ring of 5 to 30 ring-forming carbon atoms, or a substituted or unsubstituted heterocycle of 2 to 30 ring-forming carbon atoms.

In Formula F-b, the number of rings represented by U and V may be each independently 0 or 1. For example, in Formula F-b, if the number of U or V is 1, one ring forms a fused ring at the designated part by U or V, and if the number of U or V is 0, a ring is not present at the designated part by U or V. In some embodiments, if the number of U is 0, and the number of V is 1, or if the number of U is 1, and the number of V is 0, a fused ring having the fluorene core of Formula F-b may be a ring compound having four rings. In addition, if the number of both U and V is 0, the fused ring of Formula F-b may be a ring compound having three rings. In addition, if the number of both U and V is 1, a fused ring having the fluorene core of Formula F-b may be a ring compound having five rings.

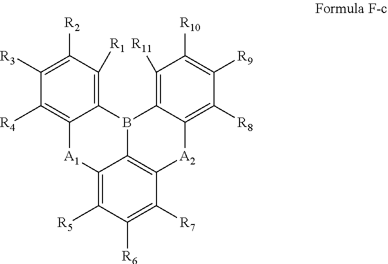

In Formula F-c, A1 and A2 may be each independently O, S, Se, or NRₘ, and Rₘ may be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. R₁ to R₁₁ are each independently a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring.

In Formula F-c, A1 and A2 may be each independently combined with the substituents of an adjacent ring to form a fused ring. For example, if A1 and A2 may be each independently NRₘ, A1 may be combined with R₄ or R₅ to form a ring. In addition, A2 may be combined with R₇ or R₈ to form a ring.

In an embodiment, the emission layer EML may include any suitable dopant material generally used in the art. In some embodiments, the emission layer EML may include styryl derivatives (for example, 1,4-bis[2-(3-N-ethylcarbazoryl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (N-BDAVBi), and/or 4,4′-bis[2-(4-(N,N-diphenylamino)phenyl)vinyl]biphenyl (DPAVBi)), perylene and/or derivatives thereof (for example, 2,5,8,11-tetra-t-butylperylene (TBP)), pyrene and/or derivatives thereof (for example, 1,1-dipyrene, 1,4-dipyrenylbenzene, and/or 1,4-bis(N,N-diphenylamino)pyrene), etc.

In an embodiment, if a plurality of emission layers EML are included, at least one emission layer EML may include any suitable phosphorescence dopant material generally used in the art. For example, the phosphorescence dopant may use a metal complex including iridium (Ir), platinum (Pt), osmium (Os), gold (Au), titanium (Ti), zirconium (Zr), hafnium (Hf), europium (Eu), terbium (Tb) or thulium (Tm). In some embodiments, iridium(III) bis(4,6-difluorophenylpyridinato-N,C2′)picolinate (Firpic), bis(2,4-difluorophenylpyridinato)-tetrakis(1-pyrazolyl)borate iridium(III) (Fir6), and/or platinum octaethyl porphyrin (PtOEP) may be used as the phosphorescence dopant. However, embodiments of the present disclosure are not limited thereto.

In some embodiments, the emission layer EML may include a hole transport host and an electron transport host. In addition, the emission layer EML may include an auxiliary dopant and a light emitting dopant. As an example, the auxiliary dopant may include a phosphorescence dopant material or a thermally activated delayed fluorescence dopant. In some embodiments, the emission layer EML may include a hole transport host, an electron transport host, an auxiliary dopant, and a light emitting dopant.

In addition, an exciplex may be formed by the hole transport host and the electron transport host in the emission layer EML. In this case, the triplet energy of the exciplex formed by the hole transport host and the electron transport host may correspond to T1 which is a gap between the LUMO energy level of the electron transport host and the HOMO energy level of the hole transport host.

In some embodiments, the triplet energy (T1) of the exciplex formed by the hole transport host and the electron transport host may be about 2.4 eV to about 3.0 eV. In addition, the triplet energy of the exciplex may be a value smaller than the energy gap of each host material. Accordingly, the exciplex may have a triplet energy of about 3.0 eV or less, which is the energy gap between the hole transport host and the electron transport host.

In some embodiments, at least one emission layer EML may include a quantum dot material. The core of the quantum dot may be selected from Group II-VI compounds, Group III-VI compounds, Group I-III-VI compounds, Group III-V compounds, Group III-II-V compounds, Group IV-VI compounds, Group IV elements, Group IV compounds, and combinations thereof.

The Group II-VI compound may be selected from the group consisting of: a binary compound selected from the group consisting of CdSe, CdTe, CdS, ZnS, ZnSe, ZnTe, ZnO, HgS, HgSe, HgTe, MgSe, MgS, and mixtures thereof; a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and mixtures thereof; and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, HgZnSTe, and mixtures thereof.

The Group III-VI compound may include a binary compound such as In₂S₃, and/or In₂Se₃, a ternary compound such as InGaS₃, and/or InGaSe₃, or optional combinations thereof.

The Group I-III-VI compound may be selected from a ternary compound selected from the group consisting of AgInS, AgInS₂, CuInS, CuInS₂, AgGaS₂, CuGaS₂, CuGaO₂, AgGaO₂, AgAlO₂ and mixtures thereof, and/or a quaternary compound such as AgInGaS₂, and CuInGaS₂.

The Group III-V compound may be selected from the group consisting of a binary compound selected from the group consisting of GaN, GaP, GaAs, GaSb, AlN, AlP, AlAs, AlSb, InN, InP, InAs, InSb, and mixtures thereof, a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and mixtures thereof, and a quaternary compound selected from the group consisting of GaAlNP, GaAlNAs, GaAlNSb, GaAlPAs, GaAlPSb, GaInNP, GaInNAs, GalnNSb, GaInPAs, GalnPSb, InAlNP, InAlNAs, InAlNSb, InAlPAs, InAlPSb, and mixtures thereof. In some embodiments, the Group III-V compound may further include a Group II metal. For example, InZnP, etc. may be selected as a Group III-II-V compound.

The Group IV-VI compound may be selected from the group consisting of a binary compound selected from the group consisting of SnS, SnSe, SnTe, PbS, PbSe, PbTe, and mixtures thereof, a ternary compound selected from the group consisting of SnSeS, SnSeTe, SnSTe, PbSeS, PbSeTe, PbSTe, SnPbS, SnPbSe, SnPbTe, and mixtures thereof, and a quaternary compound selected from the group consisting of SnPbSSe, SnPbSeTe, SnPbSTe, and mixtures thereof. The IV group element may be selected from the group consisting of Si, Ge, and a mixture thereof. The Group IV compound may be a binary compound selected from the group consisting of SiC, SiGe, and a mixture thereof.

In this case, the binary compound, the ternary compound or the quaternary compound may be present at uniform (e.g., substantially uniform) concentration in a particle or may be present at a partially different concentration distribution state in the same particle. In addition, a core/shell structure in which one quantum dot wraps another quantum dot may be possible. The interface of the core and the shell may have a concentration gradient in which the concentration of an element present in the shell is decreased along a direction toward the center of the quantum dot.

In some embodiments, the quantum dot may have the above-described core-shell structure including a core including a nanocrystal and a shell wrapping the core. The shell of the quantum dot may play the role of a protection layer for preventing or reducing the chemical deformation of the core to maintain semiconductor properties and/or a charging layer for imparting the quantum dot with electrophoretic properties. The shell may have a single layer or a multilayer. Examples of the shell of the quantum dot may include a metal and/or non-metal oxide, a semiconductor compound, or combinations thereof.

For example, the metal and/or non-metal oxide may include a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄ and/or NiO, and/or a ternary compound such as MgAl₂O₄, CoFe₂O₄, NiFe₂O₄ and/or CoMn₂O₄, but embodiments of the present disclosure are not limited thereto.

Also, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but embodiments of the present disclosure are not limited thereto.

The quantum dot may have a full width of half maximum (FWHM) of emission wavelength spectrum of about 45 nm or less, about 40 nm or less, or, for example, about 30 nm or less. Within this range, color purity and/or color reproducibility may be improved. In addition, light emitted via such quantum dot is emitted in all (e.g., substantially all) directions, and light viewing angle properties may be improved.

In addition, the shape of the quantum dot may be any suitable shape generally used in the art, without specific limitation. For example, the shape of spherical, pyramidal, multi-arm, and/or cubic nanoparticle, nanotube, nanowire, nanofiber, nanoplate particle, etc. may be used.

The quantum dot may control the color of light emitted according to the particle size, and accordingly, the quantum dot may have various suitable emission colors such as blue, red, and green.

In the light emitting elements ED of embodiments, as shown in FIG. 3 to FIG. 6, the electron transport region ETR is provided on the emission layer EML. The electron transport region ETR may include at least one selected from of a hole blocking layer HBL, an electron transport layer ETL and an electron injection layer EIL. However, embodiments of the present disclosure are not limited thereto.

The electron transport region ETR may have a single layer formed using a single material, a single layer formed using a plurality of different materials, or a multilayer structure having a plurality of layers formed using a plurality of different materials.

For example, the electron transport region ETR may have a single layer structure of an electron injection layer EIL or an electron transport layer ETL, or a single layer structure formed using an electron injection material and an electron transport material. Further, the electron transport region ETR may have a single layer structure formed using a plurality of different materials, or a structure stacked from the emission layer EML of electron transport layer ETL/electron injection layer EIL, hole blocking layer HBL/electron transport layer ETL/electron injection layer EIL, electron transport layer ETL/buffer layer/electron injection layer EIL, without limitation. The thickness of the electron transport region ETR may be, for example, from about 1,000 Å to about 1,500 Å.

The electron transport region ETR may be formed using various suitable methods such as a vacuum deposition method, a spin coating method, a cast method, a Langmuir-Blodgett (LB) method, an inkjet printing method, a laser printing method, and/or a laser induced thermal imaging (LITI) method.

The electron transport region ETR may include a compound represented by Formula ET-1 below.

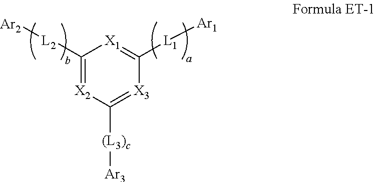

In Formula ET-1, at least one selected from among X₁ to X₃ is N, and the remainder are CRₐ. Rₐ may be a hydrogen atom, a deuterium atom, a substituted or unsubstituted alkyl of 1 to 20 carbon atoms, a substituted or unsubstituted aryl group of 6 to 30 ring-forming carbon atoms, or a substituted or unsubstituted heteroaryl group of 2 to 30 ring-forming carbon atoms. Ar₁ to Ar₃ may be each independently a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In Formula ET-1, “a” to “c” may be each independently an integer of 0 to 10. In Formula ET-1, L₁ to L₃ may be each independently a direct linkage, a substituted or unsubstituted arylene group of 6 to 30 ring-forming carbon atoms, or a substituted or unsubstituted heteroarylene group of 2 to 30 ring-forming carbon atoms. In some embodiments, if “a” to “c” are integers of 2 or more, L₁ to L₃ may be each independently a substituted or unsubstituted arylene group of 6 to 30 ring-forming carbon atoms, or a substituted or unsubstituted heteroarylene group of 2 to 30 ring-forming carbon atoms.

The electron transport region ETR may include an anthracene-based compound. However, embodiments of the present disclosure are not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazolyl-1-ylphenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), berylliumbis(benzoquinolin-10-olate (Bebq₂), 9,10-di(naphthalene-2-yl)anthracene (ADN), 1,3-bis[3,5-di(pyridin-3-yl)phenyl]benzene (BmPyPhB), diphenyl(4-(triphenylsilyl)phenyl)phosphine oxide (TSPO1), and mixtures thereof, without limitation.

The electron transport region ETR may include at least one selected from among Compounds ET1 to ET36 below.

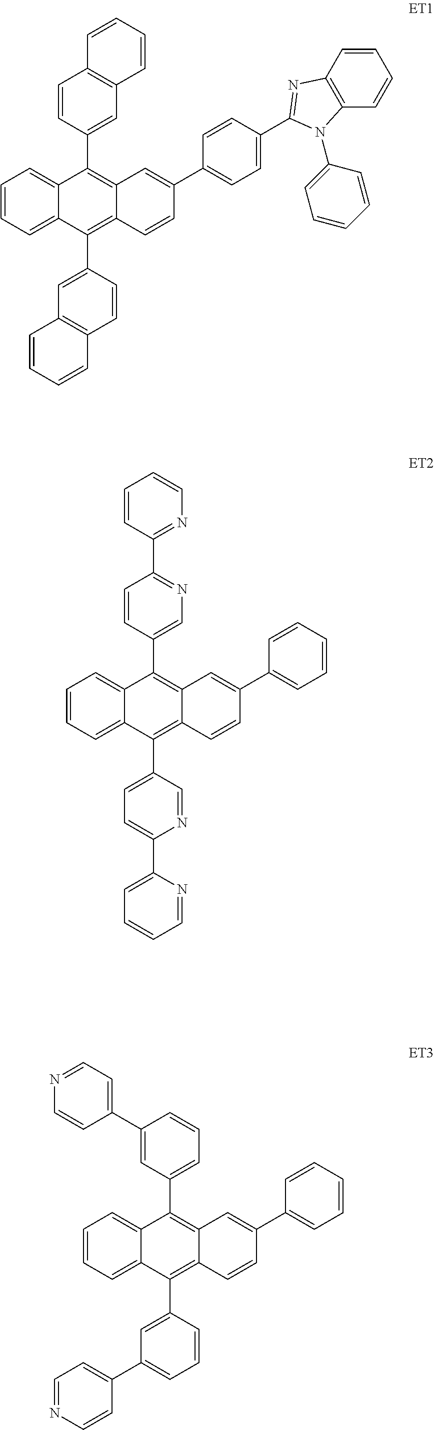 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

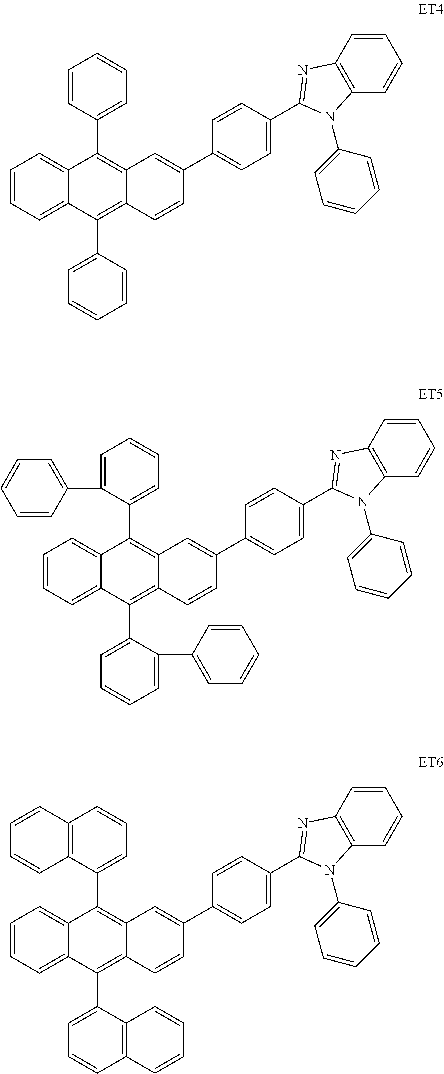 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

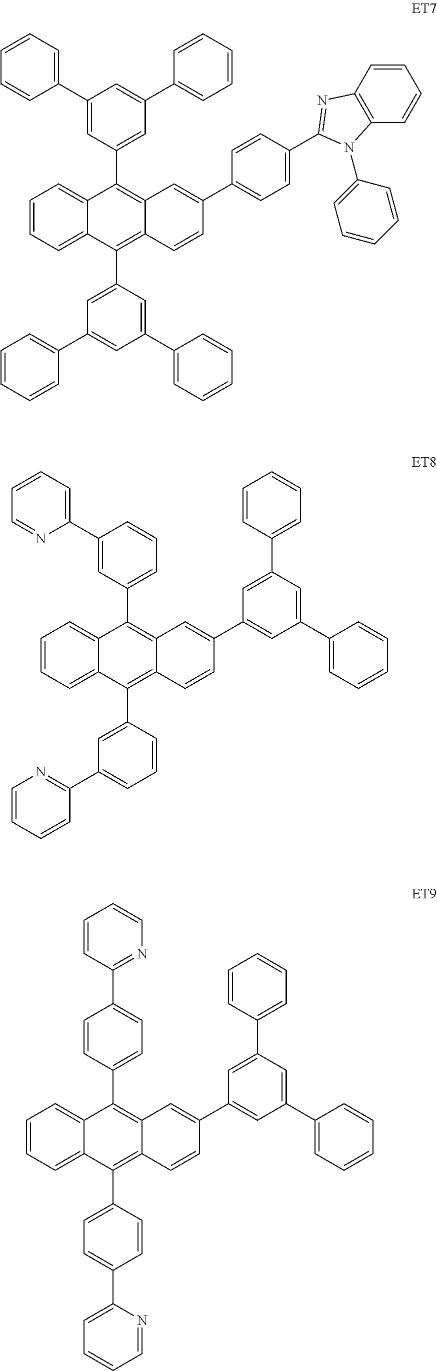 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

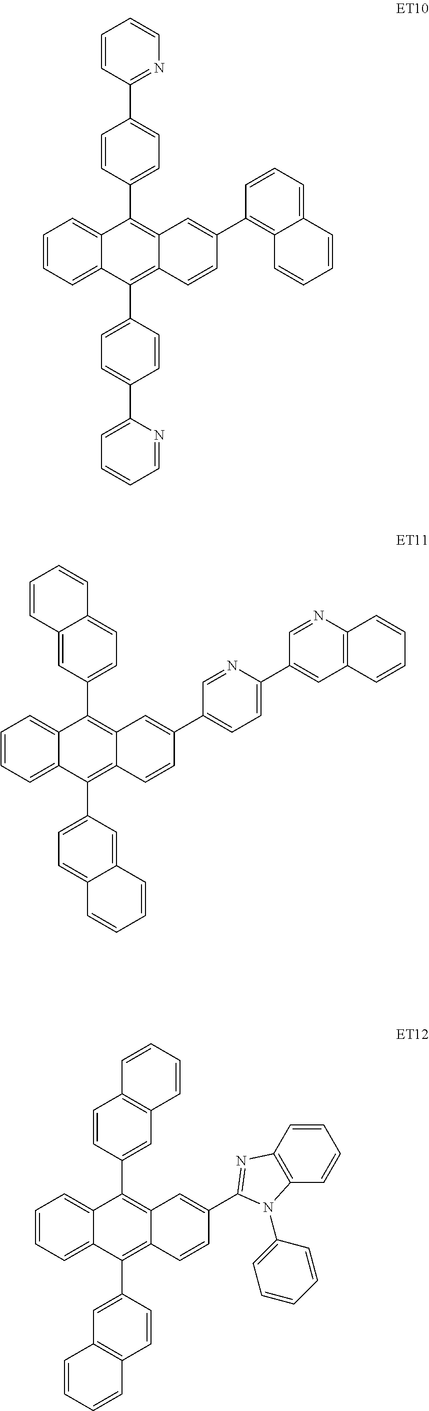 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

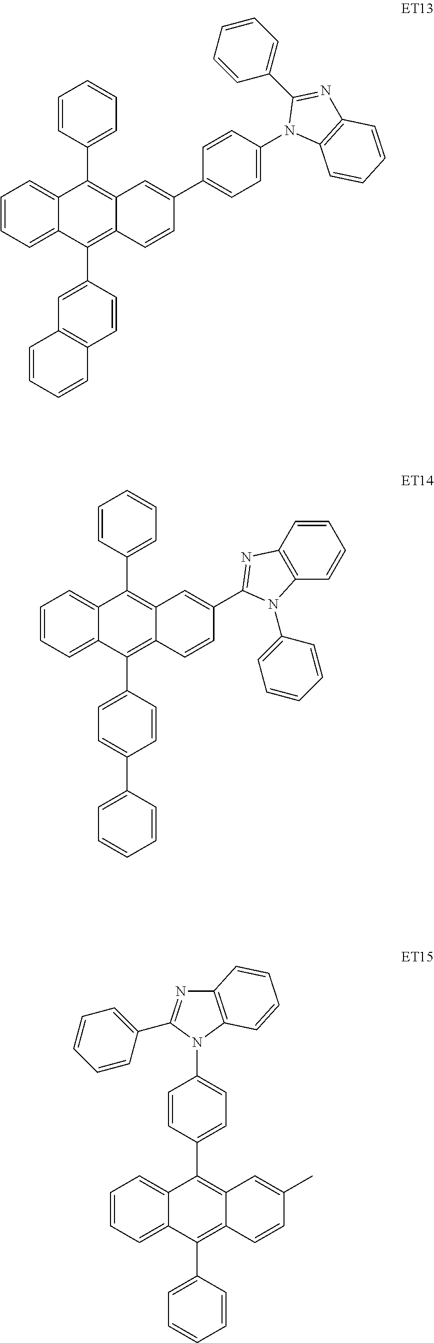 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

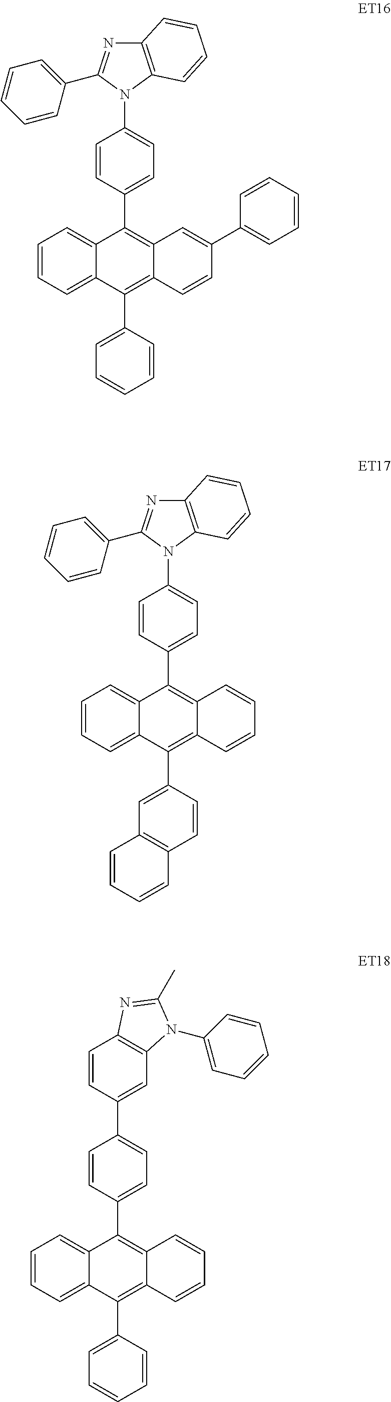 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

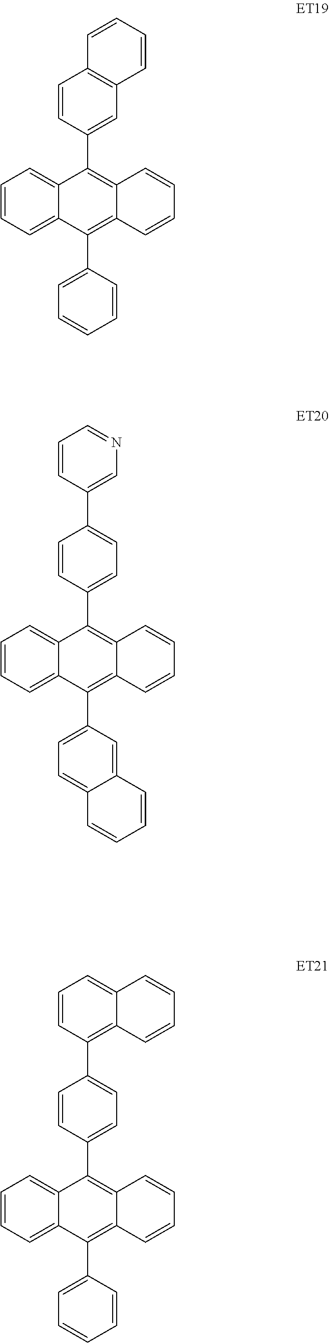

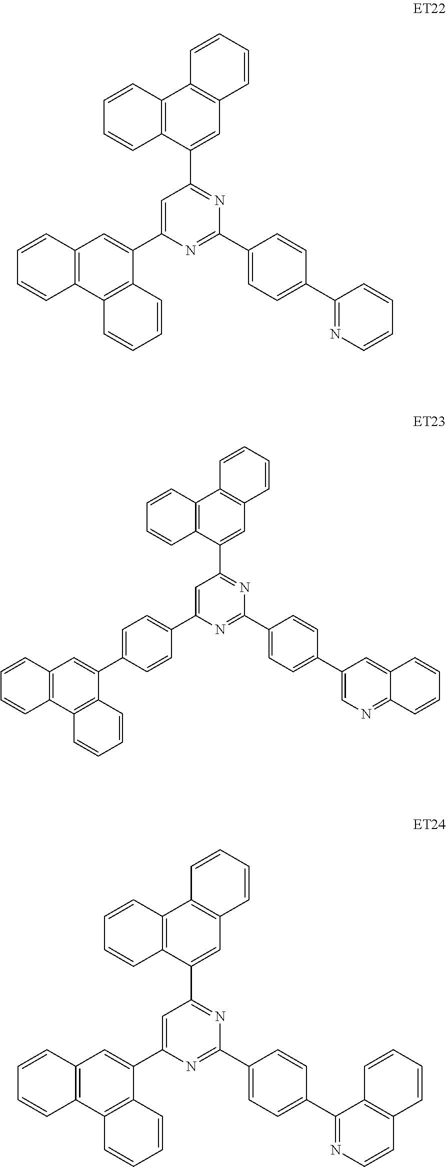 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

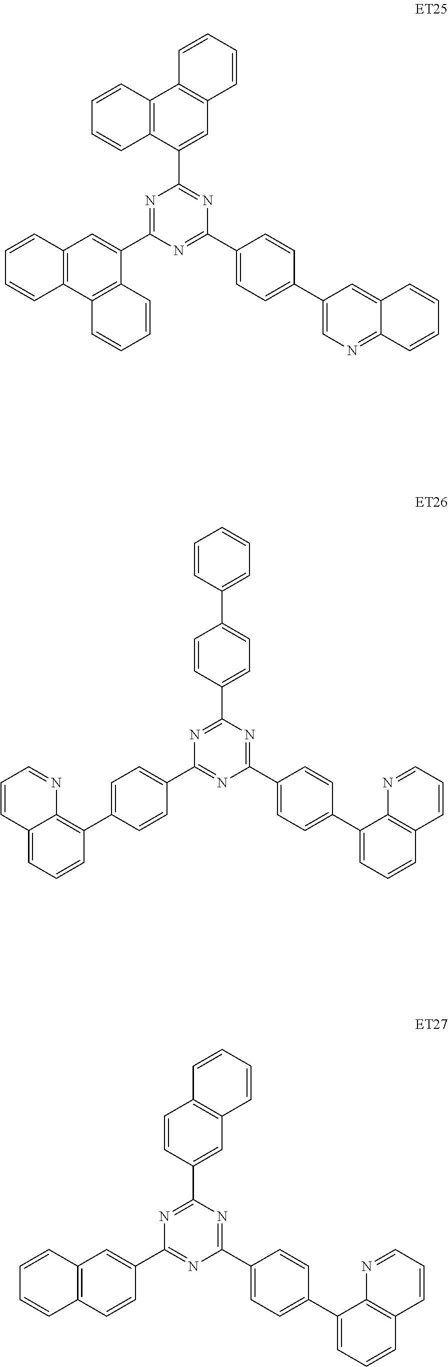 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

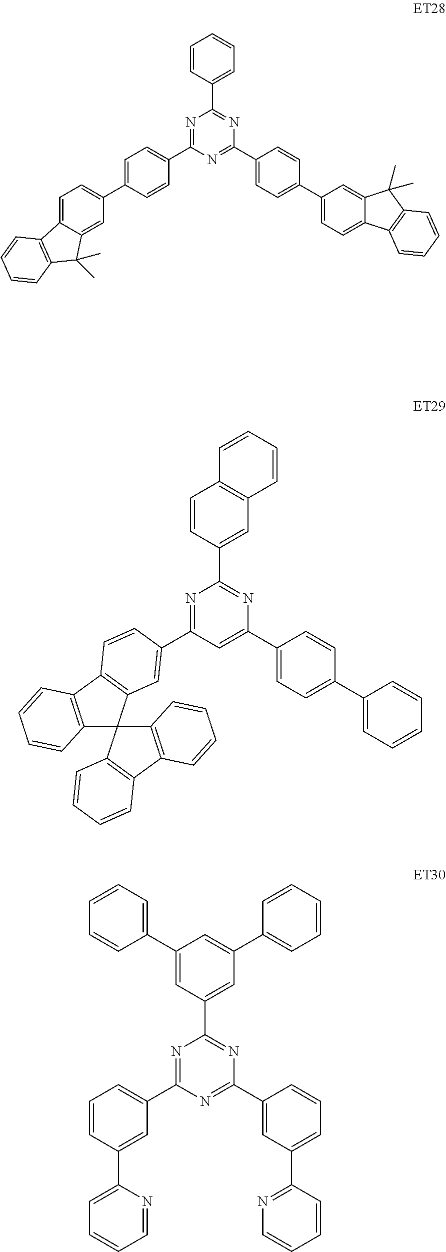 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

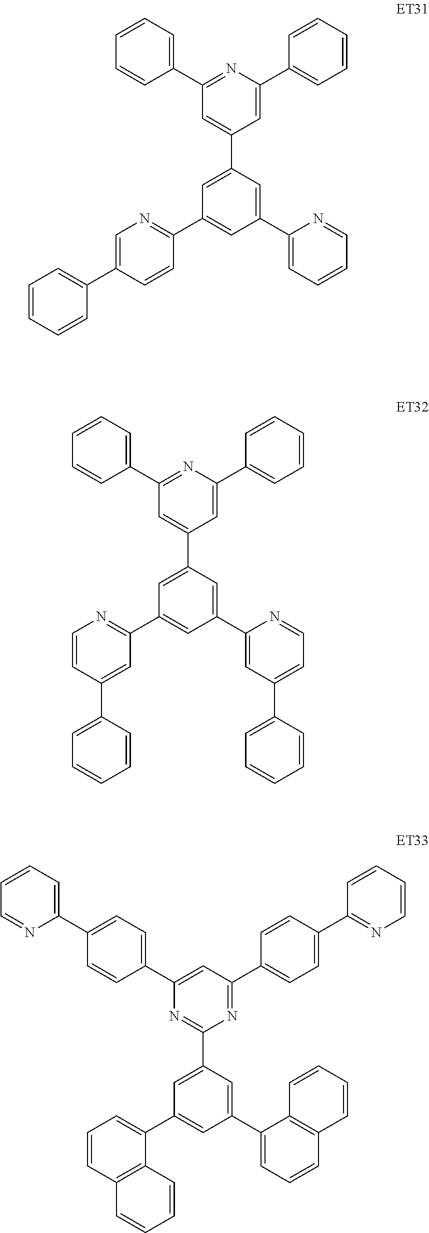 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

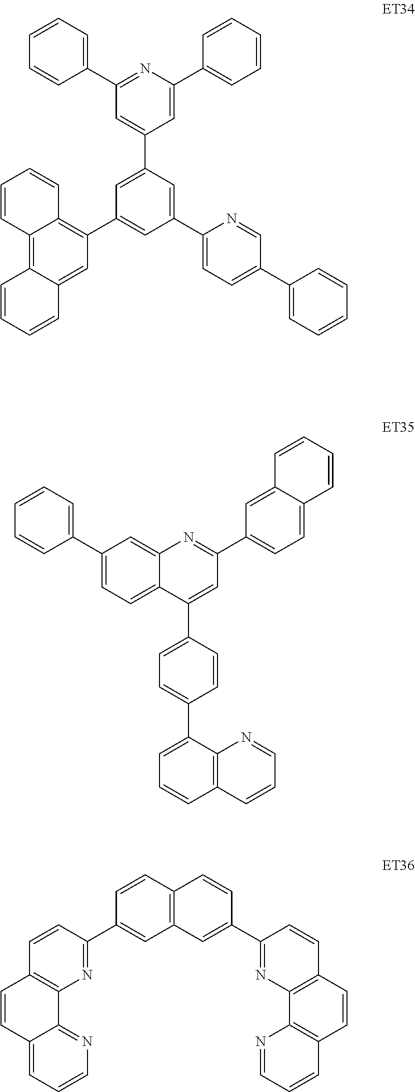 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In addition, the electron transport region ETR may include a metal halide such as LiF, NaCl, CsF, RbCl, RbI, CuI and/or KI, a metal in lanthanides such as Yb, and/or a co-depositing material of the metal halide and the metal in lanthanides. For example, the electron transport region ETR may include KI:Yb, RbI:Yb, LiF:Yb, etc., as the co-depositing material. In some embodiments, the electron transport region ETR may use a metal oxide such as Li₂O and/or BaO, and/or 8-hydroxy-lithium quinolate (Liq). However, embodiments of the present disclosure are not limited thereto. The electron transport region ETR also may be formed using a mixture material of an electron transport material and an insulating organo metal salt. The organo metal salt may be a material having an energy band gap of about 4 eV or more. In some embodiments, the organo metal salt may include, for example, metal acetates, metal benzoates, metal acetoacetates, metal acetylacetonates, and/or metal stearates.

The electron transport region ETR may include at least one selected from of 2,9-dimethyl-4,7-diphenyl-1,10-phenanthroline (BCP), diphenyl(4-(triphenylsilyl)phenyl)phosphine oxide (TSPO1) and 4,7-diphenyl-1,10-phenanthroline (Bphen) in addition to the aforementioned materials. However, embodiments of the present disclosure are not limited thereto.

The electron transport region ETR may include the compounds of the electron transport region in at least one selected from among an electron injection layer EIL, an electron transport layer ETL, and a hole blocking layer HBL.

If the electron transport region ETR includes the electron transport layer ETL, the thickness of the electron transport layer ETL may be from about 100 Å to about 1,000 Å, for example, from about 150 Å to about 500 Å. If the thickness of the electron transport layer ETL satisfies the above-described range, suitable or satisfactory electron transport properties may be obtained without substantial increase of a driving voltage. If the electron transport region ETR includes the electron injection layer EIL, the thickness of the electron injection layer EIL may be from about 1 Å to about 100 Å, and from about 3 Å to about 90 Å. If the thickness of the electron injection layer EIL satisfies the above described range, suitable or satisfactory electron injection properties may be obtained without inducing substantial increase of a driving voltage.

The second electrode EL2 is provided on the electron transport region ETR. The second electrode EL2 may be a common electrode. The second electrode EL2 may be a cathode or an anode, but embodiments of the present disclosure are not limited thereto. For example, if the first electrode EL1 is an anode, the second cathode EL2 may be a cathode, and if the first electrode EL1 is a cathode, the second electrode EL2 may be an anode. The second electrode EL2 may include at least one selected from among Ag, Mg, Cu, Al, Pt, Pd, Au, Ni, Nd, Ir, Cr, Li, Ca, LiF, Mo, Ti, W, In, Sn, and Zn, compounds of two or more selected therefrom, mixtures of two or more selected therefrom, and/or oxides thereof.

The second electrode EL2 may be a transmissive electrode, a transflective electrode or a reflective electrode. If the second electrode EL2 is the transmissive electrode, the second electrode EL2 may include a transparent metal oxide, for example, ITO, IZO, ZnO, ITZO, etc.

If the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca (stacked structure of LiF and Ca), LiF/Al (stacked structure of LiF and Al), Mo, Ti, Yb, W, compounds including thereof, and/or mixtures thereof (for example, AgMg, or AgYb). In some embodiments, the second electrode EL2 may have a multilayered structure including a reflective layer or a transflective layer formed using the above-described materials and a transparent conductive layer formed using ITO, IZO, ZnO, ITZO, etc. For example, the second electrode EL2 may include the aforementioned metal materials, combinations of two or more metal materials selected from the aforementioned metal materials, and/or oxides of the aforementioned metal materials.

In some embodiments, the second electrode EL2 may be connected with an auxiliary electrode. If the second electrode EL2 is connected with the auxiliary electrode, the resistance of the second electrode EL2 may decrease.

A capping layer CPL may be further on the second electrode EL2 in the light emitting element ED of an embodiment. The capping layer CPL may include a multilayer or a single layer.

In an embodiment, the capping layer CPL may be an organic layer or an inorganic layer. For example, if the capping layer CPL includes an inorganic material, the inorganic material may include an alkali metal compound such as LiF, an alkaline earth metal compound such as SiON, SiNx, SiOy, etc.

For example, if the capping layer CPL includes an organic material, the organic material may include α-NPD, NPB, TPD, m-MTDATA, Alq₃, CuPc, N4,N4,N4′,N4′-tetra(biphenyl-4-yl) biphenyl-4,4′-diamine (TPD15), 4,4′,4″-tris(carbazol sol-9-yl) triphenylamine (TCTA), etc., and/or include an epoxy resin, and/or acrylate such as methacrylate. In addition, a capping layer CPL may include at least one selected from among Compounds P1 to P5 below, but embodiments of the present disclosure are not limited thereto.

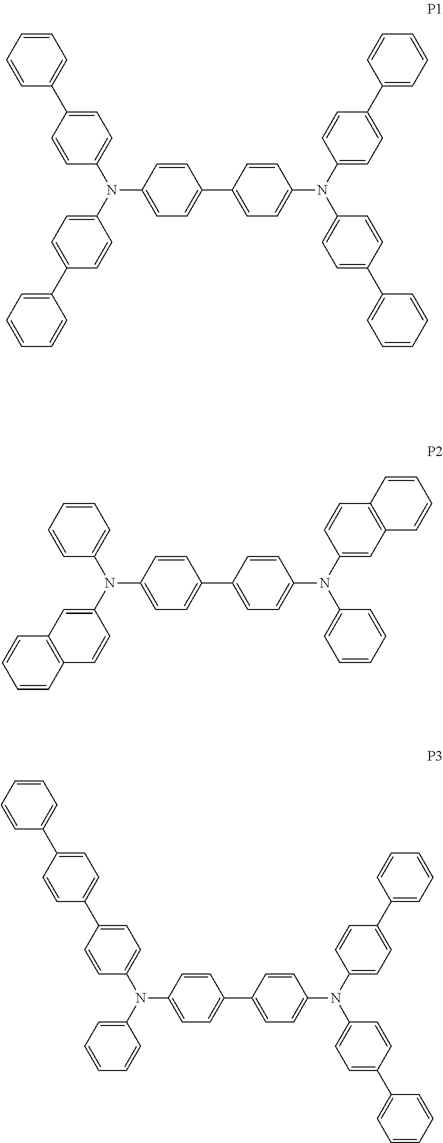 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

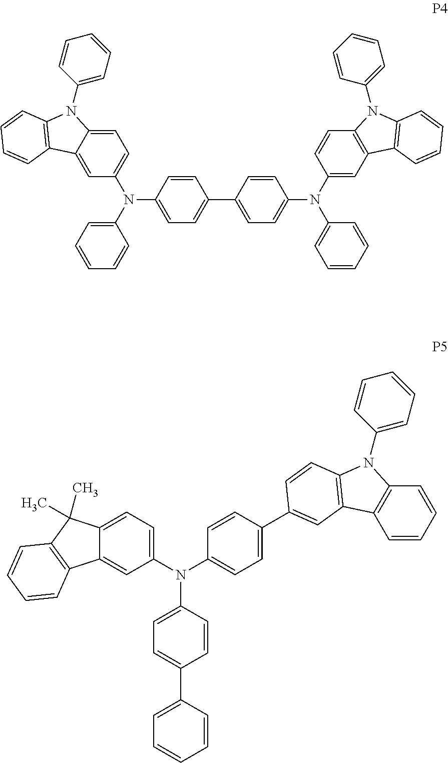 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

In some embodiments, the refractive index of the capping layer CPL may be about 1.6 or more. For example, the refractive index of the capping layer CPL with respect to light in a wavelength range of about 550 nm to about 660 nm may be about 1.6 or more.

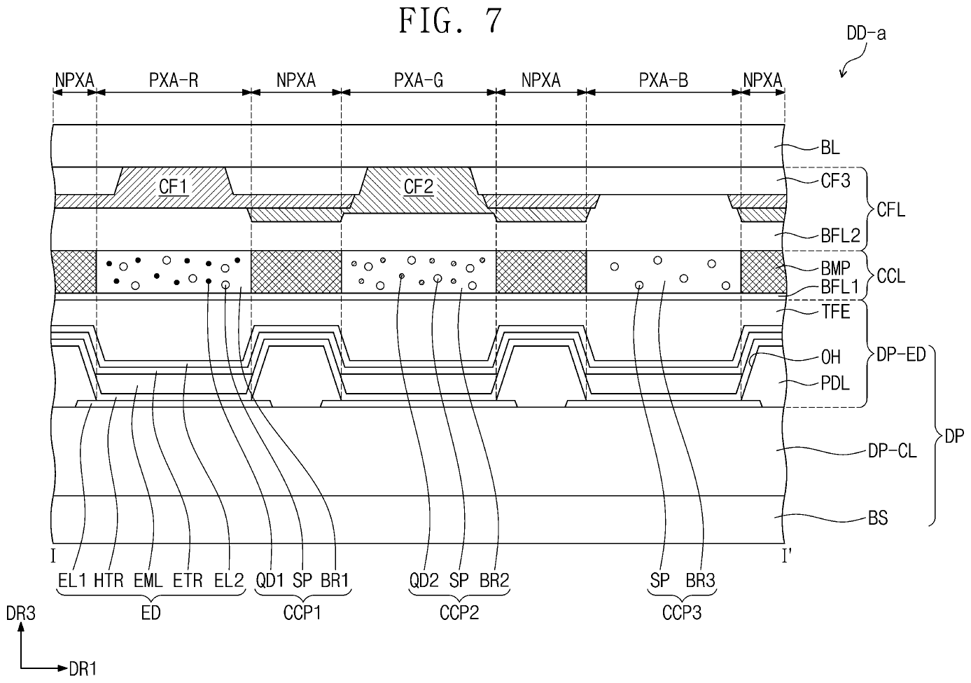

FIG. 7 to FIG. 10 are cross-sectional views on display devices according to embodiments. Hereinafter, in the explanation on the display devices of embodiments, referring to FIG. 7 to FIG. 10, the overlapping parts with the explanation with respect to FIG. 1 to FIG. 6 will not be repeated here, and the different features will be explained chiefly.

Referring to FIG. 7, a display device DD-a according to an embodiment may include a display panel DP including a display element layer DP-ED, a light controlling layer CCL on the display panel DP, and a color filter layer CFL.

In an embodiment shown in FIG. 7, the display panel DP includes a base layer BS, a circuit layer DP-CL provided on the base layer BS and a display element layer DP-ED, and the display element layer DP-ED may include a light emitting element ED.

The light emitting element ED may include a first electrode EL1, a hole transport region HTR on the first electrode EL1, an emission layer EML on the hole transport region HTR, an electron transport region ETR on the emission layer EML, and a second electrode EL2 on the electron transport region ETR. In some embodiments, the structures of the light emitting elements of FIG. 3 to FIG. 6 may be applied to the structure of the light emitting element ED shown in FIG. 7.

The hole transport region HTR of the light emitting element ED included in the display device DD-a according to an embodiment may include the amine compound of an embodiment.

Referring to FIG. 7, the emission layer EML may be in an opening part OH defined in a pixel definition layer PDL. For example, the emission layer EML divided by the pixel definition layer PDL and correspondingly provided to each of luminous areas PXA-R, PXA-G and PXA-B may emit light in the same wavelength region. In the display device DD-a of an embodiment, the emission layer EML may emit blue light. In some embodiments, different from the drawings, in an embodiment, the emission layer EML may be provided as a common layer for all luminous areas PXA-R, PXA-G and PXA-B.

The light controlling layer CCL may be on the display panel DP. The light controlling layer CCL may include a light converter. The light converter may be a quantum dot and/or a phosphor. The light converter may transform the wavelength of light provided and then emit. For example, the light controlling layer CCL may be a layer including a quantum dot and/or a layer including a phosphor.

The light controlling layer CCL may include a plurality of light controlling parts CCP1, CCP2 and CCP3. The light controlling parts CCP1, CCP2 and CCP3 may be separated (e.g., spaced apart) from one another.

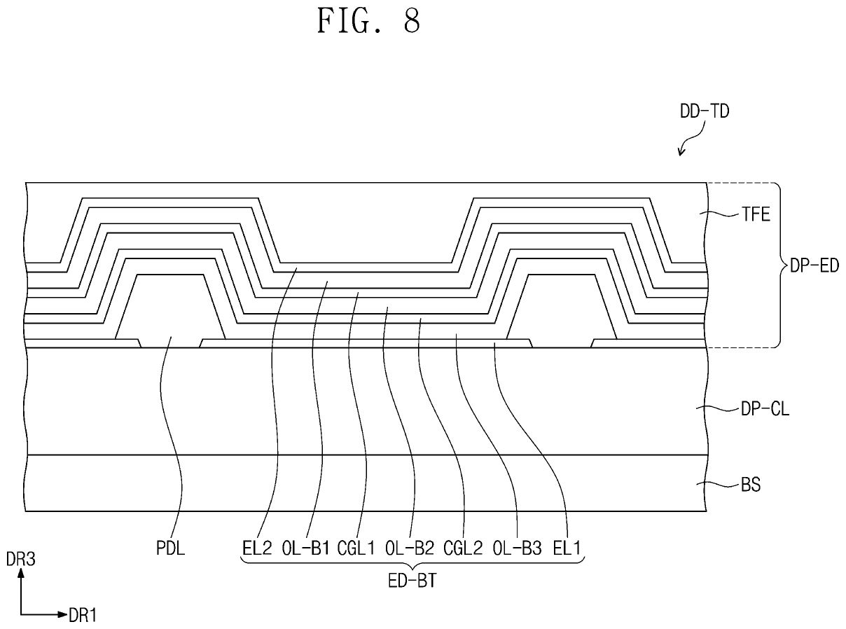

Referring to FIG. 7, a partition pattern BMP may be between the separated light controlling parts CCP1, CCP2 and CCP3, but embodiments of the present disclosure are not limited thereto. In FIG. 8, the partition pattern BMP is shown not to be overlapped with the light controlling parts CCP1, CCP2 and CCP3, but at least a portion of the edge of the light controlling parts CCP1, CCP2 and CCP3 may be overlapped with the partition pattern BMP.

The light controlling layer CCL may include a first light controlling part CCP1 including a first quantum dot QD1 that converts a first color light provided from the light emitting element ED into a second color light, a second light controlling part CCP2 including a second quantum dot QD2 that converts the first color light into a third color light, and a third light controlling part CCP3 that transmits the first color light.

In an embodiment, the first light controlling part CCP1 may provide red light which is the second color light, and the second light controlling part CCP2 may provide green light which is the third color light. The third color controlling part CCP3 may transmit and provide blue light which is the first color light provided from the light emitting element ED. For example, the first quantum dot QD1 may be a red quantum dot, and the second quantum dot QD2 may be a green quantum dot. For the quantum dots QD1 and QD2, the same contents as those described above may be applied.

In addition, the light controlling layer CCL may further include a scatterer SP (e.g., a light scatterer SP). The first light controlling part CCP1 may include the first quantum dot QD1 and the scatterer SP, the second light controlling part CCP2 may include the second quantum dot QD2 and the scatterer SP, and the third light controlling part CCP3 may not include a quantum dot but include the scatterer SP.

The scatterer SP may be an inorganic particle. For example, the scatterer SP may include at least one selected from among TiO₂, ZnO, Al₂O₃, SiO₂, and hollow silica. The scatterer SP may include at least one selected from among TiO₂, ZnO, Al₂O₃, SiO₂, and hollow silica, or may be a mixture of two or more materials selected from among TiO₂, ZnO, Al₂O₃, SiO₂, and hollow silica.

Each of the first light controlling part CCP1, the second light controlling part CCP2, and the third light controlling part CCP3 may include base resins BR1, BR2 and BR3 that disperse the quantum dots QD1 and QD2 and the scatterer SP. In an embodiment, the first light controlling part CCP1 may include the first quantum dot QD1 and the scatterer SP dispersed in the first base resin BR1, the second light controlling part CCP2 may include the second quantum dot QD2 and the scatterer SP dispersed in the second base resin BR2, and the third light controlling part CCP3 may include the scatterer particle SP dispersed in the third base resin BR3. The base resins BR1, BR2 and BR3 are mediums in which the quantum dots QD1 and QD2 and the scatterer SP are dispersed, and may be composed of various suitable resin compositions which may be generally referred to as a binder. For example, the base resins BR1, BR2 and BR3 may be acrylic resins, urethane-based resins, silicone-based resins, epoxy-based resins, etc. The base resins BR1, BR2 and BR3 may be transparent resins. In an embodiment, the first base resin BR1, the second base resin BR2 and the third base resin BR3 may be the same or different from each other.

The light controlling layer CCL may include a barrier layer BFL1. The barrier layer BFL1 may play the role of blocking or reducing the penetration of moisture and/or oxygen (hereinafter, may be referred to as “humidity/oxygen”). The barrier layer BFL1 may be on the light controlling parts CCP1, CCP2 and CCP3 and may block or reduce the exposure of the light controlling parts CCP1, CCP2 and CCP3 to humidity/oxygen. In some embodiments, the barrier layer BFL1 may cover the light controlling parts CCP1, CCP2 and CCP3. In addition, the barrier layer BFL2 may be provided between the light controlling parts CCP1, CCP2 and CCP3 and a color filter layer CFL.

The barrier layers BFL1 and BFL2 may include at least one inorganic layer. For example, the barrier layers BFL1 and BFL2 may be formed by including an inorganic material. For example, the barrier layers BFL1 and BFL2 may be formed by including silicon nitride, aluminum nitride, zirconium nitride, titanium nitride, hafnium nitride, tantalum nitride, silicon oxide, aluminum oxide, titanium oxide, tin oxide, cerium oxide and/or silicon oxynitride and/or a metal thin film that has light transmittance. In some embodiments, the barrier layers BFL1 and BFL2 may further include an organic layer. The barrier layers BFL1 and BFL2 may be composed of a single layer of a plurality of layers.

In the display device DD-a of an embodiment, the color filter layer CFL may be on the light controlling layer CCL. For example, the color filter layer CFL may be directly on the light controlling layer CCL. In this case, the barrier layer BFL2 may be omitted.

The color filter layer CFL may include filters CF1, CF2 and CF3. The color filter layer CFL may include a first filter CF1 that transmits the second color light, a second filter CF2 that transmits the third color light, and a third filter CF3 that transmits the first color light. For example, the first filter CF1 may be a red filter, the second filter CF2 may be a green filter, and the third filter CF3 may be a blue filter. Each of the filters CF1, CF2 and CF3 may include a polymer photosensitive resin and a pigment and/or dye. The first filter CF1 may include a red pigment and/or dye, the second filter CF2 may include a green pigment and/or dye, and the third filter CF3 may include a blue pigment and/or dye. Embodiments of the present disclosure are not limited thereto, however, and the third filter CF3 may not include the pigment or dye. The third filter CF3 may include a polymer photosensitive resin and not include a pigment or dye. The third filter CF3 may be transparent. The third filter CF3 may be formed using a transparent photosensitive resin.

In addition, in an embodiment, the first filter CF1 and the second filter CF2 may be yellow filters. The first filter CF1 and the second filter CF2 may be provided in one body without distinction. The first to third filters CF1, CF2 and CF3 may correspond to the red luminous area PXA-R, the green luminous area PXA-G and the blue luminous area PXA-B, respectively.

In some embodiments, the color filter layer CFL may further include alight blocking part. The color filter layer CFL may include a light blocking part at the boundaries to overlap with adjacent filters CF1, CF2 and CF3. The light blocking part may be a black matrix. The light blocking part may be formed by including an organic light blocking material and/or an inorganic light blocking material, including a black pigment and/or black dye. The light blocking part may divide the boundaries among adjacent filters CF1, CF2 and CF3. In addition, in an embodiment, the light blocking part may be formed as a blue filter.

A base substrate BL may be on the color filter layer CFL. The base substrate BL may be a member providing a base surface that the color filter layer CFL, the light controlling layer CCL, etc. are on. The base substrate BL may be a glass substrate, a metal substrate, a plastic substrate, etc. However, embodiments of the present disclosure are not limited thereto, and the base substrate BL may be an inorganic layer, an organic layer or a composite material layer. In some embodiments, the base substrate BL may be omitted.

FIG. 8 is a cross-sectional view showing a part of the display device according to an embodiment. In a display device DD-TD of an embodiment, the light emitting element ED-BT may include a plurality of light emitting structures OL-B1, OL-B2 and OL-B3. The light emitting element ED-BT may include a first electrode EL1 and a second electrode EL2 opposite to each other, and the plurality of light emitting structures OL-B1, OL-B2 and OL-B3 stacked in order in a thickness direction and provided between the first electrode EL1 and the second electrode EL2. Each of the light emitting structures OL-B1, OL-B2 and OL-B3 may include the emission layer EML (FIG. 7), and a hole transport region HTR and an electron transport region ETR, with the emission layer (FIG. 7) therebetween.

In some embodiments, the light emitting element ED-BT included in the display device DD-TD of an embodiment may be a light emitting element of a tandem structure including a plurality of emission layers.

In an embodiment shown in FIG. 8, light emitted from the light emitting structures OL-B1, OL-B2 and OL-B3 may be all blue light. However, embodiments of the present disclosure are not limited thereto, and the wavelength regions of light emitted from the light emitting structures OL-B1, OL-B2 and OL-B3 may be different from each other. For example, the light emitting element ED-BT including the plurality of light emitting structures OL-B1, OL-B2 and OL-B3 that emit light in different wavelength regions may emit white light.

Charge generating layers CGL1 and CGL2 may be between neighboring light emitting structures OL-B1, OL-B2 and OL-B3. The charge generating layers CGL1 and CGL2 may include a p-type charge generating layer and/or an n-type charge generating layer.

In at least one selected from among the light emitting structures OL-B1, OL-B2 and OL-B3, included in the display device DD-TD of an embodiment, the amine compound of an embodiment may be included.

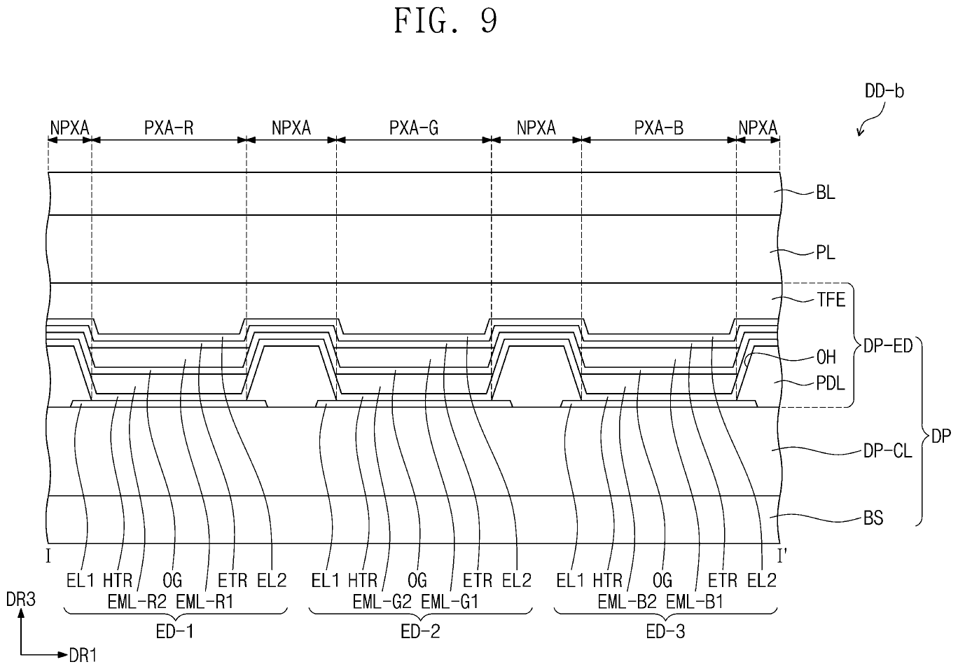

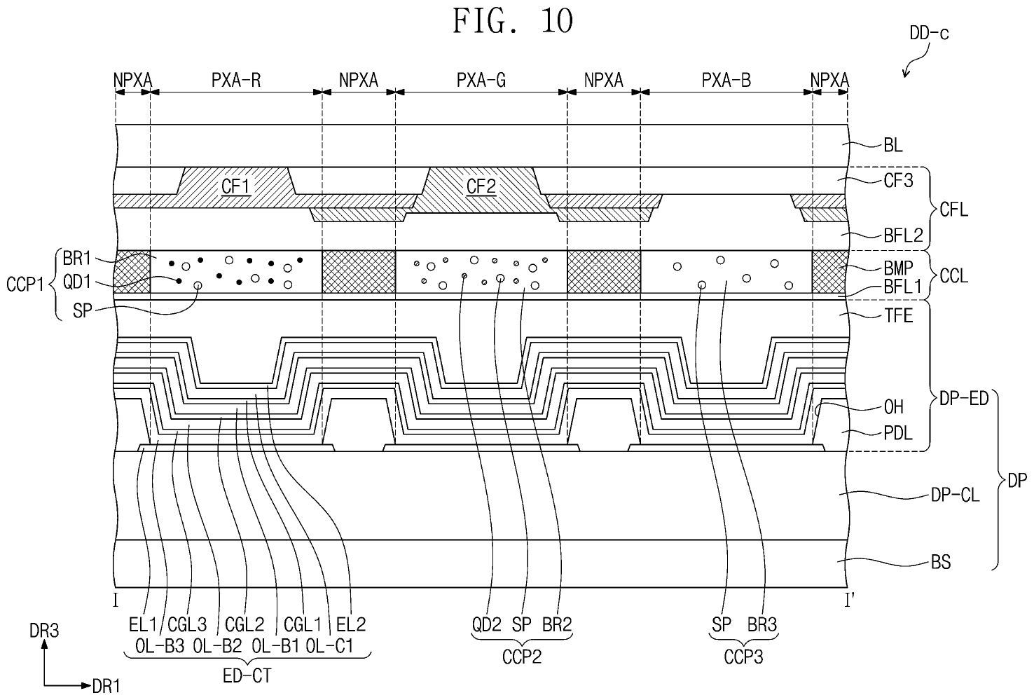

Referring to FIG. 9, a display device DD-b according to an embodiment may include light emitting elements ED-1, ED-2 and ED-3, formed by stacking two emission layers. Compared to the display device DD of an embodiment, shown in FIG. 2, an embodiment shown in FIG. 9 is different in that first to third light emitting elements ED-1, ED-2 and ED-3 include two emission layers stacked in a thickness direction, each. In the first to third light emitting elements ED-1, ED-2 and ED-3, two emission layers may emit light in the same wavelength region.

The first light emitting element ED-1 may include a first red emission layer EML-R1 and a second red emission layer EML-R2. The second light emitting element ED-2 may include a first green emission layer EML-G1 and a second green emission layer EML-G2. In addition, the third light emitting element ED-3 may include a first blue emission layer EML-B1 and a second blue emission layer EML-B2. An emission auxiliary part OG may be between the first red emission layer EML-R1 and the second red emission layer EML-R2, between the first green emission layer EML-G1 and the second green emission layer EML-G2, and between the first blue emission layer EML-B1 and the second blue emission layer EML-B2.

The emission auxiliary part OG may include a single layer or a multilayer. The emission auxiliary part OG may include a charge generating layer. In some embodiments, the emission auxiliary part OG may include an electron transport region, a charge generating layer, and a hole transport region stacked in order. The emission auxiliary part OG may be provided as a common layer in all of the first to third light emitting elements ED-1, ED-2 and ED-3. However, embodiments of the present disclosure are not limited thereto, and the emission auxiliary part OG may be patterned and provided in an opening part OH defined in a pixel definition layer PDL.

The first red emission layer EML-R1, the first green emission layer EML-G1 and the first blue emission layer EML-B1 may be between the hole transport region HTR and the emission auxiliary part OG. The second red emission layer EML-R2, the second green emission layer EML-G2 and the second blue emission layer EML-B2 may be between the emission auxiliary part OG and the electron transport region ETR.

In some embodiments, the first light emitting element ED-1 may include a first electrode EL1, a hole transport region HTR, a second red emission layer EML-R2, an emission auxiliary part OG, a first red emission layer EML-R1, an electron transport region ETR, and a second electrode EL2, stacked in order. The second light emitting element ED-2 may include a first electrode EL1, a hole transport region HTR, a second green emission layer EML-G2, an emission auxiliary part OG, a first green emission layer EML-G1, an electron transport region ETR, and a second electrode EL2, stacked in order. The third light emitting element ED-3 may include a first electrode EL1, a hole transport region HTR, a second blue emission layer EML-B2, an emission auxiliary part OG, a first blue emission layer EML-B1, an electron transport region ETR, and a second electrode EL2, stacked in order.

In some embodiments, an optical auxiliary layer PL may be on a display element layer DP-ED. The optical auxiliary layer PL may include a polarization layer. The optical auxiliary layer PL may be on a display panel DP and may control reflected light at the display panel DP by external light. In some embodiments, the optical auxiliary layer PL may be omitted from the display device.

Different from FIG. 8 and FIG. 9, a display device DD-c in FIG. 10 is shown to include four light emitting structures OL-B1, OL-B2, OL-B3 and OL-C1. A light emitting element ED-CT may include a first electrode EL1 and a second electrode EL2 opposite to each other, and first to fourth light emitting structures OL-B1, OL-B2, OL-B3 and OL-C1 stacked in order in a thickness direction between the first electrode EL1 and the second electrode EL2. Charge generating layers CGL1, CGL2 and CGL3 may be between the first to fourth light emitting structures OL-B1, OL-B2, OL-B3 and OL-C1. Among the four light emitting structures, the first to third light emitting structures OL-B1, OL-B2 and OL-B3 may emit blue light, and the fourth light emitting structure OL-C1 may emit green light. However, embodiments of the present disclosure are not limited thereto, and the first to fourth light emitting structures OL-B1, OL-B2, OL-B3 and OL-C1 may emit different wavelengths of light.

Charge generating layers CGL1, CGL2 and CGL3 located among neighboring light emitting structures OL-B1, OL-B2, OL-B3 and OL-C1 may include a p-type charge generating layer and/or an n-type charge generating layer.

In at least one selected from among the light emitting structures OL-B1, OL-B2, OL-B3 and OL-C1, included in the display device DD-c of an embodiment, the amine compound of an embodiment may be included.

The light emitting element ED according to an embodiment of the present disclosure may include the amine compound of an embodiment in at least one functional layer between the first electrode EL1 and the second electrode EL2 to show improved emission efficiency and improved life characteristics. The light emitting element ED according to an embodiment may include the amine compound of an embodiment in at least one selected from among a hole transport region HTR, an emission layer EML, and an electron transport region ETR, between the first electrode EL1 and the second electrode EL2, and/or in a capping layer CPL. For example, the amine compound according to an embodiment may be included in the hole transport region HTR of the light emitting element ED of an embodiment, and the light emitting element of an embodiment may show high efficiency and long-life characteristics.

The amine compound of an embodiment includes first, second and third substituents and may improve the stability of a material and improve hole transport properties. Accordingly, the lifetime and efficiency of the light emitting element including the amine compound of an embodiment may be improved. In addition, the light emitting element of an embodiment may include the amine compound according to an embodiment in a hole transport layer to show improved efficiency and lifetime characteristics.

Hereinafter, referring to embodiments and comparative embodiments, the amine compound according to an embodiment and the light emitting element according to an embodiment of the present disclosure will be explained in more detail herein below. In addition, the embodiments below are illustrations to assist the understanding of the subject matter of the present disclosure, but the scope of the present disclosure is not limited thereto.

### EXAMPLES

### 1. Synthesis of Amine Compounds

First, the synthetic methods of the amine compounds according to embodiments will be explained in more detail by illustrating the synthetic methods for synthesizing Compound 1, Compound 6, Compound 21, Compound 26, Compound 41, Compound 46, Compound 61, Compound 66, Compound 81, Compound 86, Compound 101, Compound 106, Compound 121, Compound 126, Compound 141, Compound 146, Compound 161, Compound 166, Compound 181, Compound 186, Compound 201, Compound 206, Compound 221, and Compound 226, shown in Table 1. In addition, the synthetic methods of the amine compounds explained hereinafter are embodiments, and the synthetic method of the amine compound according to embodiments of the present disclosure is not limited to the embodiments below.

### Synthetic Method of Compounds

### (1) Synthesis of Compound 1

Compound 1 according to an embodiment may be synthesized by, for example, the reactions below.

### Synthesis of Intermediate 1-1

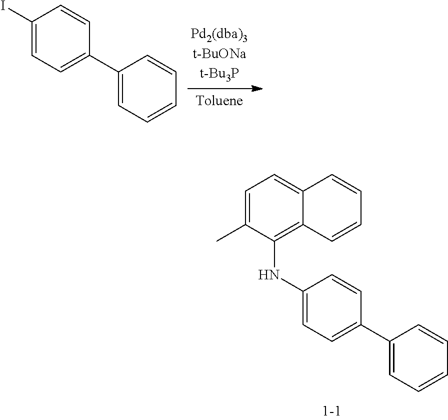 [SMILES: Ic1ccc(-c2ccccc2)cc1 ; Cc1ccc2ccccc2c1Nc1ccc(-c2ccccc2)cc1]

4-Iodo-1,1′-biphenyl (100 mmol, 1 eq), 2-methylnaphthalen-1-amine (130 mmol, 1.3 eq), Pd₂(dba)₃ (3 mmol, 0.03 eq), t-BuONa (200 mmol, 2 eq), t-Bu₃P (6 mmol, 0.06 eq), and 500 ml of toluene were added to a 1-neck-round flask, followed by stirring at about 90° C. for about 3 hours. After finishing the reaction, the resultant reaction product was worked up with ether/H₂O, and then separated by column chromatography using methylene chloride/hexane=1/7 to obtain Intermediate 1-1 (76 mmol, yield=76%).

### Synthesis of Compound 1

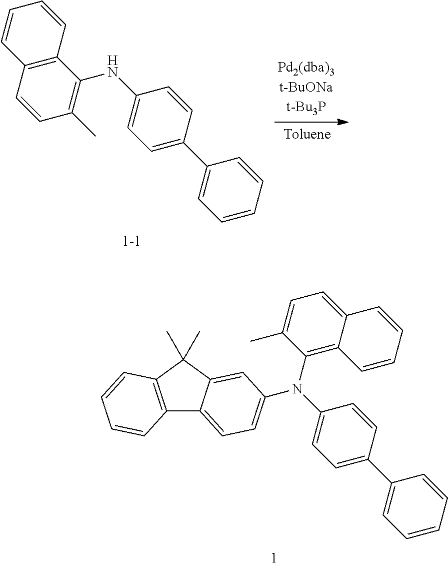 [SMILES: Cc1ccc2ccccc2c1Nc1ccc(-c2ccccc2)cc1 ; Cc1ccc2ccccc2c1N(c1ccc(-c2ccccc2)cc1)c1ccc2c(c1)C(C)(C)c1ccccc1-2]

Intermediate 1-1 (11 mmol, 1.1 eq), 2-bromo-9,9-dimethyl-9H-fluorene (10 mmol, 1 eq), Pd₂(dba)₃ (0.5 mmol, 0.05 eq), t-BuONa (20 mmol, 2 eq), t-Bu₃P (1 mmol, 0.1 eq), and 150 ml of toluene were added to a 1-neck-round flask, followed by stirring at about 110° C. for about 1 hour. After finishing the reaction, the resultant reaction product was worked up with ether/H₂O, and then separated by column chromatography using methylene chloride/hexane=1/7 to obtain Compound 1 (7.6 mmol, Y=76%).

### (2) Synthesis of Compound 6

Compound 6 according to an embodiment may be synthesized by, for example, the reactions below.

### Synthesis of Intermediate 6-1

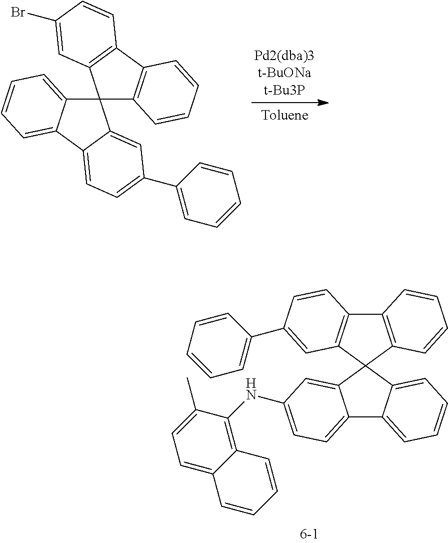 [SMILES: Brc1ccc2c(c1)C1(c3ccccc3-2)c2ccccc2-c2ccc(-c3ccccc3)cc21 ; Cc1ccc2ccccc2c1Nc1ccc2c(c1)C1(c3ccccc3-2)c2ccccc2-c2ccc(-c3ccccc3)cc21]

2-Bromo-2′-phenyl-9,9′-spirobi[fluorene] (100 mmol, 1 eq), 2-methylnaphthalen-1-amine (130 mmol, 1.3 eq), Pd₂(dba)₃ (3 mmol, 0.03 eq), t-BuONa (200 mmol, 2 eq), t-Bu₃P (6 mmol, 0.06 eq), and 500 ml of toluene were added to a 1-neck-round flask, followed by stirring at about 90° C. for about 3 hours. After finishing the reaction, the resultant reaction product was worked up with ether/H₂O, and then separated by column chromatography using methylene chloride/hexane=1/7 to obtain Intermediate 6-1 (8.8 mmol, Y=88%).

### Synthesis of Compound 6

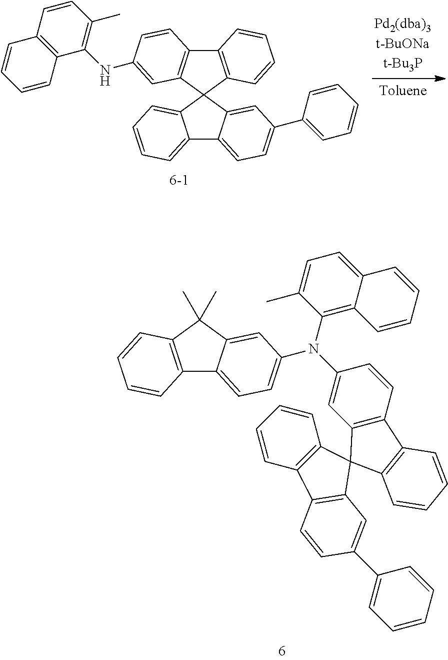 [SMILES: Cc1ccc2ccccc2c1Nc1ccc2c(c1)C1(c3ccccc3-2)c2ccccc2-c2ccc(-c3ccccc3)cc21 ; Cc1ccc2ccccc2c1N(c1ccc2c(c1)C(C)(C)c1ccccc1-2)c1ccc2c(c1)C1(c3ccccc3-c3ccc(-c4ccccc4)cc31)c1ccccc1-2]

Intermediate 6-1 (11 mmol, 1.1 eq), 2-bromo-9,9-dimethyl-9H-fluorene (10 mmol, 1 eq), Pd₂(dba)₃ (0.5 mmol, 0.05 eq), t-BuONa (20 mmol, 2 eq), t-Bu₃P (1 mmol, 0.1 eq), and 150 ml of toluene were added to a 1-neck-round flask, followed by stirring at about 110° C. for about 1 hour.

After finishing the reaction, the resultant reaction product was worked up with ether/H₂O, and then separated by column chromatography using methylene chloride/hexane=1/7 to obtain Compound 6 (7 mmol, Y=70%).

### (3) Synthesis of Compound 21

Compound 21 according to an embodiment may be synthesized by, for example, the reaction below.

### Synthesis of Compound

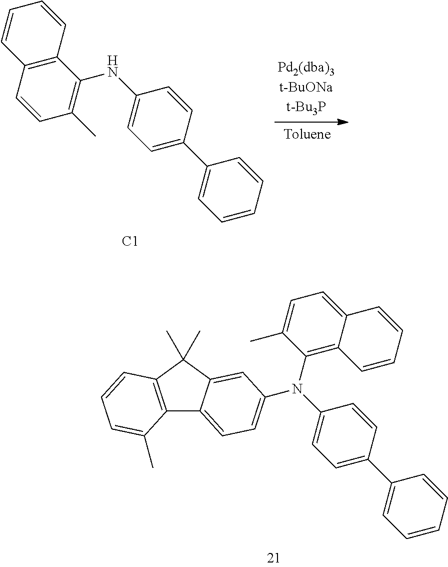 [SMILES: Cc1ccc2ccccc2c1Nc1ccc(-c2ccccc2)cc1 ; "C1" ; Cc1cccc2c1-c1ccc(N(c3ccc(-c4ccccc4)cc3)c3c(C)ccc4ccccc34)cc1C2(C)C]

Intermediate 1-1 (11 mmol, 1.1 eq), 2-bromo-5,9,9-trimethyl-9H-fluorene (10 mmol, 1 eq), Pd₂(dba)₃ (0.5 mmol, 0.05 eq), t-BuONa (20 mmol, 2 eq), t-Bu₃P (1 mmol, 0.1 eq), and 150 ml of toluene were added to a 1-neck-round flask, followed by stirring at about 110° C. for about 1 hour.

After finishing the reaction, the resultant reaction product was worked up with ether/H₂O, and then separated by column chromatography using methylene chloride/hexane=1/7 to obtain Compound 21 (8.3 mmol, Y=83%).

### (4) Synthesis of Compound 26

Compound 26 according to an embodiment may be synthesized by, for example, the reaction below.

### Synthesis of Compound 26

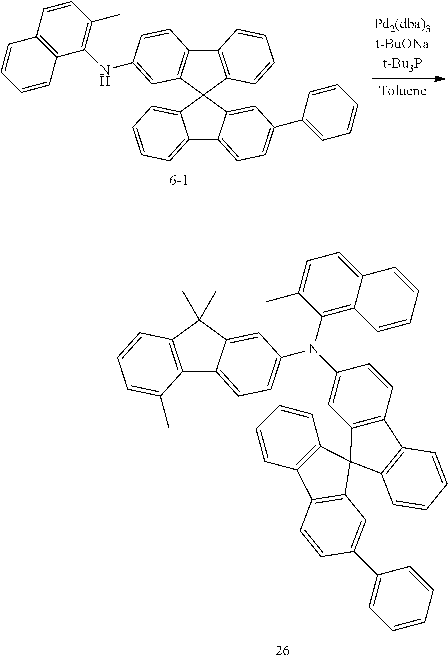 [SMILES: Cc1ccc2ccccc2c1Nc1ccc2c(c1)C1(c3ccccc3-2)c2ccccc2-c2ccc(-c3ccccc3)cc21 ; Cc1cccc2c1-c1ccc(N(c3ccc4c(c3)C3(c5ccccc5-c5ccc(-c6ccccc6)cc53)c3ccccc3-4)c3c(C)ccc4ccccc34)cc1C2(C)C]

Intermediate 6-1 (11 mmol, 1.1 eq), 2-bromo-5,9,9-trimethyl-9H-fluorene (10 mmol, 1 eq), Pd₂(dba)₃ (0.5 mmol, 0.05 eq), t-BuONa (20 mmol, 2 eq), t-Bu₃P (1 mmol, 0.1 eq), and 150 ml of toluene were added to a 1-neck-round flask, followed by stirring at about 110° C. for about 1 hour.

After finishing the reaction, the resultant reaction product was worked up with ether/H₂O, and then separated by column chromatography using methylene chloride/hexane=1/7 to obtain Compound 26 (8 mmol, Y=80%).

### (5) Synthesis of Compound 41

Compound 41 according to an embodiment may be synthesized by, for example, the reaction below.

### Synthesis of Compound 41

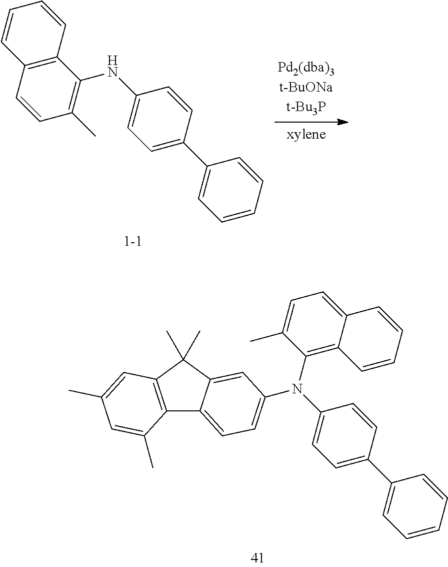 [SMILES: Cc1ccc2ccccc2c1Nc1ccc(-c2ccccc2)cc1 ; "1-1" ; Cc1cc(C)c2c(c1)C(C)(C)c1cc(N(c3ccc(-c4ccccc4)cc3)c3c(C)ccc4ccccc34)ccc1-2]

Intermediate 1-1 (11 mmol, 1.1 eq), 7-chloro-2,4,9,9-tetramethyl-9H-fluorene (10 mmol, 1 eq), Pd₂(dba)₃ (0.5 mmol, 0.05 eq), t-BuONa (20 mmol, 2 eq), t-Bu₃P (1 mmol, 0.1 eq), and 150 ml of xylene were added to a 1-neck-round flask, followed by stirring at about 150° C. for about 3 hours.

After finishing the reaction, the resultant reaction product was worked up with ether/H₂O, and then separated by column chromatography using methylene chloride/hexane=1/6 to obtain Compound 41 (8.1 mmol, Y=81%).

### (6) Synthesis of Compound 46

Compound 46 according to an embodiment may be synthesized by, for example, the reaction below.

### Synthesis of Compound 46

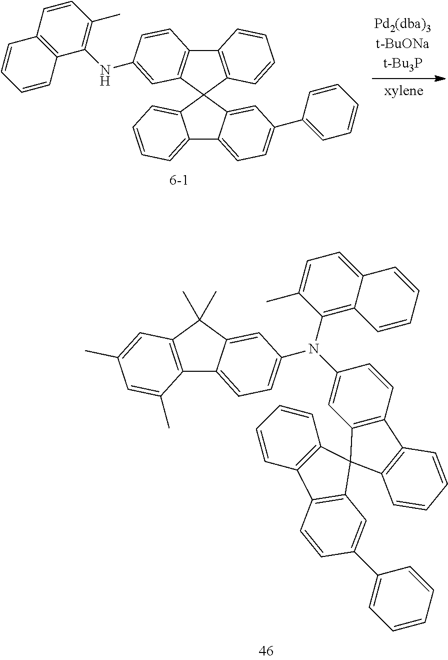 [SMILES: Cc1ccc2ccccc2c1Nc1ccc2c(c1)C1(c3ccccc3-2)c2ccccc2-c2ccc(-c3ccccc3)cc21 ; Cc1cc(C)c2c(c1)C(C)(C)c1cc(N(c3ccc4c(c3)C3(c5ccccc5-c5ccc(-c6ccccc6)cc53)c3ccccc3-4)c3c(C)ccc4ccccc34)ccc1-2]

Intermediate 6-1 (11 mmol, 1.1 eq), 7-chloro-2,4,9,9-tetramethyl-9H-fluorene (10 mmol, 1 eq), Pd₂(dba)₃ (0.5 mmol, 0.05 eq), t-BuONa (20 mmol, 2 eq), t-Bu₃P (1 mmol, 0.1 eq), and 150 ml of xylene were added to a 1-neck-round flask, followed by stirring at about 150° C. for about 3 hours.

After finishing the reaction, the resultant reaction product was worked up with ether/H₂O, and then separated by column chromatography using methylene chloride/hexane=1/6 to obtain Compound 46 (8.4 mmol, Y=84%).

### (7) Synthesis of Compound 61

Compound 61 according to an embodiment may be synthesized by, for example, the reaction below.

### Synthesis of Compound 61

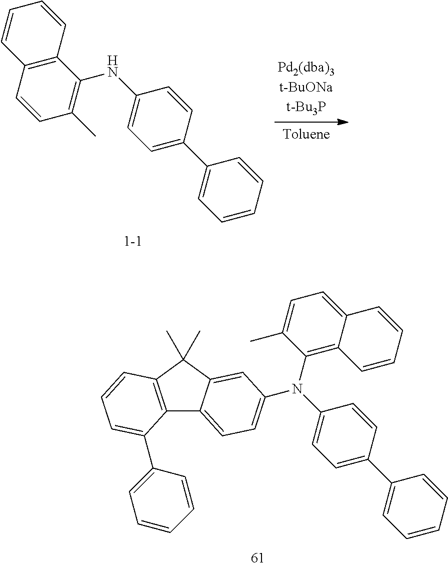 [SMILES: Cc1ccc2ccccc2c1Nc1ccc(-c2ccccc2)cc1 ; "1-1" ; Cc1ccc2ccccc2c1N(c1ccc(-c2ccccc2)cc1)c1ccc2c(c1)C(C)(C)c1cccc(-c3ccccc3)c1-2]

Intermediate 1-1 (11 mmol, 1.1 eq), 2-bromo-9,9-dimethyl-5-phenyl-9H-fluorene (10 mmol, 1 eq), Pd₂(dba)₃ (0.5 mmol, 0.05 eq), t-BuONa (20 mmol, 2 eq), t-Bu³P (1 mmol, 0.1 eq), and 150 ml of toluene were added to a 1-neck-round flask, followed by stirring at about 110° C. for about 1 hour.

After finishing the reaction, the resultant reaction product was worked up with ether/H₂O, and then separated by column chromatography using methylene chloride/hexane=1/7 to obtain Compound 61 (8.5 mmol, Y=85%).

### (8) Synthesis of Compound 66

Compound 66 according to an embodiment may be synthesized by, for example, the reaction below.

### Synthesis of Compound 66

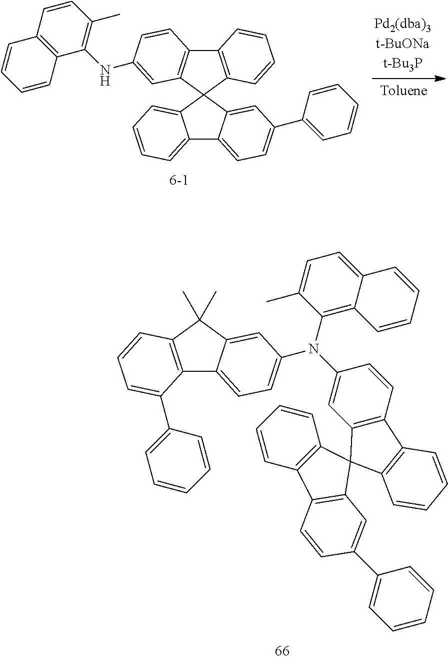 [SMILES: Cc1ccc2ccccc2c1Nc1ccc2c(c1)C1(c3ccccc3-2)c2ccccc2-c2ccc(-c3ccccc3)cc21 ; Cc1ccc2ccccc2c1N(c1ccc2c(c1)C(C)(C)c1cccc(-c3ccccc3)c1-2)c1ccc2c(c1)C1(c3ccccc3-c3ccc(-c4ccccc4)cc31)c1ccccc1-2]

Intermediate 6-1 (11 mmol, 1.1 eq), 2-bromo-9,9-dimethyl-5-phenyl-9H-fluorene (10 mmol, 1 eq), Pd₂(dba)₃ (0.5 mmol, 0.05 eq), t-BuONa (20 mmol, 2 eq), t-BusP (1 mmol, 0.1 eq), and 150 ml of toluene were added to a 1-neck-round flask, followed by stirring at about 110° C. for about 1 hour.

After finishing the reaction, the resultant reaction product was worked up with ether/H₂O, and then separated by column chromatography using methylene chloride/hexane=1/7 to obtain Compound 66 (8 mmol, Y=80%).

### (9) Synthesis of Compound 81

Compound 81 according to an embodiment may be synthesized by, for example, the reactions below.

### Synthesis of Intermediate 8-11

The same process was performed as the synthesis of Intermediate 1-1, except for using 3-methynaphthalen-2-amine instead of 2-methyinaphthalen-1-amine.

### Synthesis of Compound 81

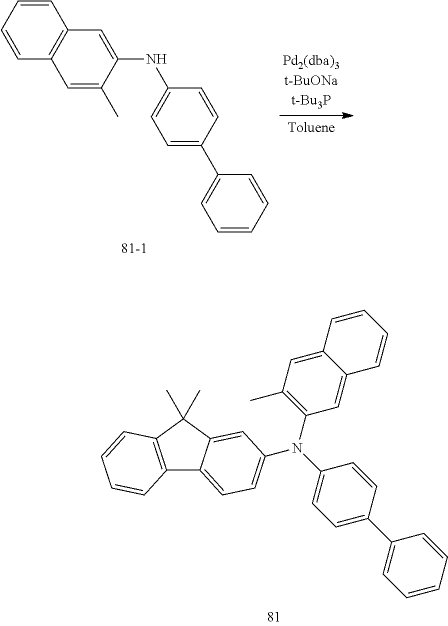 [SMILES: Cc1cc2ccccc2cc1Nc1ccc(-c2ccccc2)cc1 ; "81-1" ; Cc1cc2ccccc2cc1N(c1ccc(-c2ccccc2)cc1)c1ccc2c(c1)C(C)(C)c1ccccc1-2]

The same process was performed as the synthesis of Compound 1 except for using Intermediate 81-1 instead of Intermediate 1-1 to obtain Compound 81 (6.8 mmol, Y=68%).

### (10) Synthesis of Compound 86

Compound 86 according to an embodiment may be synthesized by, for example, the reactions below.

### Synthesis of Intermediate 86-1

The same process was performed as the synthesis of Intermediate 6-1, except for using 3-methylnaphthalen-2-amine instead of 2-methylnaphthalen-1-amine.

### Synthesis of Compound 86

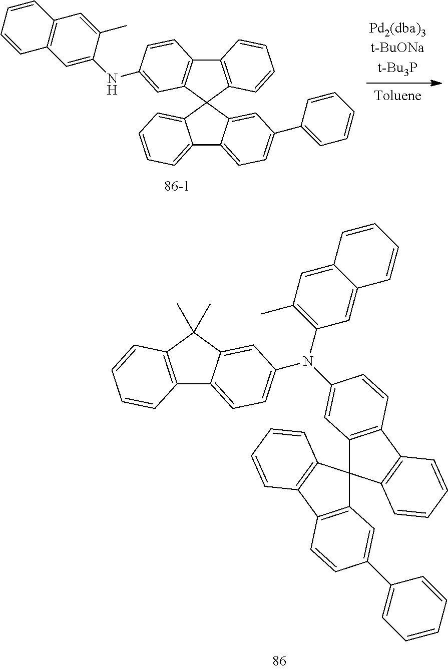 [SMILES: Cc1cc2ccccc2cc1Nc1ccc2c(c1)C1(c3ccccc3-2)c2ccccc2-c2ccc(-c3ccccc3)cc21 ; Cc1cc2ccccc2cc1N(c1ccc2c(c1)C(C)(C)c1ccccc1-2)c1ccc2c(c1)C1(c3ccccc3-c3ccc(-c4ccccc4)cc31)c1ccccc1-2]

The same process was performed as the synthesis of Compound 6 except for using Intermediate 86-1 instead of Intermediate 6-1 to obtain Compound 86 (7.2 mmol, Y=72%).

### (11) Synthesis of Compound 101

Compound 101 according to an embodiment may be synthesized by, for example, the reaction below.

### Synthesis of Compound 101

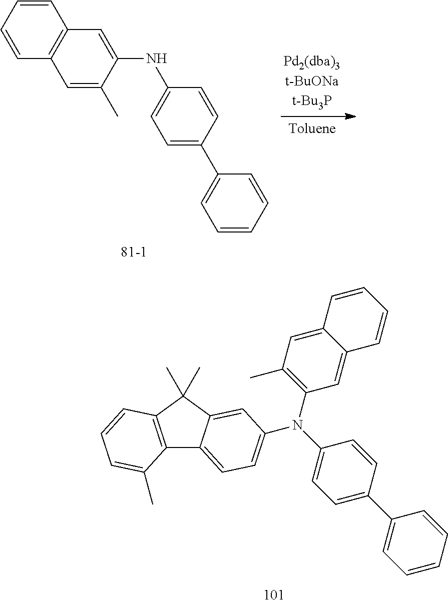 [SMILES: Cc1cc2ccccc2cc1Nc1ccc(-c2ccccc2)cc1 ; Cc1cc2ccccc2cc1N(c1ccc(-c2ccccc2)cc1)c1ccc2c(c1)C(C)(C)c1cccc(C)c1-2]

The same process was performed as the synthesis of Compound 21 except for using Intermediate 81-1 instead of Intermediate 1-1 to obtain Compound 101 (8.3 mmol, Y=83%).

### (12) Synthesis of Compound 106

Compound 106 according to an embodiment may be synthesized by, for example, the reaction below.

### Synthesis of Compound 106

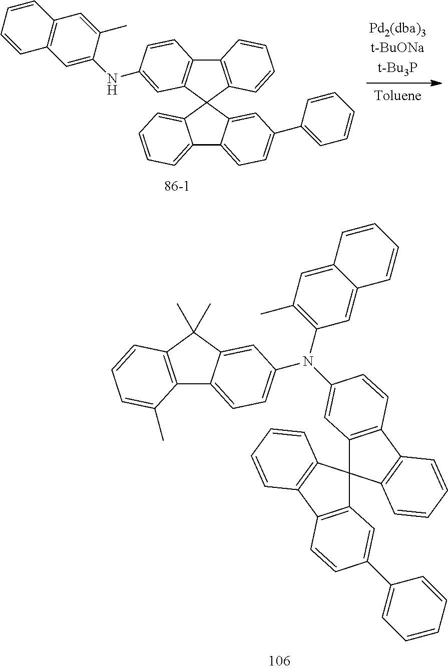 [SMILES: Cc1cc2ccccc2cc1Nc1ccc2c(c1)C1(c3ccccc3-2)c2ccccc2-c2ccc(-c3ccccc3)cc21 ; Cc1cc2ccccc2cc1N(c1ccc2c(c1)C(C)(C)c1cccc(C)c1-2)c1ccc2c(c1)C1(c3ccccc3-c3ccc(-c4ccccc4)cc31)c1ccccc1-2]

The same process was performed as the synthesis of Compound 26 except for using Intermediate 86-1 instead of Intermediate 6-1 to obtain Compound 106 (8.1 mmol, Y=81%).

### (13) Synthesis of Compound 121

Compound 121 according to an embodiment may be synthesized by, for example, the reaction below.

### Synthesis of Compound 121

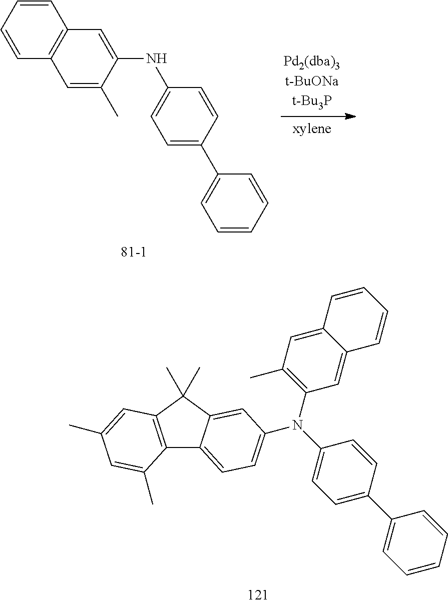 [SMILES: Cc1cc2ccccc2cc1Nc1ccc(-c2ccccc2)cc1 ; Cc1cc(C)c2c(c1)C(C)(C)c1cc(N(c3ccc(-c4ccccc4)cc3)c3cc4ccccc4cc3C)ccc1-2]

The same process was performed as the synthesis of Compound 41 except for using Intermediate 81-1 instead of Intermediate 1-1 to obtain Compound 121 (8.5 mmol, Y=85%).

### (14) Synthesis of Compound 126

Compound 126 according to an embodiment may be synthesized by, for example, the reaction below.

### Synthesis of Compound 126

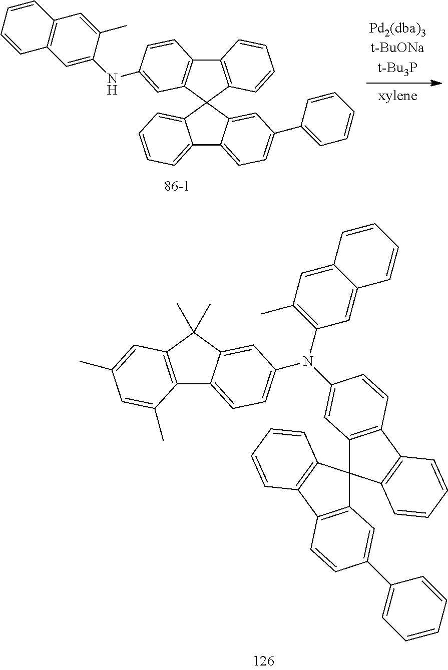 [SMILES: Cc1cc2ccccc2cc1Nc1ccc2c(c1)C1(c3ccccc3-2)c2ccccc2-c2ccc(-c3ccccc3)cc21 ; Cc1cc(C)c2c(c1)C(C)(C)c1cc(N(c3ccc4c(c3)C3(c5ccccc5-c5ccc(-c6ccccc6)cc53)c3ccccc3-4)c3cc4ccccc4cc3C)ccc1-2]

The same process was performed as the synthesis of Compound 46 except for using Intermediate 86-1 instead of Intermediate 6-1 to obtain Compound 126 (8.0 mmol, Y=80%).

### (15) Synthesis of Compound 141

Compound 141 according to an embodiment may be synthesized by, for example, the reaction below.

### Synthesis of Compound 141

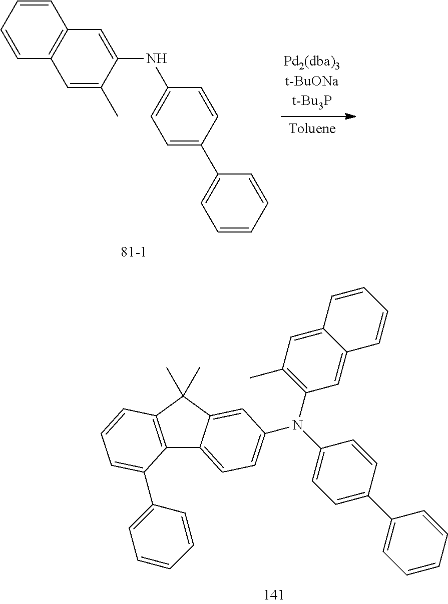 [SMILES: Cc1cc2ccccc2cc1Nc1ccc(-c2ccccc2)cc1 ; Cc1cc2ccccc2cc1N(c1ccc(-c2ccccc2)cc1)c1ccc2c(c1)C(C)(C)c1cccc(-c3ccccc3)c1-2]

The same process was performed as the synthesis of Compound 61 except for using Intermediate 81-1 instead of Intermediate 1-1 to obtain Compound 141 (8.8 mmol, Y=88%).

### (16) Synthesis of Compound 146

Compound 146 according to an embodiment may be synthesized by, for example, the reaction below.

### Synthesis of Compound 146

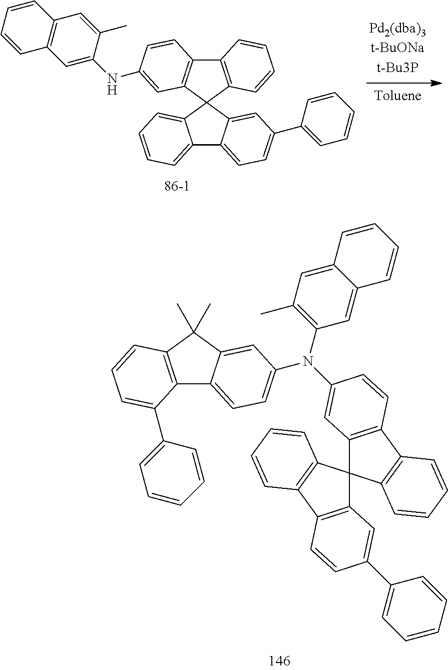 [SMILES: Cc1cc2ccccc2cc1Nc1ccc2c(c1)C1(c3ccccc3-2)c2ccccc2-c2ccc(-c3ccccc3)cc21 ; Cc1cc2ccccc2cc1N(c1ccc2c(c1)C(C)(C)c1cccc(-c3ccccc3)c1-2)c1ccc2c(c1)C1(c3ccccc3-c3ccc(-c4ccccc4)cc31)c1ccccc1-2]

The same process was performed as the synthesis of Compound 66 except for using Intermediate 86-1 instead of Intermediate 6-1 to obtain Compound 146 (8.1 mmol, Y=81%).

### (17) Synthesis of Compound 161

Compound 161 according to an embodiment may be synthesized by, for example, the reactions below.

### Synthesis of Intermediate 161-1

The same process was performed as the synthesis of Intermediate 1-1 except for using 1-methylnaphthalen-2-amine instead of 2-methylnaphthalen-1-amine.

### Synthesis of Compound 161

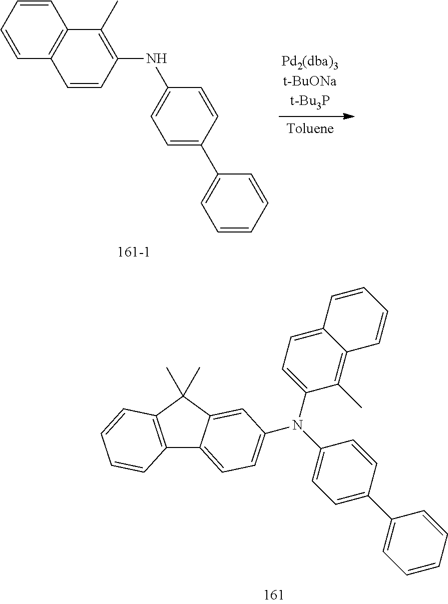 [SMILES: Cc1c(Nc2ccc(-c3ccccc3)cc2)ccc2ccccc12 ; Cc1c(N(c2ccc(-c3ccccc3)cc2)c2ccc3c(c2)C(C)(C)c2ccccc2-3)ccc2ccccc12]

The same process was performed as the synthesis of Compound 1 except for using Intermediate 161-1 instead of Intermediate 1-1 to obtain Compound 161 (7.1 mmol, Y=71%).

### (18) Synthesis of Compound 166

Compound 166 according to an embodiment may be synthesized by, for example, the reactions below.

### Synthesis of Intermediate 166-1

The same process was performed as the synthesis of Intermediate 6-1, except for using 1-methylnaphthalen-2-amine instead of 2-methylnaphthalen-1-amine.

### Synthesis of Compound 166

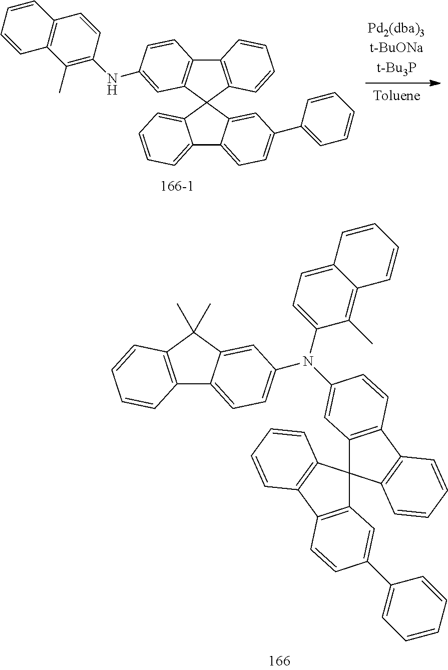 [SMILES: Cc1c(Nc2ccc3c(c2)C2(c4ccccc4-3)c3ccccc3-c3ccc(-c4ccccc4)cc32)ccc2ccccc12 ; Cc1c(N(c2ccc3c(c2)C(C)(C)c2ccccc2-3)c2ccc3c(c2)C2(c4ccccc4-c4ccc(-c5ccccc5)cc42)c2ccccc2-3)ccc2ccccc12]

The same process was performed as the synthesis of Compound 6 except for using Intermediate 166-1 instead of Intermediate 6-1 to obtain Compound 166 (7.2 mmol, Y=72%).

### (19) Synthesis of Compound 181

Compound 181 according to an embodiment may be synthesized by, for example, the reaction below.

### Synthesis of Compound 181

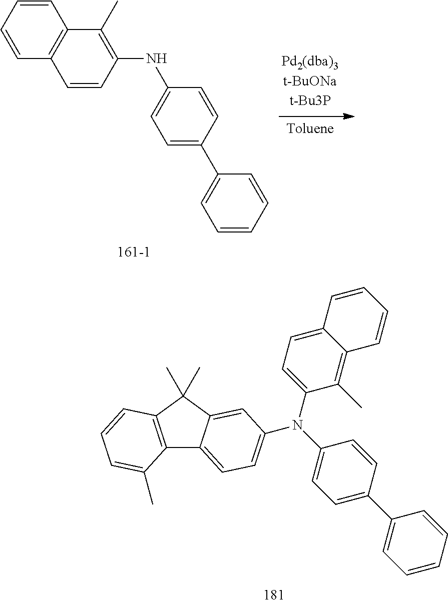 [SMILES: Cc1c(Nc2ccc(-c3ccccc3)cc2)ccc2ccccc12 ; Cc1cccc2c1-c1ccc(N(c3ccc(-c4ccccc4)cc3)c3ccc4ccccc4c3C)cc1C2(C)C]

The same process was performed as the synthesis of Compound 21 except for using Intermediate 161-1 instead of Intermediate 1-1 to obtain Compound 181 (8.1 mmol, Y=81%).

### (20) Synthesis of Compound 186

Compound 186 according to an embodiment may be synthesized by, for example, the reaction below.

### Synthesis of Compound 186

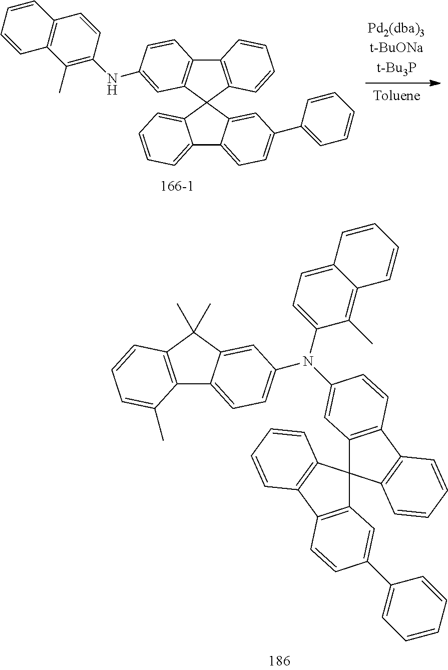 [SMILES: Cc1c(Nc2ccc3c(c2)C2(c4ccccc4-3)c3ccccc3-c3ccc(-c4ccccc4)cc32)ccc2ccccc12 ; Cc1cccc2c1-c1ccc(N(c3ccc4c(c3)C3(c5ccccc5-c5ccc(-c6ccccc6)cc53)c3ccccc3-4)c3ccc4ccccc4c3C)cc1C2(C)C]

The same process was performed as the synthesis of Compound 26 except for using Intermediate 166-1 instead of Intermediate 6-1 to obtain Compound 186 (7.8 mmol, Y=78%).

### (21) Synthesis of Compound 201

Compound 201 according to an embodiment may be synthesized by, for example, the reaction below.

### Synthesis of Compound 201

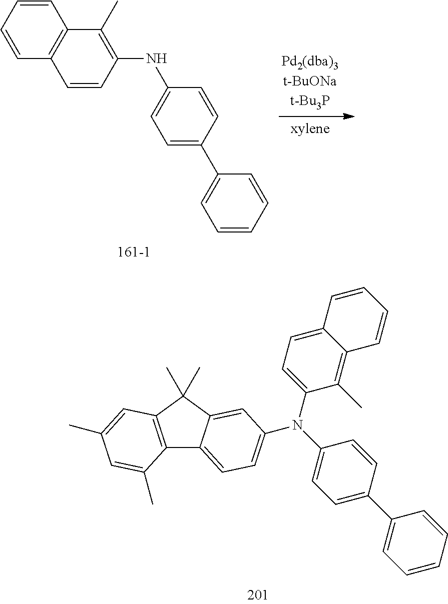 [SMILES: Cc1c(Nc2ccc(-c3ccccc3)cc2)ccc2ccccc12 ; Cc1cc(C)c2c(c1)C(C)(C)c1cc(N(c3ccc(-c4ccccc4)cc3)c3ccc4ccccc4c3C)ccc1-2]

The same process was performed as the synthesis of Compound 41 except for using Intermediate 161-1 instead of Intermediate 1-1 to obtain Compound 201 (8.0 mmol, Y=80%).

### (22) Synthesis of Compound 206

Compound 206 according to an embodiment may be synthesized by, for example, the reaction below.

### Synthesis of Compound 206

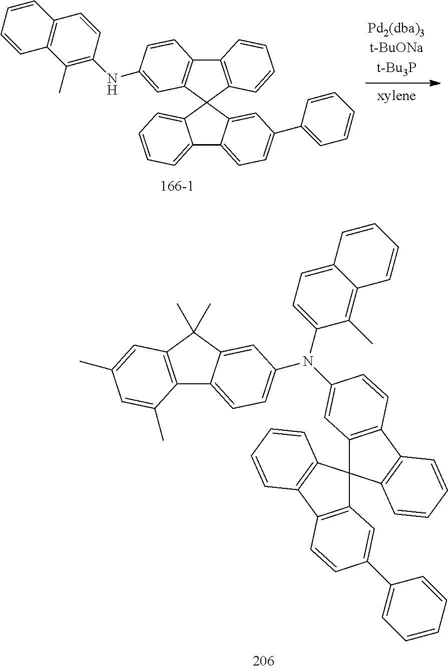 [SMILES: Cc1c(Nc2ccc3c(c2)C2(c4ccccc4-3)c3ccccc3-c3ccc(-c4ccccc4)cc32)ccc2ccccc12 ; Cc1cc(C)c2c(c1)C(C)(C)c1cc(N(c3ccc4c(c3)C3(c5ccccc5-c5ccc(-c6ccccc6)cc53)c3ccccc3-4)c3ccc4ccccc4c3C)ccc1-2]

The same process was performed as the synthesis of Compound 46 except for using Intermediate 166-1 instead of Intermediate 6-1 to obtain Compound 206 (7.7 mmol, Y=77%).

### (23) Synthesis of Compound 221

Compound 221 according to an embodiment may be synthesized by, for example, the reaction below.

### Synthesis of Compound 221

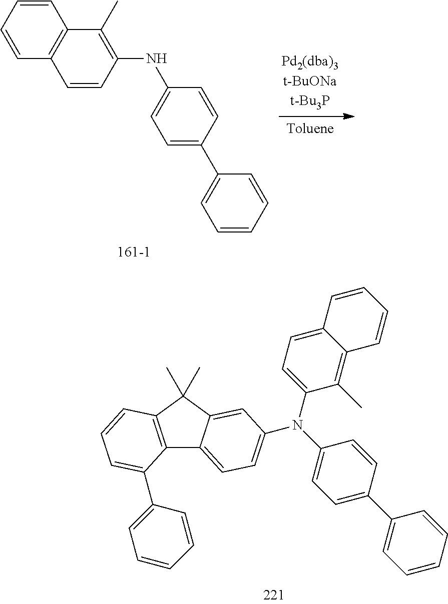 [SMILES: Cc1c(Nc2ccc(-c3ccccc3)cc2)ccc2ccccc12 ; Cc1c(N(c2ccc(-c3ccccc3)cc2)c2ccc3c(c2)C(C)(C)c2cccc(-c4ccccc4)c2-3)ccc2ccccc12]

The same process was performed as the synthesis of Compound 61 except for using Intermediate 161-1 instead of Intermediate 1-1 to obtain Compound 221 (8.1 mmol, Y=81%).

### (24) Synthesis of Compound 226

Compound 226 according to an embodiment may be synthesized by, for example, the reaction below.

### Synthesis of Compound 226

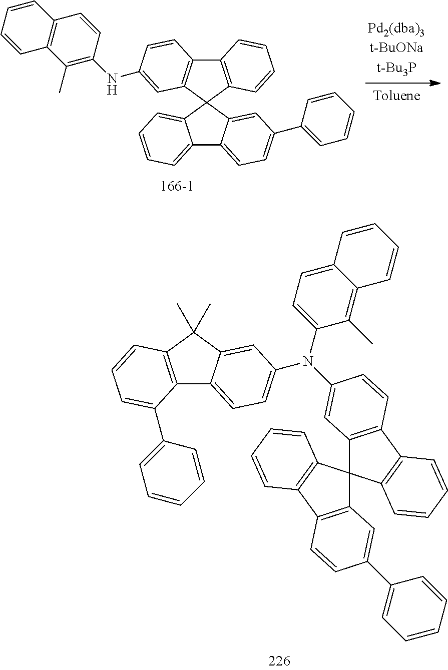 [SMILES: Cc1c(Nc2ccc3c(c2)C2(c4ccccc4-3)c3ccccc3-c3ccc(-c4ccccc4)cc32)ccc2ccccc12 ; Cc1c(N(c2ccc3c(c2)C(C)(C)c2cccc(-c4ccccc4)c2-3)c2ccc3c(c2)C2(c4ccccc4-c4ccc(-c5ccccc5)cc42)c2ccccc2-3)ccc2ccccc12]

The same process was performed as the synthesis of Compound 66 except for using Intermediate 166-1 instead of Intermediate 6-1 to obtain Compound 226 (8.0 mmol, Y=80%).

### 2. Manufacture and Evaluation of Light Emitting Elements

A light emitting element of an embodiment including the amine compound of an embodiment in a hole transport layer was manufactured by a method below. Light emitting elements of Example 1 to Example 24 were manufactured using the amine compounds of Compounds 1, 6, 21, 26, 41, 46, 61, 66, 81, 86, 101, 106, 121, 126, 141, 146, 161, 166, 181, 186, 201, 206, 221 and 226, which are the above-explained Example Compounds. Comparative Example 1 to Comparative Example 6 correspond to light emitting elements manufactured using Comparative Compounds C1 to C6 as hole transport materials.

### Example Compounds

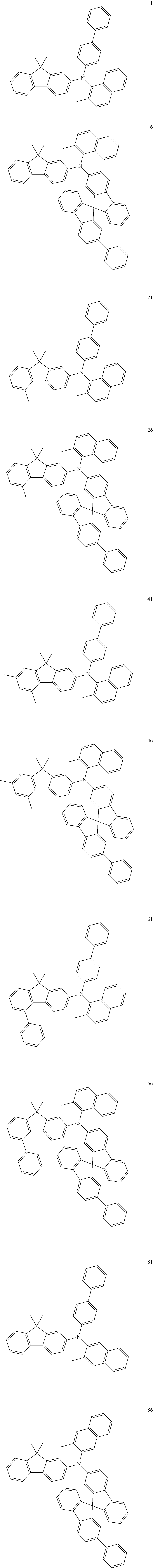

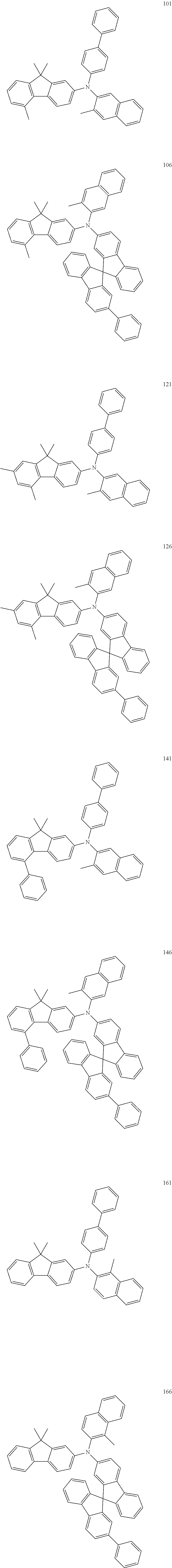

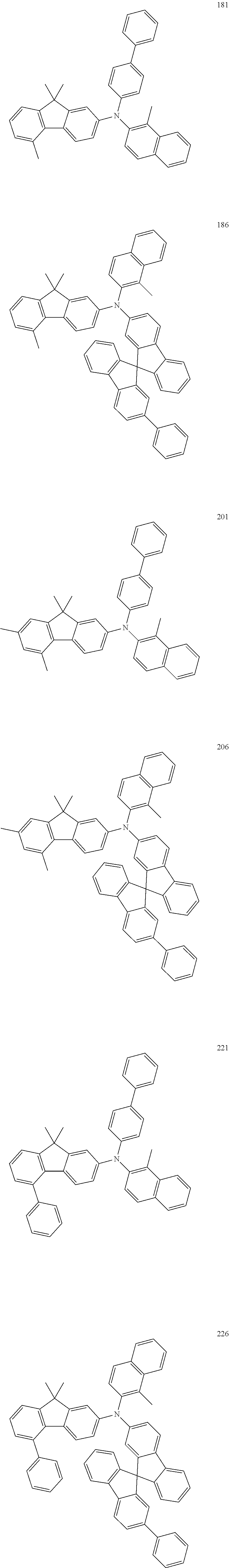

### Comparative Compounds

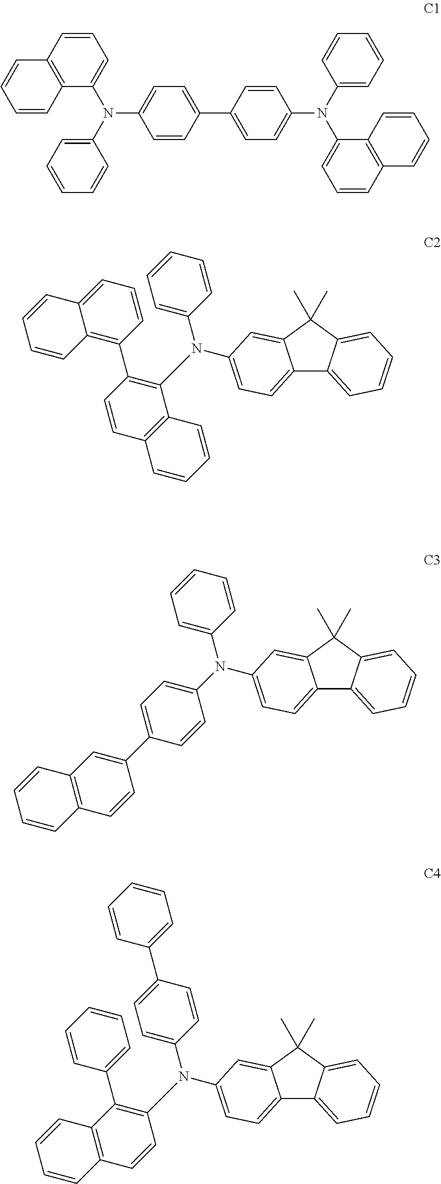 [SMILES: c1ccc(N(c2ccc(-c3ccc(N(c4ccccc4)c4cccc5ccccc45)cc3)cc2)c2cccc3ccccc23)cc1 ; CC1(C)c2ccccc2-c2ccc(N(c3ccccc3)c3c(-c4cccc5ccccc45)ccc4ccccc34)cc21 ; CC1(C)c2ccccc2-c2ccc(N(c3ccccc3)c3ccc(-c4ccc5ccccc5c4)cc3)cc21 ; CC1(C)c2ccccc2-c2ccc(N(c3ccc(-c4ccccc4)cc3)c3ccc4ccccc4c3-c3ccccc3)cc21]

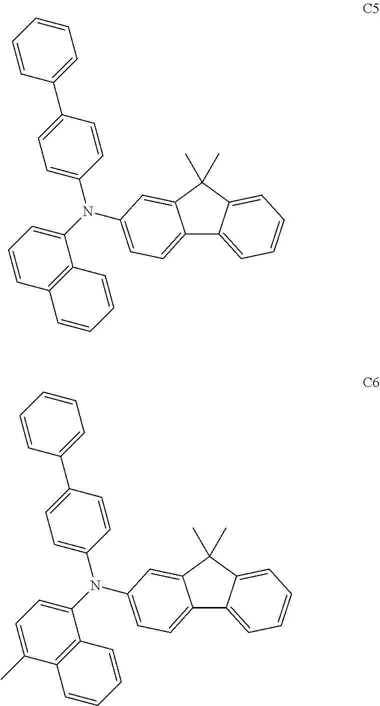 [SMILES: CC1(C)c2ccccc2-c2ccc(N(c3ccc(-c4ccccc4)cc3)c3cccc4ccccc34)cc21 ; Cc1ccc(N(c2ccc(-c3ccccc3)cc2)c2ccc3c(c2)C(C)(C)c2ccccc2-3)c2ccccc12]

### Manufacture of Light Emitting Elements

An ITO glass substrate with about 15 Ω/cm² (about 1200 Å) of Corning Co. was cut into a size of 50 mm×50 mm×0.7 mm, washed with isopropyl alcohol and ultrapure water, cleansed using ultrasonic waves for about 5 minutes, exposed to UV for about 30 minutes and treated with ozone. Then, 4,4′,4″-tris{N,-(2-naphthyl)-N-phenylamino}-triphenylamine (2-TNATA) was vacuum deposited to a thickness of about 600 Å to form a hole injection layer. After that, the Example Compound or Comparative Compound was vacuum deposited to a thickness of about 300 Å to form a hole transport layer.

On the hole transport layer, a blue fluorescence host of 9,10-di(naphthalen-2-yl)anthracene (ADN) and 4,4′-bis[2-(4-(N,N-diphenylamino)phenyl)vinyl]biphenyl (DPAVBi) were co-deposited in a ratio of about 98:2 to form an emission layer with a thickness of about 300 Å.

On the emission layer, an electron transport layer was formed to a thickness of about 300 Å using tris(8-hydroxyquinolino)aluminum (Alq₃), and then, an electron injection layer was formed to a thickness of about 10 Å by depositing LiF. On the electron injection layer, a second electrode was formed to a thickness of about 3000 Å using aluminum (Al).

In addition, the compounds of the functional layers used for the manufacture of the light emitting elements are as follows.

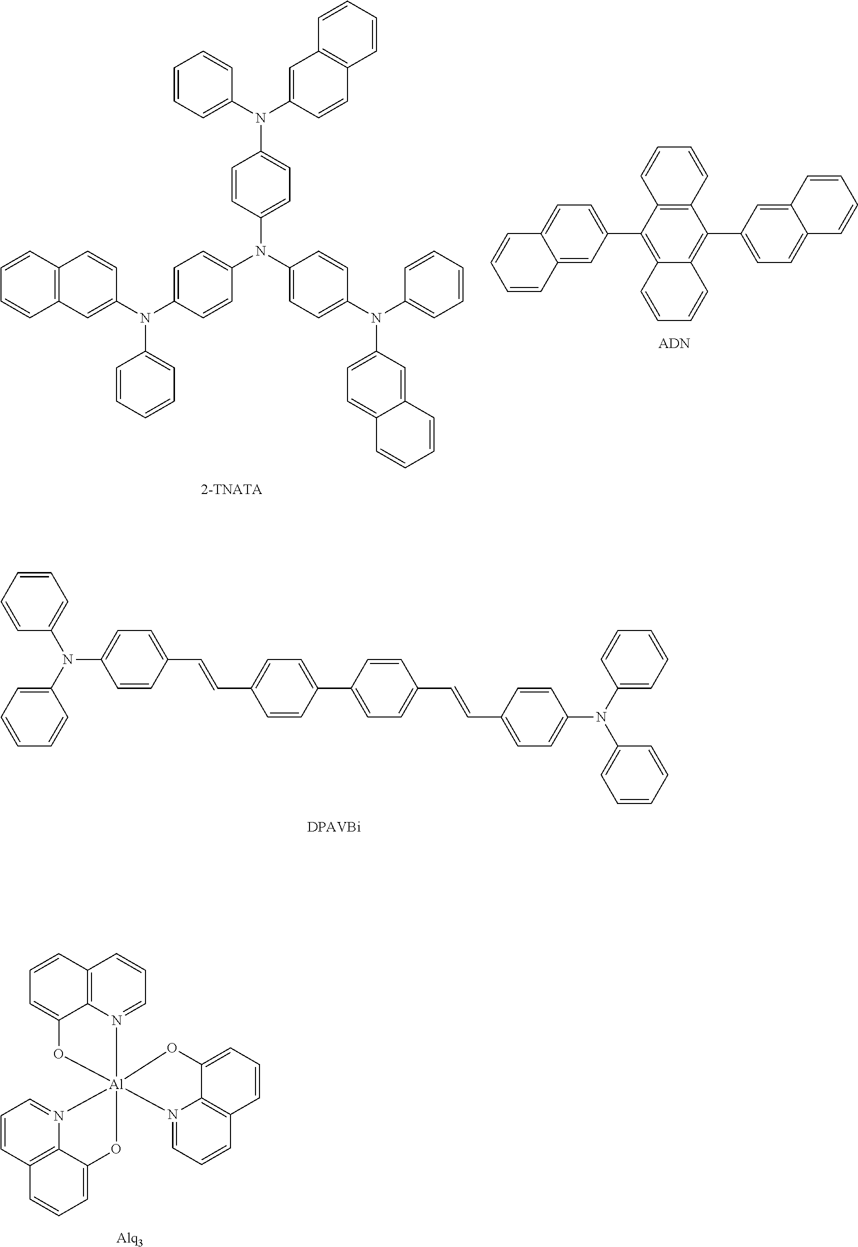

### (2) Evaluation of Light Emitting Elements

Table 1 shows evaluation results of the light emitting elements of Examples 1 to 10, and Comparative Examples 1 to 5. In Table 1, the luminance, emission efficiency and half life of the light emitting elements manufactured are shown.

In the evaluation results of the properties of the Examples and Comparative Examples, shown in Table 1, the voltage and current density were measured using V7000 OLED IVL Test System (Polaronix). The emission efficiency shows efficiency values at a current density of 50 mA/cm². The half life shows luminance half life measured at a current density of 100 mA/cm².

TABLE 1

| | | | | | | | Half life |
|---|---|---|---|---|---|---|---|
| | | Driving | Current | | Efficien- | | (hr |
| | Hole transport | voltage | density | Luminance | cy | Emission | @100mA/ |
| | layer material | (V) | (mA/cm²) | (cd/m²) | (cd/A) | color | cm²) |
| Example 1 | Compound 1 | 4.78 | 50 | 3240 | 6.48 | Blue | 520 |
| Example 2 | Compound 6 | 4.81 | 50 | 3260 | 6.52 | Blue | 550 |
| Example 3 | Compound 21 | 4.65 | 50 | 3210 | 6.42 | Blue | 580 |
| Example 4 | Compound 26 | 4.75 | 50 | 3200 | 6.40 | Blue | 540 |
| Example 5 | Compound 41 | 4.52 | 50 | 3400 | 6.80 | Blue | 590 |
| Example 6 | Compound 46 | 4.52 | 50 | 3500 | 7.00 | Blue | 570 |
| Example 7 | Compound 61 | 4.50 | 50 | 3400 | 6.80 | Blue | 560 |
| Example 8 | Compound 66 | 4.61 | 50 | 3350 | 6.70 | Blue | 560 |
| Example 9 | Compound 81 | 4.81 | 50 | 3300 | 6.60 | Blue | 550 |
| Example 10 | Compound 86 | 4.49 | 50 | 3280 | 6.56 | Blue | 550 |
| Example 11 | Compound 101 | 4.78 | 50 | 3240 | 6.48 | Blue | 520 |
| Example 12 | Compound 106 | 4.71 | 50 | 3260 | 6.52 | Blue | 550 |
| Example 13 | Compound 121 | 4.51 | 50 | 3400 | 6.80 | Blue | 580 |
| Example 14 | Compound 126 | 4.45 | 50 | 3380 | 6.76 | Blue | 570 |
| Example 15 | Compound 141 | 4.52 | 50 | 3300 | 6.60 | Blue | 590 |
| Example 16 | Compound 146 | 4.52 | 50 | 3220 | 6.44 | Blue | 570 |
| Example 17 | Compound 161 | 4.65 | 50 | 3200 | 6.40 | Blue | 560 |
| Example 18 | Compound 166 | 4.55 | 50 | 3290 | 6.58 | Blue | 590 |
| Example 19 | Compound 181 | 4.71 | 50 | 3300 | 6.60 | Blue | 550 |
| Example 20 | Compound 186 | 4.69 | 50 | 3280 | 6.56 | Blue | 520 |
| Example 21 | Compound 201 | 4.55 | 50 | 3340 | 6.68 | Blue | 530 |
| Example 22 | Compound 206 | 4.53 | 50 | 3590 | 7.18 | Blue | 560 |
| Example 23 | Compound 221 | 4.51 | 50 | 3300 | 6.60 | Blue | 580 |
| Example 24 | Compound 226 | 4.59 | 50 | 3280 | 6.56 | Blue | 550 |
| Comparative | Comparative | 7.01 | 50 | 2645 | 5.29 | Blue | 258 |
| Example 1 | Compound C1 | | | | | | |
| Comparative | Comparative | 5.18 | 50 | 2050 | 4.10 | Blue | 320 |
| Example 2 | Compound C2 | | | | | | |
| Comparative | Comparative | 5.20 | 50 | 2150 | 4.30 | Blue | 380 |
| Example 3 | Compound C3 | | | | | | |
| Comparative | Comparative | 5.00 | 50 | 3000 | 6.00 | Blue | 450 |
| Example 4 | Compound C4 | | | | | | |
| Comparative | Comparative | 4.99 | 50 | 2950 | 5.90 | Blue | 400 |
| Example 5 | Compound C5 | | | | | | |
| Comparative | Comparative | 5.30 | 50 | 3300 | 6.60 | Blue | 420 |
| Example 6 | Compound C6 | | | | | | |

Referring to the results of Table 1, it can be seen that the light emitting elements of the Examples using the amine compounds according to embodiments of the present disclosure as the hole transport layer materials, showed relatively low driving voltage values, high luminance and high emission efficiency while emitting the same blue color light when compared to the Comparative Examples. The Example Compounds are tertiary amine compounds having a structure in which a first substituent, a second substituent and a third substituent are connected with an amine group. The first substituent is an alkyl-substituted naphthalene group. If the carbon atom at position 1 of the naphthalene moiety of the first substituent is connected with the amine group, the carbon atom at position number 2 may be connected with the alkyl group, and if the carbon atom at position 2 of the naphthalene moiety of the first substituent is connected with the amine group, at least one carbon atom at position number 1 or 3 may be connected with the alkyl group. The Example Compounds further include the second substituent including a fluorenyl moiety and the third substituent such as an aryl group. The amine compound of an embodiment, having such a structure has low refractive index properties and improved hole transport capacity, and if applied to a light emitting element, the improvement of the emission efficiency and the increase of the lifetime of the light emitting element may be accomplished. In some embodiments, the light emitting element of an embodiment includes the amine compound of an embodiment as the hole transport layer material of the light emitting element, and the efficiency and lifetime of the light emitting element may be improved.

On the contrary, Comparative Compound C1 included in Comparative Example 1, does not include the first substituent and the second substituent. Accordingly, if Comparative Compound C1 is applied to a light emitting element, it is thought that the emission efficiency and lifetime characteristics were deteriorated in contrast to the Example Compounds.

Comparative Compounds C2 to C5, included in Comparative Examples 2 to 5, do not include the first substituent. Comparative Compounds C2 to C5 include a naphthalene moiety in a substituent but do not include an alkyl group bonded thereto. Accordingly, it is thought that if Comparative Compounds C2 to C5 were applied to light emitting elements, emission efficiency and lifetime characteristics were deteriorated when compared to the Example Compounds.

Comparative Compound C6 included in Comparative Example 6 does not include the first substituent. Comparative Compound C6 includes an alkyl-bonded naphthyl group in a substituent, but the alkyl group is not bonded at the ortho position to the naphthyl group. Accordingly, it is thought that if Comparative Compound C6 was applied to a light emitting element, emission efficiency and lifetime characteristics were deteriorated when compared to the Example Compounds.

If the Comparative Compounds are applied to light emitting elements, it can be seen that luminance and emission efficiency were reduced, and half life was reduced when compared to the Example Compounds. That is, referring to Table 1, it can be seen that the light emitting element using the amine compound of an embodiment of the present disclosure showed improved element properties in view of emission efficiency or element lifetime when compared to the Comparative Examples.

The light emitting element of an embodiment includes the amine compound of an embodiment and may show high efficiency and long-life characteristics.

If the amine compound of an embodiment is applied to a light emitting element, high efficiency and long-life characteristics may be shown.

Although example embodiments of the present disclosure have been described, it is understood that the present disclosure should not be limited to these embodiments, but various changes and modifications can be made by one ordinary skilled in the art within the spirit and scope of the present disclosure as defined by the appended claims, and equivalents thereof.

What is claimed is:

1. A light emitting element, comprising:
a first electrode;
a second electrode on the first electrode; and
at least one functional layer between the first electrode and the second electrode, and comprising an amine compound represented by the following Formula 1: 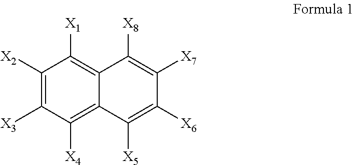
in Formula 1,
X₁ is a hydrogen atom, a deuterium atom, a halogen atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or represented by the following Formula 2,
X₂ is a substituted or unsubstituted alkyl group of 1 to 4 carbon atoms, or represented by the following Formula 2,
X₃ to X₈ are each independently a hydrogen atom, a deuterium atom, a halogen atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms,
any one selected from among X₁ and X₂ is represented by the following Formula 2,
if X₁ is represented by the following Formula 2, X₂ is a substituted or unsubstituted alkyl group of 1 to 4 carbon atoms, and
if X₂ is represented by the following Formula 2, any one selected from among X₁ and X₃ is a substituted or unsubstituted alkyl group of 1 to 4 carbon atoms: 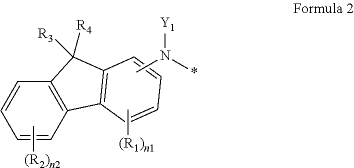
in Formula 2,
R₁ to R₄ are each independently a hydrogen atom, a deuterium atom, a halogen atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms,
Y₁ is a substituted or unsubstituted alkyl group of 1 to 20 carbon atoms, a substituted or unsubstituted alkenyl group of 2 to 20 carbon atoms, a substituted or unsubstituted alkynyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms,
n1 is an integer of 0 to 3,
n2 is an integer of 0 to 4, and
“-*” is a position connected with Formula 1.

2. The light emitting element of claim 1, wherein the at least one functional layer comprises an emission layer, a hole transport region between the first electrode and the emission layer, and an electron transport region between the emission layer and the second electrode, and
the hole transport region comprises the amine compound represented by Formula 1.

3. The light emitting element of claim 2, wherein the hole transport region comprises a hole injection layer on the first electrode, and a hole transport layer on the hole injection layer, and
the hole transport layer comprises the amine compound represented by Formula 1.

4. The light emitting element of claim 1, wherein the amine compound represented by Formula 1 is a monoamine compound.

5. The light emitting element of claim 1, wherein, in Formula 2, Y₁ is a substituted or unsubstituted phenyl group, a substituted or unsubstituted naphthyl group, a substituted or unsubstituted fluorenyl group, or a substituted or unsubstituted carbazole group.

6. The light emitting element of claim 1, wherein the amine compound represented by Formula 1 is represented by any one selected from among the following Formula 1-1 to Formula 1-3: 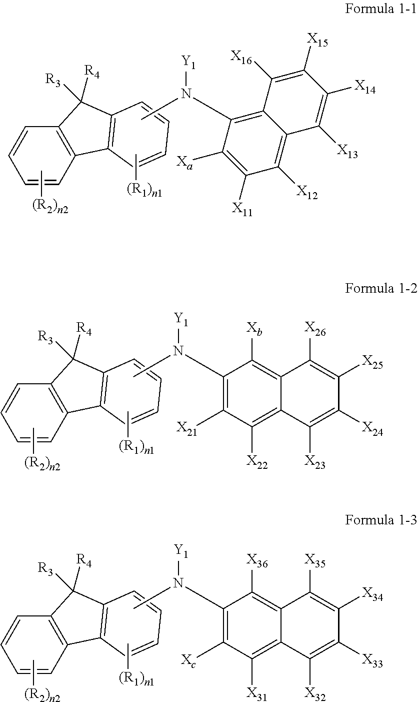
in Formula 1-1 to Formula 1-3,
Xₐ, X_{b} and X_{c} are each independently a substituted or unsubstituted alkyl group of 1 to 4 carbon atoms,
X₁₁ to X₃₆ are each independently a hydrogen atom, a deuterium atom, a halogen atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and
R₁ to R₄, n1, n2 and Y₁ are the same as defined with respect to Formula 2.

7. The light emitting element of claim 6, wherein, in Formula 1-1 to Formula 1-3, Xₐ to X_{c} are each independently a substituted or unsubstituted methyl group.

8. The light emitting element of claim 6, wherein, in Formula 1-1 to Formula 1-3, X₁₁ to X₃₆ are each independently a hydrogen atom or a deuterium atom.

9. The light emitting element of claim 1, wherein the substituent represented by Formula 1 is represented by the following Formula 2-1: 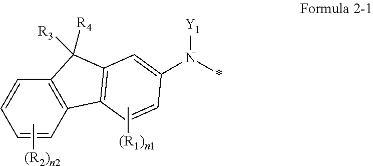
in Formula 2-1,
R₁ to R₄, n1, n2 and Y₁ are the same as defined with respect to Formula 2.

10. The light emitting element of claim 1, wherein the substituent represented by Formula 2 is represented by the following Formula 2-2: 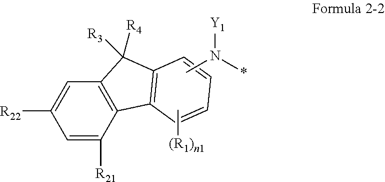
in Formula 2-2,
R₂₁ and R₂₂ are each independently a hydrogen atom, a substituted or unsubstituted alkyl group of 1 to 20 carbon atoms, or a substituted or unsubstituted aryl group of 6 to 30 ring-forming carbon atoms, and
R₁, R₃, R₄, n1 and Y₁ are the same as defined with respect to Formula 2.

11. The light emitting element of claim 1, wherein the substituent represented by Formula 2 is represented by the following Formula 2-3: 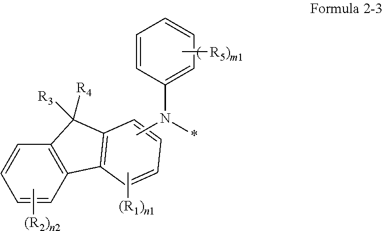
in Formula 2-3,
R₅ is a hydrogen atom, a deuterium atom, a halogen atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring,
m1 is an integer of 0 to 5, and
R₁ to R₄, n1 and n2 are the same as defined with respect to Formula 2.

12. The light emitting element of claim 1, wherein the amine compound represented by Formula 1 is represented by any one selected from among compounds in the following Compound Group 1:

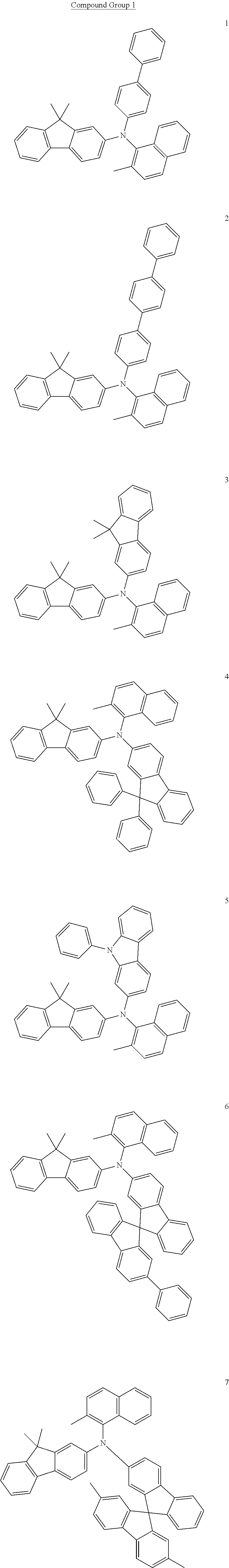

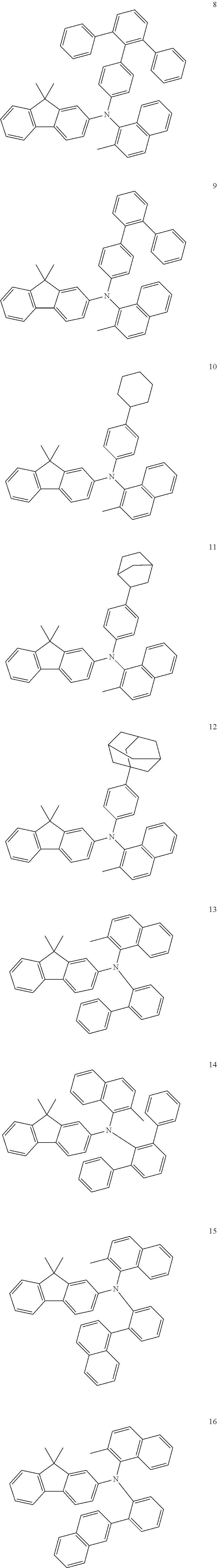

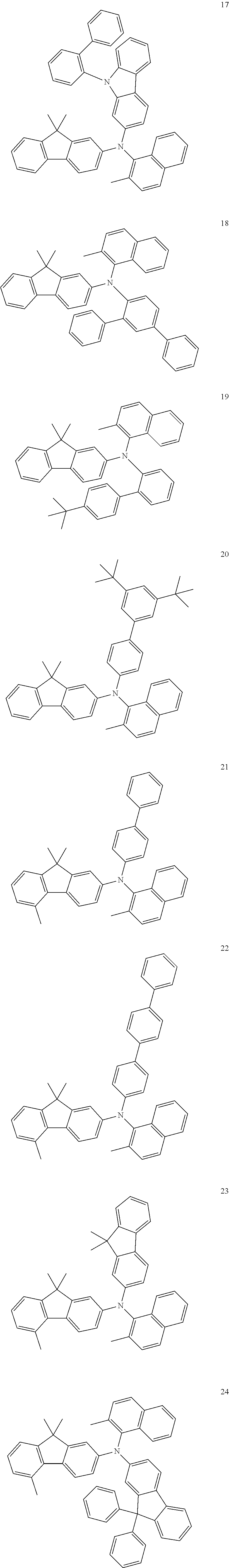

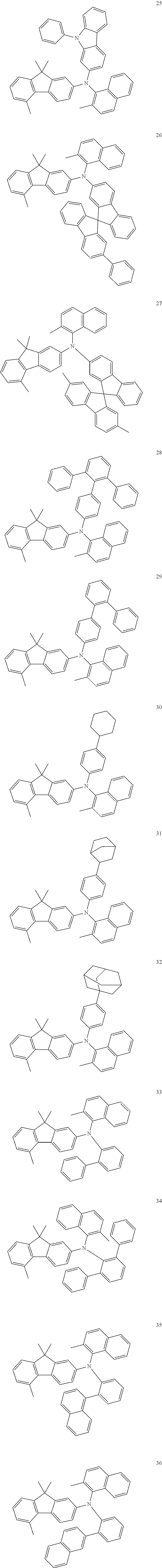

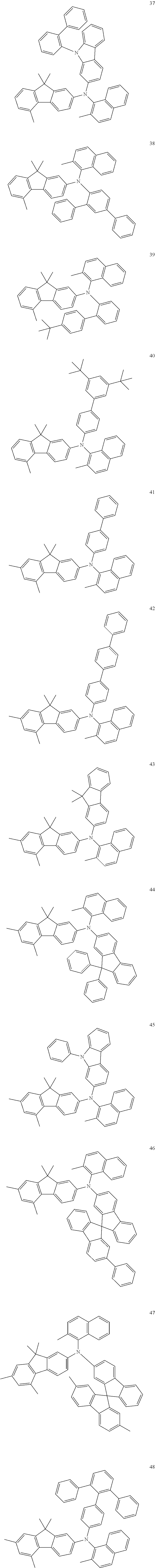

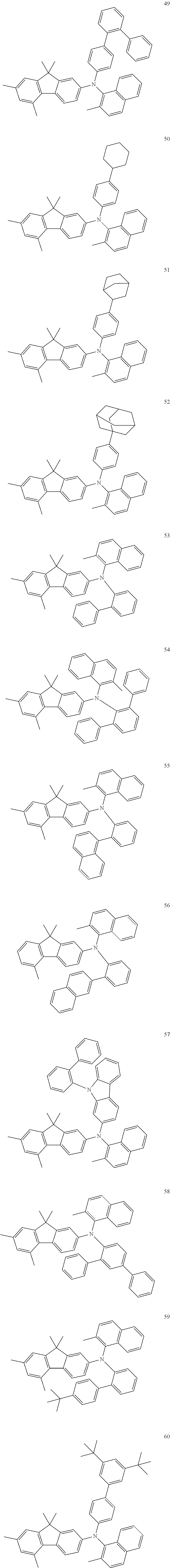

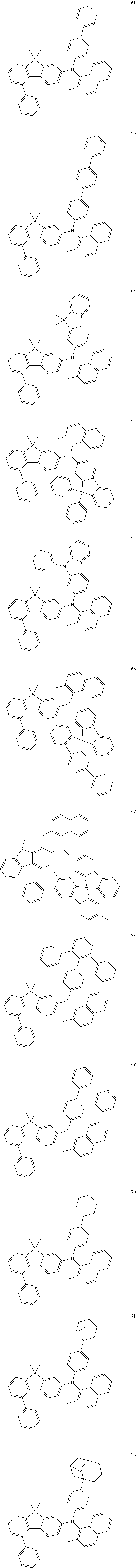

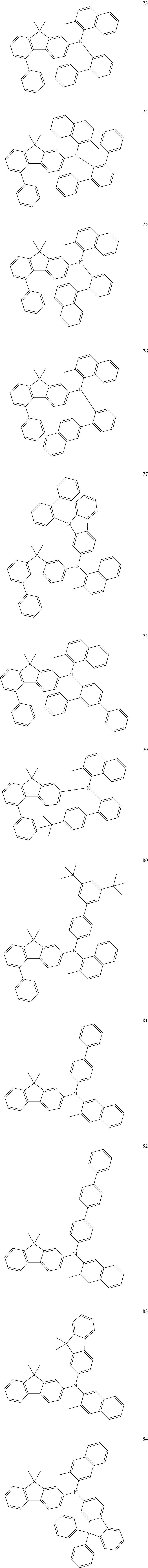

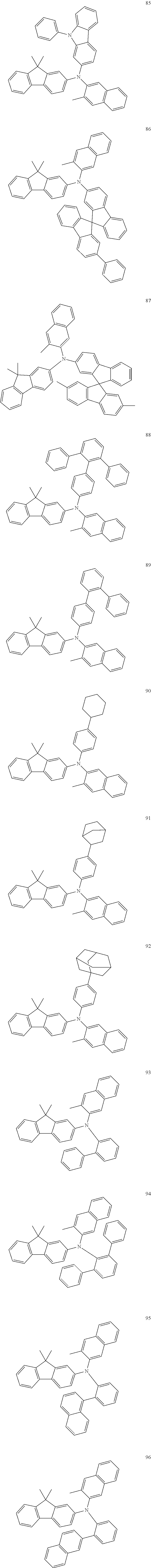

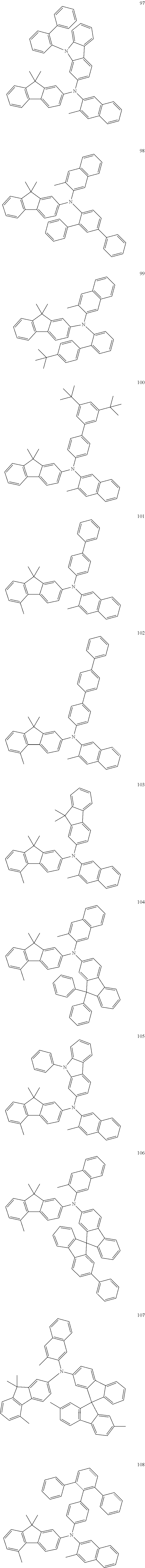

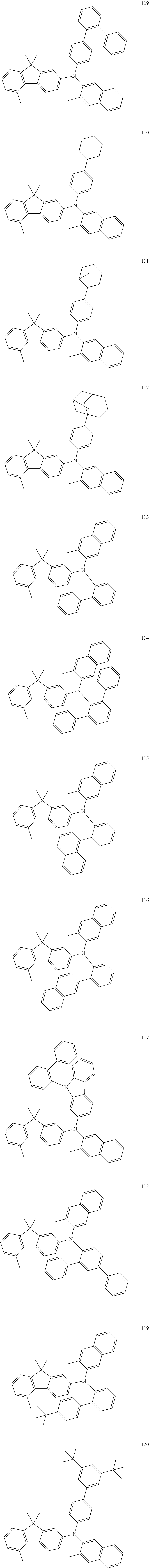

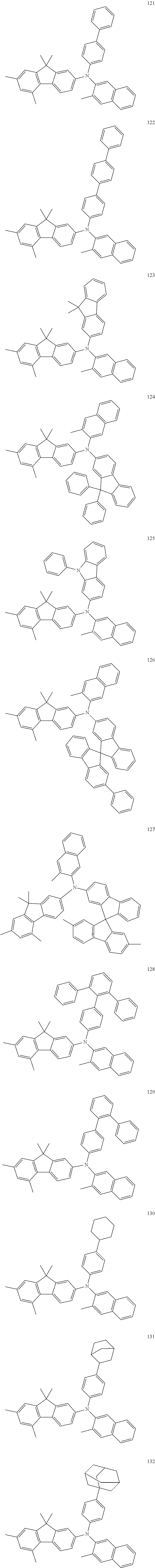

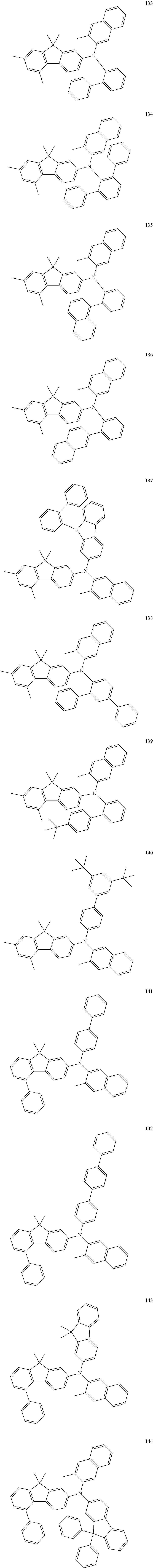

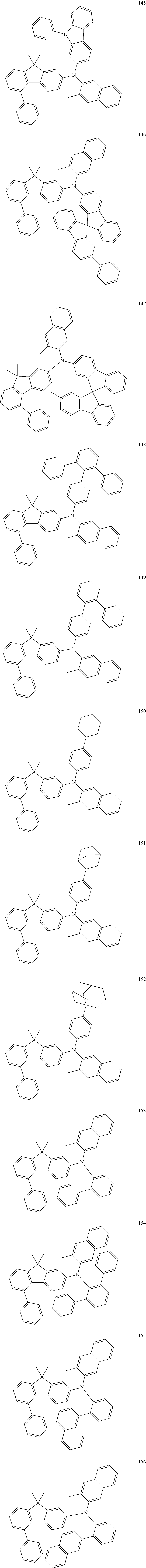

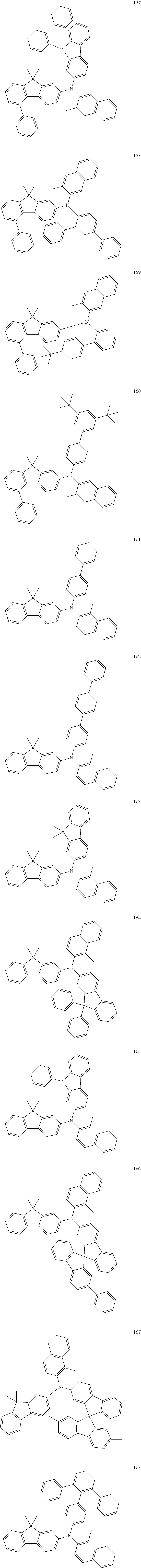

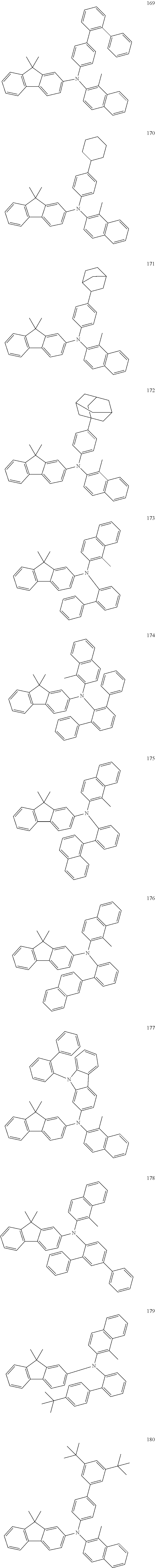

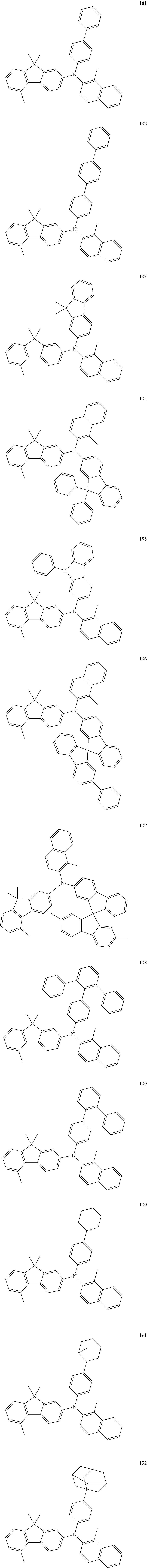

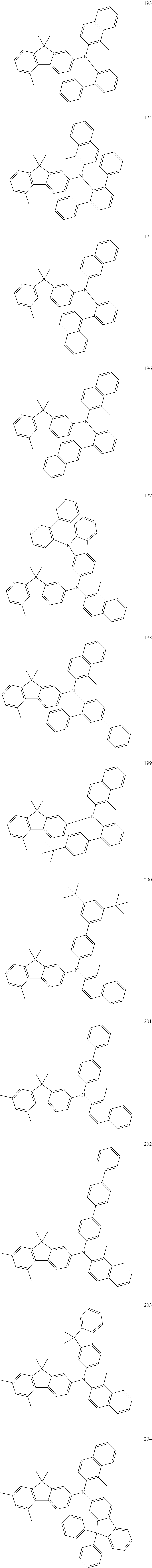

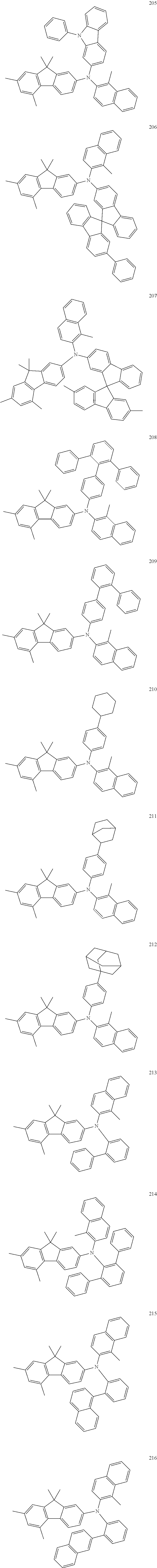

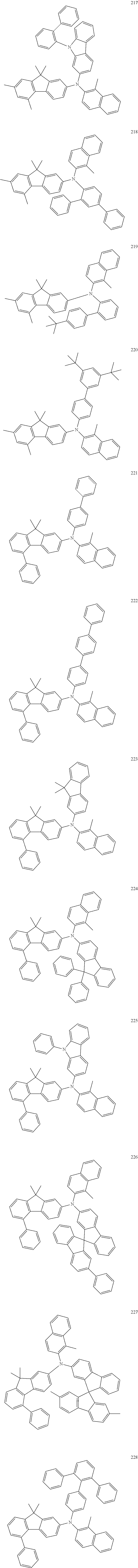

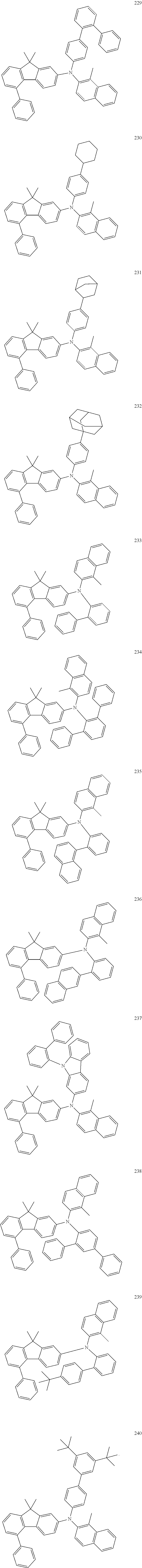

13. An amine compound represented by the following Formula 1: 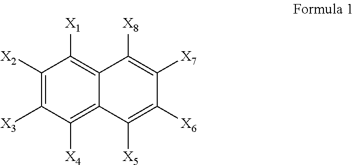
in Formula 1,
X₁ is a hydrogen atom, a deuterium atom, a halogen atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or represented by the following Formula 2,
X₂ is a substituted or unsubstituted alkyl group of 1 to 4 carbon atoms, or represented by the following Formula 2,
X₃ to X₈ are each independently a hydrogen atom, a deuterium atom, a halogen atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms,
any one selected from among X₁ and X₂ is represented by the following Formula 2,
if X₁ is represented by the following Formula 2, X₂ is a substituted or unsubstituted alkyl group of 1 to 4 carbon atoms, and
if X₂ is represented by the following Formula 2, any one selected from among X₁ and X₃ is a substituted or unsubstituted alkyl group of 1 to 4 carbon atoms: 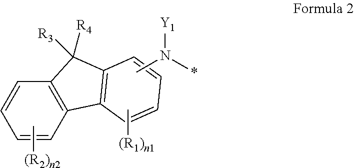
in Formula 2,
R₁ to R₄ are each independently a hydrogen atom, a deuterium atom, a halogen atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms,
Y₁ is a substituted or unsubstituted alkyl group of 1 to 20 carbon atoms, a substituted or unsubstituted alkenyl group of 2 to 20 carbon atoms, a substituted or unsubstituted alkynyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms,
n1 is an integer of 0 to 3,
n2 is an integer of 0 to 4, and
“-*” is a position connected with Formula 1.

14. The amine compound of claim 13, wherein the amine compound represented by Formula 1 is represented by any one selected from among the following Formula 1-1 to Formula 1-3: 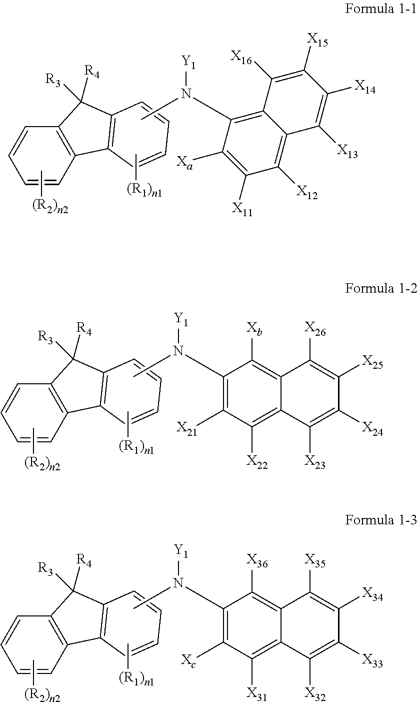
in Formula 1-1 to Formula 1-3,
Xₐ, X_{b} and X_{c} are each independently a substituted or unsubstituted alkyl group of 1 to 4 carbon atoms,
X₁₁ to X₃₆ are each independently a hydrogen atom, a deuterium atom, a halogen atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and
R₁ to R₄, n1, n2 and Y₁ are the same as defined with respect to Formula 2.

15. The amine compound of claim 14, wherein, in Formula 1-1 to Formula 1-3, Xₐ to X_{c} are each independently a substituted or unsubstituted methyl group.

16. The amine compound of claim 14, wherein, in Formula 1-1 to Formula 1-3, X₁₁ to X₃₆ are each independently a hydrogen atom or a deuterium atom.

17. The amine compound of claim 13, wherein the substituent represented by Formula 2 is represented by the following Formula 2-1: 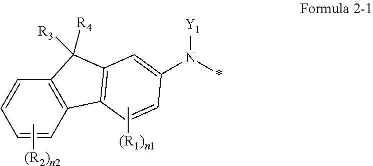
in Formula 2-1,
R₁ to R₄, n1, n2 and Y₁ are the same as defined with respect to Formula 2.

18. The amine compound of claim 13, wherein the substituent represented by Formula 2 is represented by the following Formula 2-2: 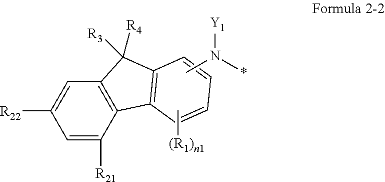
in Formula 2-2,
R₂₁ and R₂₂ are each independently a hydrogen atom, a substituted or unsubstituted alkyl group of 1 to 20 carbon atoms, or a substituted or unsubstituted aryl group of 6 to 30 ring-forming carbon atoms, and
R₁, R₃, R₄, n1 and Y₁ are the same as defined with respect to Formula 2.

19. The amine compound of claim 13, wherein the substituent represented by Formula 2 is represented by the following Formula 2-3: 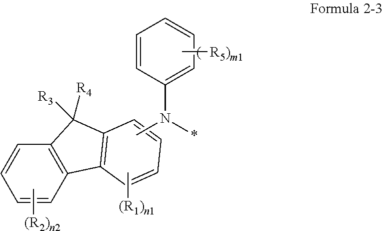
in Formula 2-3,
R₅ is a hydrogen atom, a deuterium atom, a halogen atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring,
m1 is an integer of 0 to 5, and
R₁ to R₄, n1 and n2 are the same as defined with respect to Formula 2.

20. The amine compound of claim 13, wherein the amine compound represented by Formula 1 is represented by any one selected from among compounds in the following Compound Group 1: 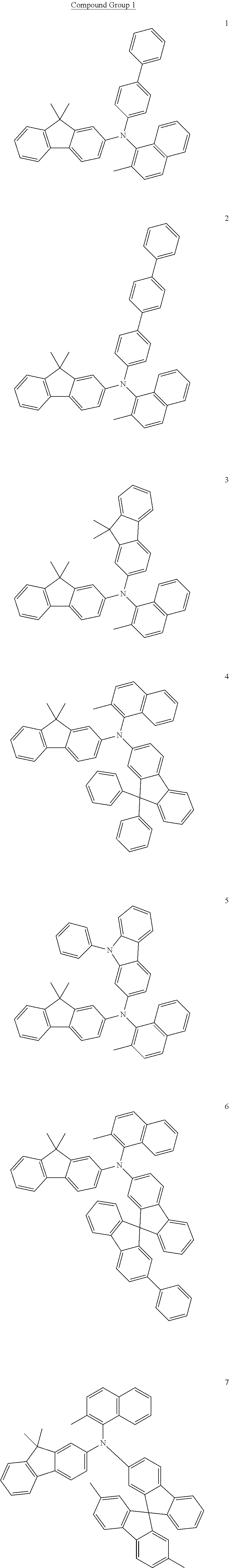 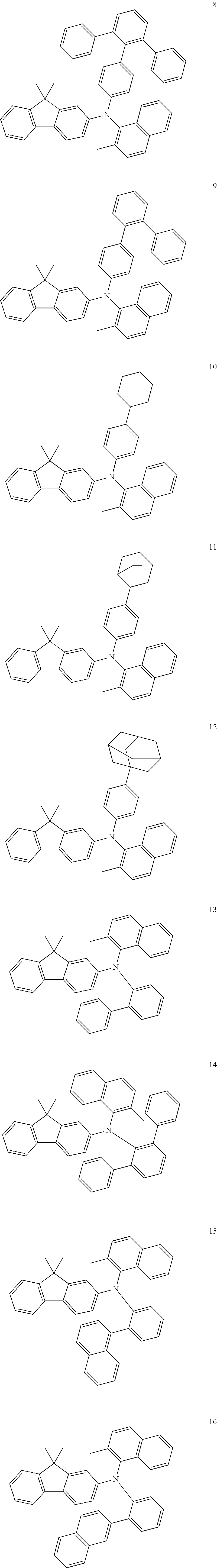 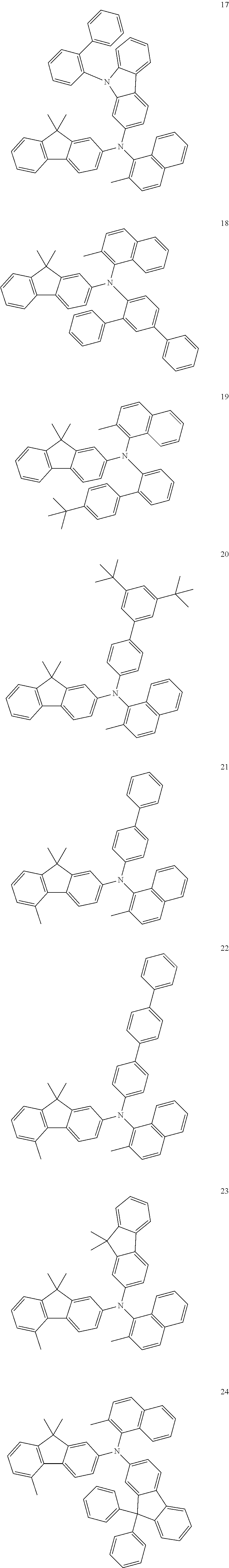 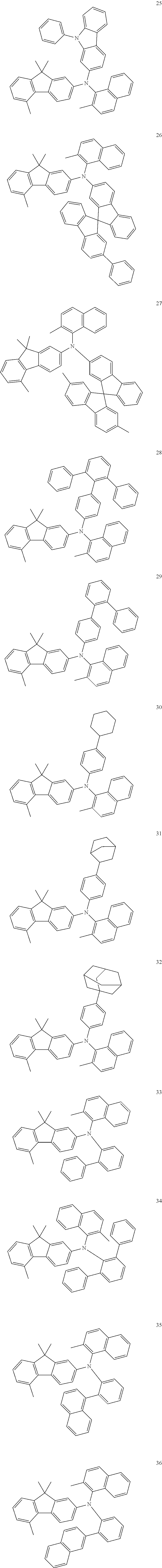 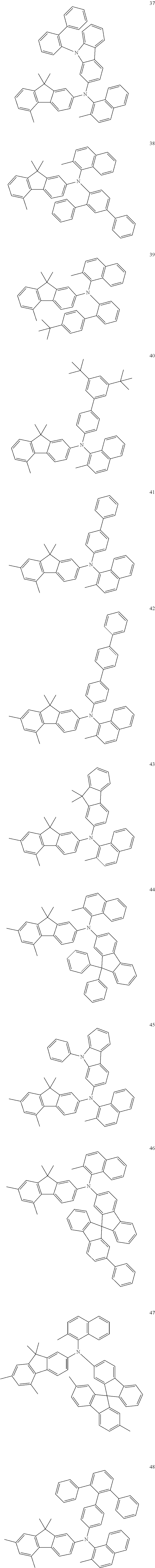 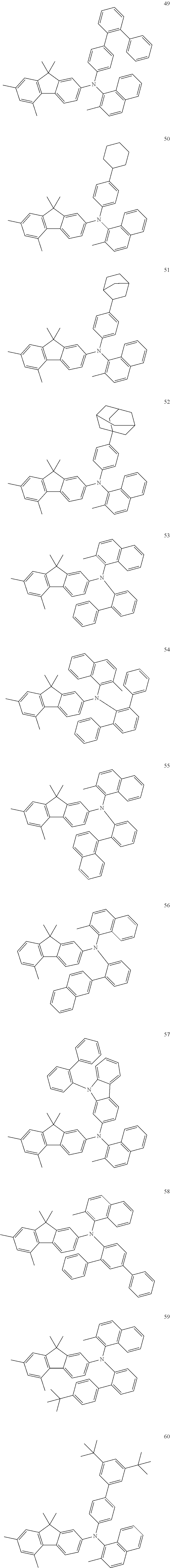 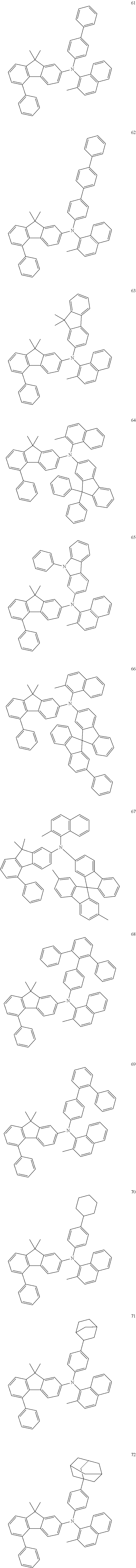 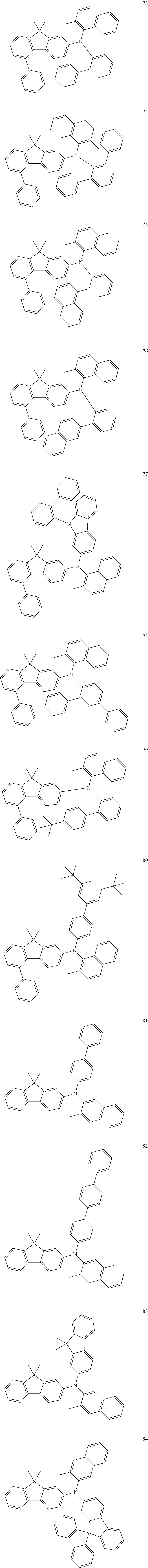 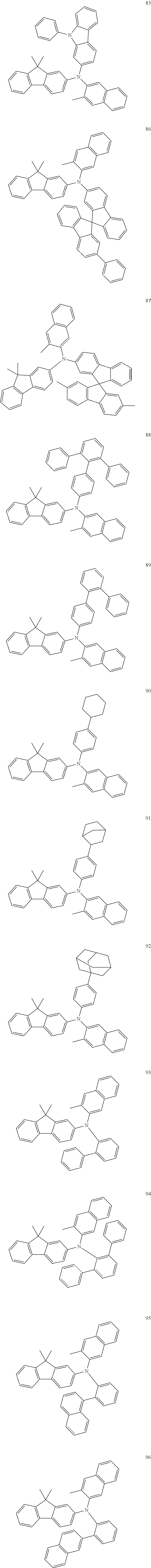 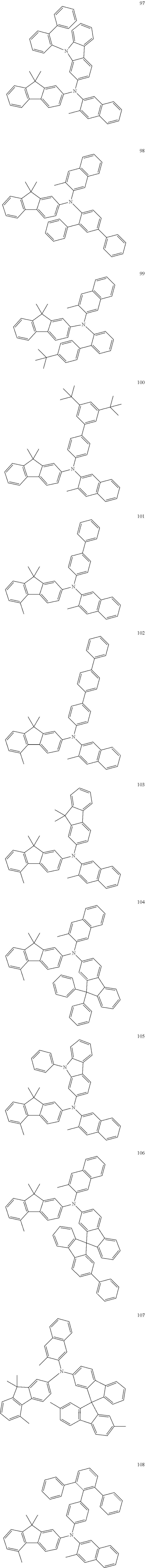 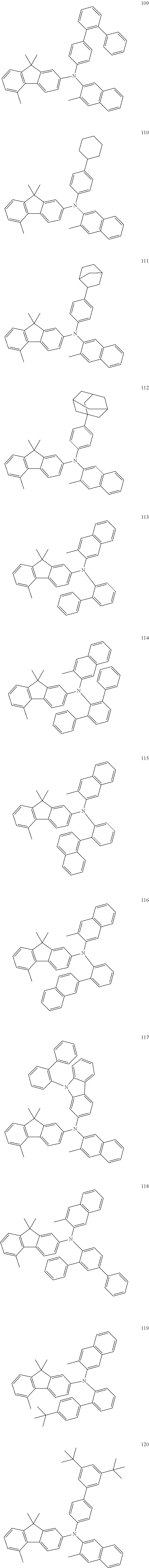 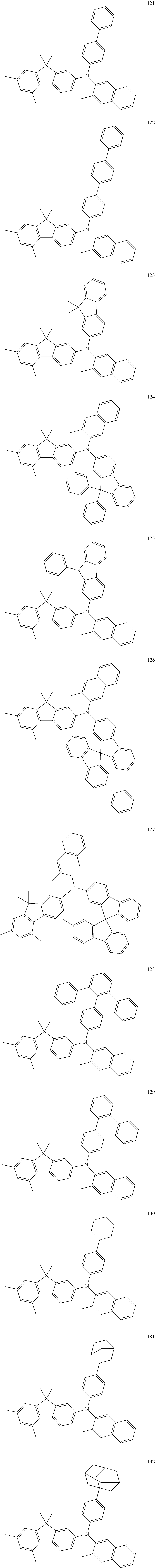 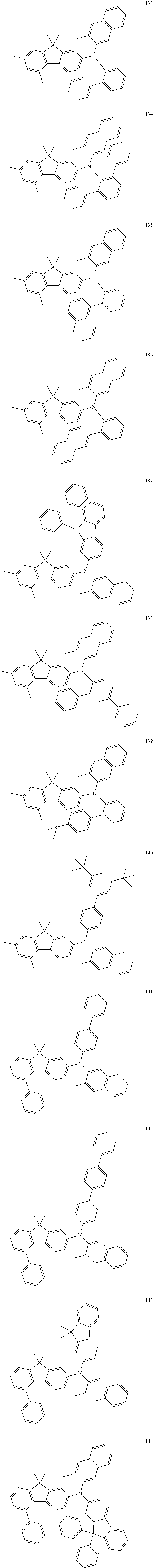 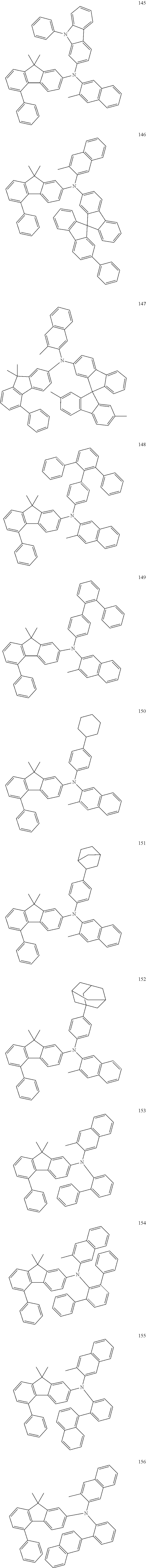 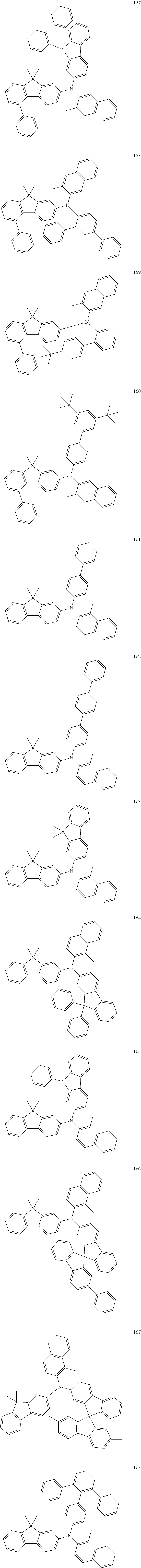 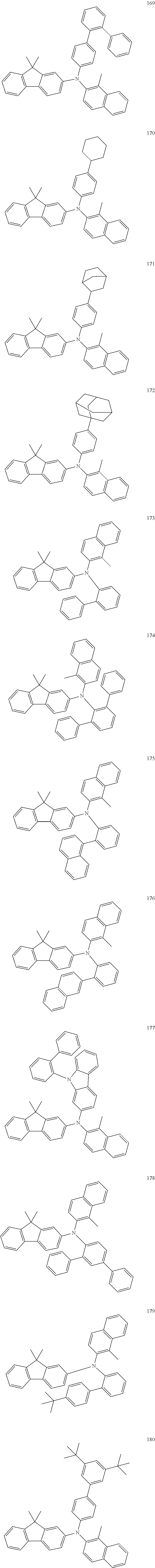 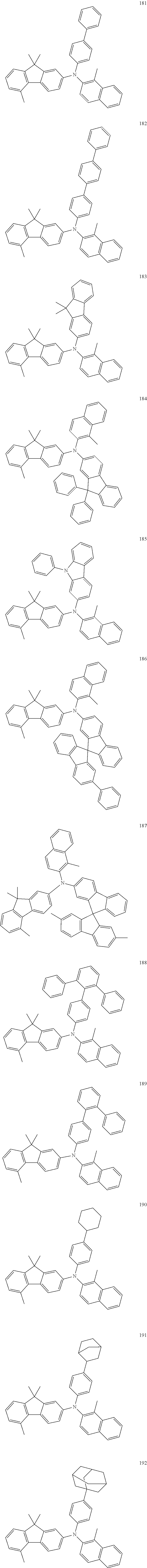 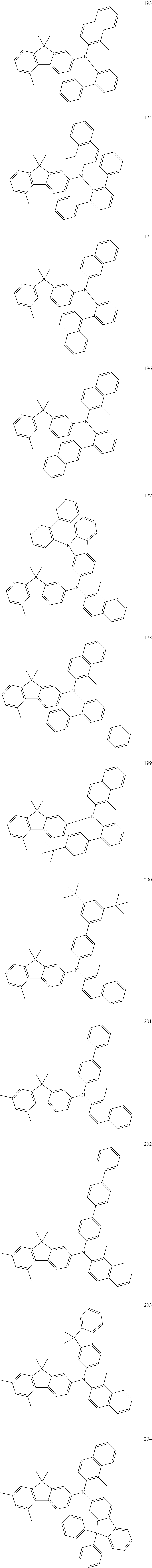 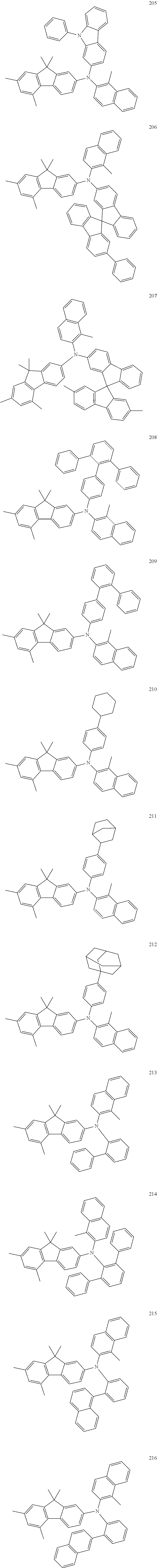 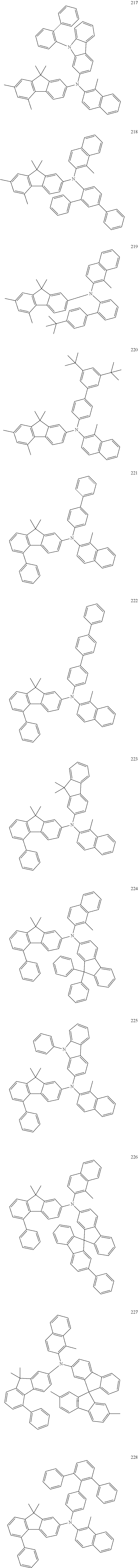 [chemical structure]
